SEQUENCE LISTING (213 sequences)

SEQ ID NO: 1 - RNA, 33 nt, Homo sapiens
auaaacuugugguguggagcugguggcguaggc

SEQ ID NO: 2 - DNA, 20 bp, Artificial Sequence
  rSBL primer for human KRAS G12 codon point mutation
agctccacaccacaagttta

SEQ ID NO: 3 - DNA, 32 bp, Artificial Sequence
  expected wild-type rSBL product of human KRAS G12 codon
gcctacgccaccagctccacaccacaagttta

SEQ ID NO: 4 - DNA, 32 bp, Artificial Sequence
  non-wild type complementary sequence for human KRAS G12 codon
gcctacgccbddagctccacaccacaagttta

SEQ ID NO: 5 - DNA, 12 bp, Artificial Sequence
  rSBL probe for detection of human KRAS G12 codon point mutation
gcctacgccbcc

SEQ ID NO: 6 - DNA, 12 bp, Artificial Sequence
  rSBL probe for detection of human KRAS G12 codon point mutation
gcctacgccadc

SEQ ID NO: 7 - DNA, 12 bp, Artificial Sequence
  rSBL probe for detection of human KRAS G12 codon point mutation
gcctacgccacd

SEQ ID NO: 8 - DNA, 12 bp, Artificial Sequence
  rSBL probe for wild-type sequence of human KRAS G12 codon point mutation
gcctacgccacc

SEQ ID NO: 9 - DNA, 40 bp, Artificial Sequence
  rSBL primer for detecting G12A Kras mutations
  misc_feature (1)..(20): PCR adapter sequence, in situ PCR adapter sequence, or in situ signal amplification handle
  misc_feature (38)..(40): phosphorothioate bond
agctccaactaccacaagttcaggatacacactacccgtg

SEQ ID NO: 10 - DNA, 32 bp, Artificial Sequence
  rSBL probe for detecting G12A Kras mutation
  misc_feature (1)..(20): PCR adapter sequence, in situ PCR adapter sequence, or in situ signal amplification handle
  misc_feature (30)..(32): n is a, c, g, or t
gggtcatatcggtcactgttgcctacgccnnn

SEQ ID NO: 11 - DNA, 32 bp, Artificial Sequence
  rSBL probe for detecting G12A Kras mutation
gggtcatatcggtcactgttgcctacgccbcc

SEQ ID NO: 12 - DNA, 32 bp, Artificial Sequence
  rSBL probe for detecting G12A Kras mutation
  misc_feature (1)..(20): PCR adapter sequence, in situ PCR adapter sequence, or in situ signal amplification handle
gggtcatatcggtcactgttgcctacgccadc

SEQ ID NO: 13 - DNA, 32 bp, Artificial Sequence
  rSBL probe for detecting G12A Kras mutation
  misc_feature: PCR adapter sequence, in situ PCR adapter sequence, or in situ signal amplification handle
gggtcatatcggtcactgttgcctacgccacd

SEQ ID NO: 14 - DNA, 12 bp, Artificial Sequence
  rSBL probe for detecting G12A Kras mutation
gcctacgccacc

SEQ ID NO: 15 - DNA, 40 bp, Artificial Sequence
  SBL primer for Q61K mutation
acctgctgtgtcgagaatatcaggatacacactacccgtg

SEQ ID NO: 16 - DNA, 32 bp, Artificial Sequence
  SBL probe for Q61K mutation
  misc_feature (30)..(32): n is a, c, g, or t
gggtcatatcggtcactgttgtactcctcnnn

SEQ ID NO: 17 - DNA, 32 bp, Artificial Sequence
  SBL probe for Q61K mutation
gggtcatatcggtcactgttgtactcctcvtg

SEQ ID NO: 18 - DNA, 32 bp, Artificial Sequence
  SBL probe for Q61K mutation
gggtcatatcggtcactgttgtactcctctvg

SEQ ID NO: 19 - DNA, 32 bp, Artificial Sequence
  SBL probe for Q61K mutation
gggtcatatcggtcactgttgtactcctctth

SEQ ID NO: 20 - DNA, 12 bp, Artificial Sequence
  SBL probe for Q61K mutation
gtactcctcttg

SEQ ID NO: 21 - DNA, 41 bp, Artificial Sequence
  SBL primer for A146P mutation
tgatgtttcaataaaaggaatcaggatacacactacccgtg

SEQ ID NO: 22 - DNA, 32 bp, Artificial Sequence
  SBL probe for A146P mutation
  misc_feature (30)..(32): n is a, c, g, or t
gggtcatatcggtcactgtttcttgtcttnnn

SEQ ID NO: 23 - DNA, 32 bp, Artificial Sequence
  SBL probe for A146P mutation
gggtcatatcggtcactgtttcttgtcttvgc

SEQ ID NO: 24 - DNA, 32 bp, Artificial Sequence
  SBL probe for A146P mutation
gggtcatatcggtcactgtttcttgtcttthc

SEQ ID NO: 25 - DNA, 32 bp, Artificial Sequence
  SBL probe for A146P mutation
gggtcatatcggtcactgtttcttgtctttgd

SEQ ID NO: 26 - DNA, 12 bp, Artificial Sequence
  SBL probe for A146P mutation
tcttgtctttgc

SEQ ID NO: 27 - DNA, 40 bp, Artificial Sequence
  SBL primer for G13C mutation
accagctccaactaccacaacaggatacacactacccgtg

SEQ ID NO: 28 - DNA, 32 bp, Artificial Sequence
  SBL probe for G13C mutation
  misc_feature (30)..(32): n is a, c, g, or t
gggtcatatcggtcactgttcttgcctacnnn

SEQ ID NO: 29 - DNA, 32 bp, Artificial Sequence
  SBL probe for G13C mutation
gggtcatatcggtcactgttcttgcctacdgg

SEQ ID NO: 30 - DNA, 32 bp, Artificial Sequence
  SBL probe for G13C mutation
gggtcatatcggtcactgttcttgcctacchg

SEQ ID NO: 31 - DNA, 32 bp, Artificial Sequence
  SBL probe for G13C mutation
gggtcatatcggtcactgttcttgcctaccgh

SEQ ID NO: 32 - DNA, 12 bp, Artificial Sequence
  SBL probe for G13C mutation
cttgcctaccgg

SEQ ID NO: 33 - DNA, 40 bp, Artificial Sequence; identical to SEQ ID NO: 9
  SBL primer for G12,13 (adjacent) mutation

SEQ ID NO: 34 - DNA, 32 bp, Artificial Sequence
  SBL probe for G12,13 (adjacent) mutation
  misc_feature (27)..(32): n is a, c, g, or t
gggtcatatcggtcactgttgcctacnnnnnn

SEQ ID NO: 35 - DNA, 32 bp, Artificial Sequence
  SBL probe for G12,13 (adjacent) mutation
gggtcatatcggtcactgttgcctachccacc

SEQ ID NO: 36 - DNA, 32 bp, Artificial Sequence
  SBL probe for G12,13 (adjacent) mutation
gggtcatatcggtcactgttgcctacgdcacc

SEQ ID NO: 37 - DNA, 32 bp, Artificial Sequence
  SBL probe for G12,13 (adjacent) mutation
gggtcatatcggtcactgttgcctacgcdacc

SEQ ID NO: 38 - DNA, 32 bp, Artificial Sequence; identical to SEQ ID NO: 11
  SBL probe for G12,13 (adjacent) mutation

SEQ ID NO: 39 - DNA, 32 bp, Artificial Sequence; identical to SEQ ID NO: 12
  SBL probe for G12,13 (adjacent) mutation

SEQ ID NO: 40 - DNA, 32 bp, Artificial Sequence; identical to SEQ ID NO: 13
  SBL probe for G12,13 (adjacent) mutation

SEQ ID NO: 41 - DNA, 12 bp, Artificial Sequence
  SBL probe for G12,13 (adjacent) mutation
gcctacgccacc

SEQ ID NO: 42 - DNA, 40 bp, Artificial Sequence
  SBL primer of synthetic transcript
agatgggacctacaatgtaccagaagcgtctatgacactc

SEQ ID NO: 43 - DNA, 20 bp, Artificial Sequence
  SBL primer of synthetic transcript
cgatcgtctacttctattga

SEQ ID NO: 44 - DNA, 28 bp, Artificial Sequence
  SBL primer of synthetic transcript
cgatcgatcgatcgtctacttctattgc

SEQ ID NO: 45 - DNA, 35 bp, Artificial Sequence
  SBL primer of synthetic transcript
gatcgatcgatcgatcgatcgtctacttctattgg

SEQ ID NO: 46 - DNA, 42 bp, Artificial Sequence
  SBL primer of synthetic transcript
atcgatcgatcgatcgatcgatcgatcgtctacttctattgt

SEQ ID NO: 47 - RNA, 41 nt, Artificial Sequence
  Synthetic RNA transcript
gacgcuucugguacauuguaggucccaucuucaauagaagu

SEQ ID NO: 48 - DNA, 20 bp, Artificial Sequence
  QPCR primer AP1
gggtcatatcggtcactgtt

SEQ ID NO: 49 - DNA, 20 bp, Artificial Sequence
  QPCR primer AP2
cacgggtagtgtgtatcctg

SEQ ID NO: 50 - DNA, 32 bp, Artificial Sequence
  Forward sequencing primer (IDT)
  misc_feature (21)..(32): n is a, c, g, or t
gggtcatatcggtcactgttnnnnnnnnnnnn

SEQ ID NO: 51 - DNA, 40 bp, Homo sapiens
agcagctgtgtccatgcagccaggatacacactacccgtg

SEQ ID NO: 52 - DNA, 40 bp, Homo sapiens
atgtttgtgatgtgcccggtcaggatacacactacccgtg

SEQ ID NO: 53 - DNA, 40 bp, Homo sapiens
aaacataatcctcaggtatccaggatacacactacccgtg

SEQ ID NO: 54 - DNA, 40 bp, Homo sapiens
aatgtgctttcaattgatggcaggatacacactacccgtg

SEQ ID NO: 55 - DNA, 40 bp, Homo sapiens
tactgttcttgaaatacctacaggatacacactacccgtg

SEQ ID NO: 56 - DNA, 40 bp, Homo sapiens
acacacaattcatcaagcaccaggatacacactacccgtg

SEQ ID NO: 57 - DNA, 40 bp, Homo sapiens
ttcctgaggttctgcatccacaggatacacactacccgtg

SEQ ID NO: 58 - DNA, 40 bp, Homo sapiens
attctttccagcattgtgctcaggatacacactacccgtg

SEQ ID NO: 59 - DNA, 40 bp, Homo sapiens
tgaccccaataaagtttctgcaggatacacactacccgtg

SEQ ID NO: 60 - DNA, 40 bp, Homo sapiens
agcactgaggtgggggagcacaggatacacactacccgtg

SEQ ID NO: 61 - DNA, 40 bp, Homo sapiens
acagtgaagacaagaatggtcaggatacacactacccgtg

SEQ ID NO: 62 - DNA, 40 bp, Homo sapiens
tttgagctttgaacactgaacaggatacacactacccgtg

SEQ ID NO: 63 - DNA, 40 bp, Homo sapiens
aaagaaaaacccaatgtaaacaggatacacactacccgtg

SEQ ID NO: 64 - DNA, 40 bp, Homo sapiens
ttatttccaaatacttgagacaggatacacactacccgtg

SEQ ID NO: 65 - DNA, 40 bp, Homo sapiens
aaaaccacagataaccaaggcaggatacacactacccgtg

SEQ ID NO: 66 - DNA, 40 bp, Homo sapiens
tttagaagaatgcctcctcgcaggatacacactacccgtg

SEQ ID NO: 67 - DNA, 40 bp, Homo sapiens
ttgtactaacttatgatagacaggatacacactacccgtg

SEQ ID NO: 68 - DNA, 40 bp, Homo sapiens
aaggagcgggtgtgggagaacaggatacacactacccgtg

SEQ ID NO: 69 - DNA, 40 bp, Homo sapiens
agctgtgggcacattatgtacaggatacacactacccgtg

SEQ ID NO: 70 - DNA, 40 bp, Homo sapiens
agtcctttgaggggacagatcaggatacacactacccgtg

SEQ ID NO: 71 - DNA, 40 bp, Homo sapiens
tgcctgcagccttcataagccaggatacacactacccgtg

SEQ ID NO: 72 - DNA, 40 bp, Homo sapiens
agccatggagtgggcactaccaggatacacactacccgtg

SEQ ID NO: 73 - DNA, 40 bp, Homo sapiens
tcggttttgccggtttctttcaggatacacactacccgtg

SEQ ID NO: 74 - DNA, 40 bp, Homo sapiens
acgcttcaatttccttccatcaggatacacactacccgtg

SEQ ID NO: 75 - DNA, 40 bp, Homo sapiens
aagctgccaaagaacacatccaggatacacactacccgtg

SEQ ID NO: 76 - DNA, 40 bp, Homo sapiens
atgtcatgaaagattttgagcaggatacacactacccgtg

SEQ ID NO: 77 - DNA, 40 bp, Homo sapiens
tctgtccatgatgtcaaaagcaggatacacactacccgtg

SEQ ID NO: 78 - DNA, 40 bp, Homo sapiens
tgtacactgaggtaggaaatcaggatacacactacccgtg

SEQ ID NO: 79 - DNA, 40 bp, Homo sapiens
agcgcttggtctgtgtccttcaggatacacactacccgtg

SEQ ID NO: 80 - DNA, 40 bp, Homo sapiens
tagctgcgaccaacaacagccaggatacacactacccgtg

SEQ ID NO: 81 - DNA, 40 bp, Homo sapiens
aatcataaaataacagtaaacaggatacacactacccgtg

SEQ ID NO: 82 - DNA, 40 bp, Homo sapiens
tcatccccaagcccctcaagcaggatacacactacccgtg

SEQ ID NO: 83 - DNA, 40 bp, Homo sapiens
accctcttaaatgtcaaagacaggatacacactacccgtg

SEQ ID NO: 84 - DNA, 40 bp, Homo sapiens
ttctcatcctttctcgctctcaggatacacactacccgtg

SEQ ID NO: 85 - DNA, 40 bp, Homo sapiens
tgccctgcacattttcttttcaggatacacactacccgtg

SEQ ID NO: 86 - DNA, 40 bp, Homo sapiens
aagtaaagaaacgctttgtacaggatacacactacccgtg

SEQ ID NO: 87 - DNA, 40 bp, Homo sapiens
aaagtgtcatcaatttgtaacaggatacacactacccgtg

SEQ ID NO: 88 - DNA, 40 bp, Homo sapiens
agcttgggagccacatggctcaggatacacactacccgtg

SEQ ID NO: 89 - DNA, 40 bp, Homo sapiens
agcttgatcaccaccgccttcaggatacacactacccgtg

SEQ ID NO: 90 - DNA, 40 bp, Homo sapiens
tggccttcagaagcatctcccaggatacacactacccgtg

SEQ ID NO: 91 - DNA, 40 bp, Homo sapiens
aaatcacctgcagctaagcacaggatacacactacccgtg

SEQ ID NO: 92 - DNA, 40 bp, Homo sapiens
aagtgggctttttgtgaactcaggatacacactacccgtg

SEQ ID NO: 93 - DNA, 40 bp, Homo sapiens
tggcagcagggcctcgggaacaggatacacactacccgtg

SEQ ID NO: 94 - DNA, 40 bp, Homo sapiens
agaattaaaaagctttttttcaggatacacactacccgtg

SEQ ID NO: 95 - DNA, 40 bp, Homo sapiens
ttaacttgcccaaagttcaccaggatacacactacccgtg

SEQ ID NO: 96 - DNA, 40 bp, Homo sapiens
aaaaagttgtaatagagaatcaggatacacactacccgtg

SEQ ID NO: 97 - DNA, 40 bp, Homo sapiens
tccttgatgtcaaaatggggcaggatacacactacccgtg

SEQ ID NO: 98 - DNA, 40 bp, Homo sapiens
agacccacccataaggctgccaggatacacactacccgtg

SEQ ID NO: 99 - DNA, 40 bp, Homo sapiens
acgccttctccaacaaaagacaggatacacactacccgtg

SEQ ID NO: 100 - DNA, 40 bp, Homo sapiens
accaacagaccccaatttcccaggatacacactacccgtg

SEQ ID NO: 101 - DNA, 40 bp, Homo sapiens
atcgggaccgagacctgcttcaggatacacactacccgtg

SEQ ID NO: 102 - DNA, 40 bp, Homo sapiens
tttttaagagcaaattctgtcaggatacacactacccgtg

SEQ ID NO: 103 - DNA, 40 bp, Homo sapiens
ttagagatagagaaacagaccaggatacacactacccgtg

SEQ ID NO: 104 - DNA, 40 bp, Homo sapiens
ttggtagtcatgtctttgtgcaggatacacactacccgtg

SEQ ID NO: 105 - DNA, 40 bp, Homo sapiens
ttcttctctgctgcatttggcaggatacacactacccgtg

SEQ ID NO: 106 - DNA, 40 bp, Homo sapiens
tcgctgtgtgaatgggcagtcaggatacacactacccgtg

SEQ ID NO: 107 - DNA, 40 bp, Homo sapiens
acagagatgcagatggacggcaggatacacactacccgtg

SEQ ID NO: 108 - DNA, 40 bp, Homo sapiens
tgatgtccacactgctcggccaggatacacactacccgtg

SEQ ID NO: 109 - DNA, 40 bp, Homo sapiens
aaagtggtgaggagaaaacacaggatacacactacccgtg

SEQ ID NO: 110 - DNA, 40 bp, Homo sapiens
tgatggaagcagcggaggcccaggatacacactacccgtg

SEQ ID NO: 111 - DNA, 40 bp, Homo sapiens
agagcaaggctgtagagattcaggatacacactacccgtg

SEQ ID NO: 112 - DNA, 40 bp, Homo sapiens
aactaaatcccgaaatacaacaggatacacactacccgtg

SEQ ID NO: 113 - DNA, 40 bp, Homo sapiens
aggatagcctttcagaccaacaggatacacactacccgtg

SEQ ID NO: 114 - DNA, 40 bp, Homo sapiens
acgccccacctgccaccctccaggatacacactacccgtg

SEQ ID NO: 115 - DNA, 40 bp, Homo sapiens
agcactaccatgcagggtaccaggatacacactacccgtg

SEQ ID NO: 116 - DNA, 40 bp, Homo sapiens
taaaagtgtaataatggaaacaggatacacactacccgtg

SEQ ID NO: 117 - DNA, 40 bp, Homo sapiens
tgctcgcagtgggctgattccaggatacacactacccgtg

SEQ ID NO: 118 - DNA, 40 bp, Homo sapiens
aggggagtgatttaagcaatcaggatacacactacccgtg

SEQ ID NO: 119 - DNA, 40 bp, Homo sapiens
attctagagtttggaatgcacaggatacacactacccgtg

SEQ ID NO: 120 - DNA, 40 bp, Homo sapiens
actgtgctaggctatccaagcaggatacacactacccgtg

SEQ ID NO: 121 - DNA, 40 bp, Homo sapiens
aaagtttataaacaaagctccaggatacacactacccgtg

SEQ ID NO: 122 - DNA, 40 bp, Homo sapiens
acactcaccttagggttcagcaggatacacactacccgtg

SEQ ID NO: 123 - DNA, 40 bp, Homo sapiens
ttgacatcaagaaaagactacaggatacacactacccgtg

SEQ ID NO: 124 - DNA, 40 bp, Homo sapiens
atccactggtactgcaggttcaggatacacactacccgtg

SEQ ID NO: 125 - DNA, 40 bp, Homo sapiens
tgactggcccacacgtgcatcaggatacacactacccgtg

SEQ ID NO: 126 - DNA, 40 bp, Homo sapiens
aaccctgggtcaaaaagagacaggatacacactacccgtg

SEQ ID NO: 127 - DNA, 40 bp, Homo sapiens
agtaggtgtgggtaatttggcaggatacacactacccgtg

SEQ ID NO: 128 - DNA, 40 bp, Homo sapiens
aggatcagaaacagctttggcaggatacacactacccgtg

SEQ ID NO: 129 - DNA, 40 bp, Homo sapiens
agtgctttgtagtctctcctcaggatacacactacccgtg

SEQ ID NO: 130 - DNA, 40 bp, Homo sapiens
tccctgacactttaacctcacaggatacacactacccgtg

SEQ ID NO: 131 - DNA, 40 bp, Homo sapiens
tagacacattctttattattcaggatacacactacccgtg

SEQ ID NO: 132 - DNA, 40 bp, Homo sapiens
ttctcagtgtccacagcgcacaggatacacactacccgtg

SEQ ID NO: 133 - DNA, 40 bp, Homo sapiens
tttaatatgcttataacctacaggatacacactacccgtg

SEQ ID NO: 134 - DNA, 40 bp, Homo sapiens
ttaaagaccctgagttatgtcaggatacacactacccgtg

SEQ ID NO: 135 - DNA, 40 bp, Homo sapiens
aacttcttaccctaaaagagcaggatacacactacccgtg

SEQ ID NO: 136 - DNA, 40 bp, Homo sapiens
ttttaaaatactagcctgtacaggatacacactacccgtg

SEQ ID NO: 137 - DNA, 40 bp, Homo sapiens
aaactcaggaaaacctctcacaggatacacactacccgtg

SEQ ID NO: 138 - DNA, 40 bp, Homo sapiens
ttctctcccgtatcacctaacaggatacacactacccgtg

SEQ ID NO: 139 - DNA, 40 bp, Homo sapiens
aaaaaaaaaatggaaaaaaacaggatacacactacccgtg

SEQ ID NO: 140 - DNA, 40 bp, Homo sapiens
aaaaacaagctggataaccacaggatacacactacccgtg

SEQ ID NO: 141 - DNA, 40 bp, Homo sapiens
tggcttgtcttttttcaccacaggatacacactacccgtg

SEQ ID NO: 142 - DNA, 40 bp, Homo sapiens
tctcagtgtttcaacctcaccaggatacacactacccgtg

SEQ ID NO: 143 - DNA, 40 bp, Homo sapiens
aaagtctcgtgcacatgtgacaggatacacactacccgtg

SEQ ID NO: 144 - DNA, 40 bp, Homo sapiens
tgatggtgctcagattgcaacaggatacacactacccgtg

SEQ ID NO: 145 - DNA, 40 bp, Homo sapiens
tgcaacaaaagattagaacacaggatacacactacccgtg

SEQ ID NO: 146 - DNA, 40 bp, Homo sapiens
ttagtatatagaaataataccaggatacacactacccgtg

SEQ ID NO: 147 - DNA, 44 bp, Artificial Sequence
  SBL primer for KRAS G12D mutation for in situ RCA
agctccaaccaccacaagtttatactcagtcattttctcgggaa

SEQ ID NO: 148 - DNA, 18 bp, Artificial Sequence
  SBL probe for KRAS G12V mutation for in situ RCA
cgctgaagatacgccacc

SEQ ID NO: 149 - DNA, 18 bp, Artificial Sequence
  SBL probe for KRAS G12V mutation for in situ RCA
cgctgaagatacgccadc

SEQ ID NO: 150 - DNA, 18 bp, Artificial Sequence
  SBL probe for KRAS G12V mutation for in situ RCA
cgctgaagatacgccacd

SEQ ID NO: 151 - DNA, 18 bp, Artificial Sequence
  SBL probe for KRAS G12V mutation for in situ RCA
cgctgaagagcctacgcc

SEQ ID NO: 152 - DNA, 37 bp, Artificial Sequence
  SBL primer for TP53 R273H mutation for in situ RCA
cacctcaaagctgttccgtcccagtagatctcgggaa

SEQ ID NO: 153 - DNA, 19 bp, Artificial Sequence
  SBL probe for TP53 R273C mutation for in situ RCA
cgctgaagacacaaacacg

SEQ ID NO: 154 - DNA, 19 bp, Artificial Sequence
  SBL probe for TP53 R273C mutation for in situ RCA
cgctgaagacacaaacadg

SEQ ID NO: 155 - DNA, 19 bp, Artificial Sequence
  SBL probe for TP53 R273C mutation for in situ RCA
cgctgaagacacaaacach

SEQ ID NO: 156 - DNA, 19 bp, Artificial Sequence
  SBL probe for TP53 R273C mutation for in situ RCA
cgctgaagaaggcacaaac

SEQ ID NO: 157 - DNA, 35 bp, Artificial Sequence
  SBL primer for TP53 T220C mutation for in situ RCA
gggcaccaccacactatgtcgaaaagtctcgggaa

SEQ ID NO: 158 - DNA, 19 bp, Artificial Sequence
  SBL probe for TP53 T220C mutation for in situ RCA
cgctgaagagcggctcata

SEQ ID NO: 159 - DNA, 19 bp, Artificial Sequence
  SBL probe for TP53 T220C mutation for in situ RCA
cgctgaagagcggctcava

SEQ ID NO: 160 - DNA, 19 bp, Artificial Sequence
  SBL probe for TP53 T220C mutation for in situ RCA
cgctgaagagcggctcatb

SEQ ID NO: 161 - DNA, 19 bp, Artificial Sequence
  SBL probe for TP53 T220C mutation for in situ RCA
cgctgaagacaggcggctc

SEQ ID NO: 162 - DNA, 42 bp, Artificial Sequence
  Sequencing primer for ProRSBL nucleotide bias determination
cgatgggacctacaatgtaccagaagcgtctctatgacactc

SEQ ID NO: 163 - DNA, 44 bp, Artificial Sequence
  Sequencing primer for ProRSBL nucleotide bias determination
ggatgggacctacaatgtaccagaagcgtctctctatgacactc

SEQ ID NO: 164 - DNA, 46 bp, Artificial Sequence
  Sequencing primer for ProRSBL nucleotide bias determination
tgatgggacctacaatgtaccagaagcgtctctctctatgacactc

SEQ ID NO: 165 - DNA, 20 bp, Artificial Sequence
  Competitive probe for ProRSBL nucleotide bias determination
cgatcgtctacttctattga

SEQ ID NO: 166 - DNA, 28 bp, Artificial Sequence
  Competitive probe for ProRSBL nucleotide bias determination
cgatcgatcgatcgtctacttctattgc

SEQ ID NO: 167 - DNA, 36 bp, Artificial Sequence
  RNA template for ProRSBL nucleotide bias determination
cgatcgatcgatcgatcgatcgtctacttctattgg

SEQ ID NO: 168 - RNA, 42 nt, Artificial Sequence
  RNA template for ProRSBL nucleotide bias determination
gacgcuucugguacauuguaggucccaucugcaauagaagua

SEQ ID NO: 169 - RNA, 42 nt, Artificial Sequence
  RNA template for ProRSBL nucleotide bias determination
gacgcuucugguacauuguaggucccaucuccaauagaagua

SEQ ID NO: 170 - RNA, 42 nt, Artificial Sequence
  RNA template for ProRSBL nucleotide bias determination
gacgcuucugguacauuguaggucccaucuacaauagaagua

SEQ ID NO: 171 - RNA, 42 nt, Artificial Sequence
  RNA template for ProRSBL nucleotide bias determination
gacgcuucugguacauuguaggucccaucaucaauagaagua

SEQ ID NO: 172 - RNA, 42 nt, Artificial Sequence
  RNA template for ProRSBL nucleotide bias determination
gacgcuucugguacauuguaggucccaucagcaauagaagua

SEQ ID NO: 173 - RNA, 42 nt, Artificial Sequence
  RNA template for ProRSBL nucleotide bias determination
gacgcuucugguacauuguaggucccaucaccaauagaagua

SEQ ID NO: 174 - RNA, 42 nt, Artificial Sequence
  RNA template for ProRSBL nucleotide bias determination
gacgcuucugguacauuguaggucccaucaacaauagaagua

SEQ ID NO: 175 - RNA, 42 nt, Artificial Sequence
  RNA template for ProRSBL nucleotide bias determination
gacgcuucugguacauuguaggucccaucgucaauagaagua

SEQ ID NO: 176 - RNA, 42 nt, Artificial Sequence
  RNA template for ProRSBL nucleotide bias determination
gacgcuucugguacauuguaggucccaucggcaauagaagua

SEQ ID NO: 177 - RNA, 42 nt, Artificial Sequence
  RNA template for ProRSBL nucleotide bias determination
gacgcuucugguacauuguaggucccaucgccaauagaagua

SEQ ID NO: 178 - RNA, 42 nt, Artificial Sequence
  RNA template for ProRSBL nucleotide bias determination
gacgcuucugguacauuguaggucccaucgacaauagaagua

SEQ ID NO: 179 - RNA, 42 nt, Artificial Sequence
  RNA template for ProRSBL nucleotide bias determination
gacgcuucugguacauuguaggucccauccucaauagaagua

SEQ ID NO: 180 - RNA, 42 nt, Artificial Sequence
  RNA template for ProRSBL nucleotide bias determination
gacgcuucugguacauuguaggucccauccgcaauagaagua

SEQ ID NO: 181 - RNA, 42 nt, Artificial Sequence
  RNA template for ProRSBL nucleotide bias determination
gacgcuucugguacauuguaggucccauccccaauagaagua

SEQ ID NO: 182 - RNA, 42 nt, Artificial Sequence
  RNA template for ProRSBL nucleotide bias determination
gacgcuucugguacauuguaggucccauccacaauagaagua

SEQ ID NO: 183 - RNA, 42 nt, Artificial Sequence
  Reverse sequencing RNA
gacgauucugguucauuguaggucccaucuucaauagaagua

SEQ ID NO: 184 - DNA, 63 bp, Artificial Sequence
  Forwarding sequencing primer
agatgggacctacaatgtaccagaagcgtctatgacactcgatcaggatacacactacccgtg

SEQ ID NO: 185 - DNA, 62 bp, Artificial Sequence
  Reverse sequencing primer
ttgggtcatatcggtcactgtttatgacactctacttctattgaagatgggacctacaatga

SEQ ID NO: 186 - DNA, 42 bp, Artificial Sequence
  Forward ProRSBL probe 1-4
  misc_feature (39)..(42): n is a, c, g, or t
ttgggtcatatcggtcactgttcgatcgtctacttctannnn

SEQ ID NO: 187 - DNA, 42 bp, Artificial Sequence
  Forward ProRSBL probe 5-8
  misc_feature (35)..(38): n is a, c, g, or t
ttgggtcatatcggtcactgttcgatcgtctactnnnnttga

SEQ ID NO: 188 - DNA, 42 bp, Artificial Sequence
  Forward ProRSBL probe 9-12
  misc_feature (31)..(34): n is a, c, g, or t
ttgggtcatatcggtcactgttcgatcgtcnnnntctattga

SEQ ID NO: 189 - DNA, 43 bp, Artificial Sequence
  Reverse ProRSBL probe 1-4
  misc_feature (1)..(4): n is a, c, g, or t
nnnngaatcgtccgatcgtcgatcaggatacacactacccgtg

SEQ ID NO: 190 - DNA, 43 bp, Artificial Sequence
  Reverse ProRSBL probe 9-12
  misc_feature (5)..(8): n is a, c, g, or t
accannnncgtccgatcgtcgatcaggatacacactacccgtg

SEQ ID NO: 191 - DNA, 42 bp, Artificial Sequence
  Reverse ProRSBL probe 9-12
  misc_feature (9)..(12): n is a, c, g, or t
accagaatnnnncgatcgtcgatcaggatacacactacccgg

SEQ ID NO: 192 - RNA, 52 nt, Artificial Sequence
  Synthetic RNA (from human 28S rRNA 4490-4540)
cgugagcuggguuuagaccgucgugagacagguuaguuuuacccuacugaug

SEQ ID NO: 193 - DNA, 40 bp, Artificial Sequence
  Cleavage substrate I3 (for cleavage kinetics)
gtaaaacaacctgtctcacgacggtctaaacccagctcac

SEQ ID NO: 194 - DNA, 29 bp, Artificial Sequence
  Cleavage substrate I8 (for post cleavage ligation)
gctagctagctagctagcaggtaaaacta

SEQ ID NO: 195 - DNA, 31 bp, Artificial Sequence
  ligation probe A
tagctagctagctagctagcaggtaaaactc

SEQ ID NO: 196 - DNA, 33 bp, Artificial Sequence
  ligation probe C
gctagctagctagctagctagcaggtaaaactg

SEQ ID NO: 197 - DNA, 33 bp, Artificial Sequence; identical to SEQ ID NO: 196
  ligation probe G

SEQ ID NO: 198 - DNA, 35 bp, Artificial Sequence
  ligation probe T
ctgctagctagctagctagctagcaggtaaaactt

SEQ ID NO: 199 - DNA, 43 bp, Artificial Sequence
  SBL primer for IDH1 mutant detection
cctatgatgataggttttacccatccactcacaatctcgggaa

SEQ ID NO: 200 - DNA, 19 bp, Artificial Sequence
  SBL probe for IDH1 mutant detection
cgctgaagaaagcatgach

SEQ ID NO: 201 - DNA, 19 bp, Artificial Sequence
  SBL probe for IDH1 mutant detection
cgctgaagaaagcatgadg

SEQ ID NO: 202 - DNA, 19 bp, Artificial Sequence
  SBL probe for IDH1 mutant detection
cgctgaagaaagcatgbcg

SEQ ID NO: 203 - DNA, 19 bp, Artificial Sequence
  SBL probe for IDH1 mutant detection
cgctgaagaaagcatgacg

SEQ ID NO: 204 - RNA, 32 nt, Artificial Sequence
  Synthetic template for KRAS mutant detection
aacuugugguaguuggagcugguggcguaggc

SEQ ID NO: 205 - RNA, 32 nt, Artificial Sequence
  Synthetic template for KRAS mutant detection
aacuugugguaguuggagcugauggcguaggc

SEQ ID NO: 206 - RNA, 32 nt, Artificial Sequence
  Synthetic template for KRAS mutant detection
aacuugugguaguuggagcuguuggcguaggc

SEQ ID NO: 207 - RNA, 32 nt, Artificial Sequence
  Synthetic template for KRAS mutant detection
aacuugugguaguuggagcucguggcguaggc

SEQ ID NO: 208 - DNA, 55 bp, Artificial Sequence
  SBL primer for KRAS mutant detection
agctccaaccaccacaagtttatactcagtcatttcaggatacacactacccgtg

SEQ ID NO: 209 - DNA, 29 bp, Artificial Sequence
  SBL probe for KRAS mutant detection
gggtcatatcggtcactgtttacgccacd

SEQ ID NO: 210 - DNA, 29 bp, Artificial Sequence
  SBL probe for KRAS mutant detection
gggtcatatcggtcactgtttacgccadc

SEQ ID NO: 211 - DNA, 29 bp, Artificial Sequence
  SBL probe for KRAS mutant detection
gggtcatatcggtcactgtttacgccbcc

SEQ ID NO: 212 - DNA, 12 bp, Artificial Sequence
  SBL probe
gcctacgccacd

SEQ ID NO: 213 - DNA, 12 bp, Artificial Sequence
  SBL Probe
gcctacgccadc

(19) United States
(12) Patent Application Publication
(10) Pub. No.: US 2022/0042090 A1
(43) Pub. Date: Feb. 10, 2022

(54) PROGRAMMABLE RNA-TEMPLATED SEQUENCING BY LIGATION (rSBL)

(71) Applicant: Cold Spring Harbor Laboratory, Cold Spting Harbor, NY (US)

(72) Inventors: Je H. Lee, Huntington, NY (US); David Chitty, Long Island City, NY (US); Ahmed Elewa, Cold Spring Harbor, NY (US); Debarati Ghosh, Hicksville, NY (US); Simone Weinmann, Northport, NY (US); Daniel Fuerth, New York, NY (US); Chengxiang Yuan, Cold Spring Harbor, NY (US)

(73) Assignee: Cold Spring Harbor Laboratory, Cold Spring Harbor, NY (US)

(21) Appl. No.: 17/275,928

(22) PCT Filed: Sep. 13, 2019
(86) PCT No.: PCT/US2019/051184

§ 371 Date: Mar. 12, 2021

Related U.S. Application Data
(60) Provisional application No. 62/731,708, filed on Sep. 14, 2018.

(51) Int. Cl. C12Q 1/6869; C12Q 1/6827; C12Q 1/6883

(52) U.S. Cl. CPC C12Q 1/6869; C12Q 2600/156; C12Q 1/6883; C12Q 1/6827

(57) ABSTRACT

A method for determining the presence or absence of variant ribonucleic acid molecules in a population of ribonucleic acid molecules, wherein the reference sequence of the variant ribonucleic acid molecules is known, the method comprising: (a) interrogating the population of ribonucleic acid molecules with a plurality of primer molecules and a plurality of probes so as to saturate the population of ribonucleic acid molecules with the probes and primer molecules such that the probes and primer molecules are adjacent to one another when hybridized to their respective complimentary sequences on the ribonucleic acid molecules, (b) ligating the probes to their respective adjacent primer molecules so as to form ligated nucleic acid molecules, and (c) detecting the presence of ligated nucleic acid molecules that are complementary to a sequence that differs from the reference sequence, thereby determining the presence or absence of one or more variant ribonucleic acid molecules in the population of ribonucleic acid molecules.

This application claims priority of U.S. Provisional Application No. 62/731,708, filed Sep. 14, 2018, the contents of which are hereby incorporated by reference.

Throughout this application, various publications are referenced, including referenced in parenthesis. The disclosures of all publications mentioned in this application in their entireties are hereby incorporated by reference into this application in order to more fully describe the state of the art to which this invention pertains.

### REFERENCE TO SEQUENCE LISTING

This application incorporates-by-reference nucleotide sequences which are present in the file named “190913_90418-A-PCT_Sequence_Listing_DH.txt”, which is 41 kilobytes in size, and which was created on Sep. 13, 2019 in the IBM-PC machine format, having an operating system compatibility with MS-Windows, which is contained in the text file filed Sep. 13, 2019 as part of this application.

### BACKGROUND OF INVENTION

Cells in the human body accumulate hundreds of de novo mutations over the span of a lifetime, especially in frequently dividing cell types. While only a fraction of somatic mutations modifies the protein function, harmful variants eventually emerge and contribute to age-related disorders, including cancer. Therefore, timely detection of pathogenic protein alterations is an important goal in disease screening, early diagnosis, and treatment monitoring.

One strategy for detecting altered proteins is based on antibodies (i.e. FACS-fluorescence-activated cell sorting, IHC-immunohistochemistry, ELISA-enzyme-linked immunosorbent assay). However, antibodies against specific amino acid alterations are challenging to generate. An alternative solution is to find cellular biomarkers downstream of initiating mutations, but such biomarkers are often present in other cell types. Regardless, the low specificity of mutation-associated antibodies or biomarkers precludes multiplexed assays with a low false positive rate in early or residual disease detection.

Another strategy to sequence the DNA and infer their consequence on the protein in silico. In cancer research next-generation sequencing (NGS) has been instrumental in classifying cancer types based on high-frequency mutations or molecular signatures. In early or residual cancer, however, somatic mutations are present in a small number of cells, necessitating clonal expansion of single cells in vitro (i.e. organoids) or deep sequencing. But single-cell expansion is not an option for real-time disease assessment, and the cost of deep sequencing over a large patient population limits the number of genetic markers surveyed, reducing its diagnostic specificity and sensitivity (FIG. 14).

A less costly strategy involves allele-specific probes for PCR or droplet-based assays. Allele-specific primer technologies have been around for decades, and they can be relatively specific, robust, and affordable for a handful of mutations. However, the detection specificity varies from one locus to another, making it challenging to multiplex a large number of allele-specific probes. Furthermore, disease-causing base identity at each locus must be known in advance to design allele-specific primers, which is challenging for loci with numerous allelic combinations. Because of these limitations, the role of allele-specific PCR or droplet-based assays are not suited for profiling mutations across a large number of genetic loci (FIG. 14).

Among recent technologies, multiplexed single-molecule fluorescent in situ DNA or RNA hybridization (smFISH) could be a potential option for detecting somatic mutations in owing to its sensitivity, simplicity, and versatility. Allele-specific smFISH has been demonstrated as a proof-of-concept, but the single-base specificity is inadequate for clinical applications (FIG. 14). Multiplexed in situ RNA genotyping/sequencing (i.e. padlock probes) can overcome the single-base specificity limitation, however, the detection sensitivity is considerably lower compared to allele-specific smFISH, and in situ RNA genotyping/sequencing is difficult to implement due to its technical complexity. More importantly, disease-causing base identity at each locus must be known in advance to design allele-specific primers.

Latest advances in NGS technologies permit the quantification of rare DNA variants even in the blood sample (i.e. ‘liquid biopsy’), and a single-cell NGS workflow can profile somatic mutations from different tissue regions using microdissection. While these advances are conceptually important, they have not translated into readily deployable clinical assays for assessing early or residual disease. Key reasons include the lack of sensitivity and specificity largely due to the cost of deep sequencing across a large number of loci.

The high cost of deep sequencing is attributable to its sequence-agnostic nature. For example, one needs to sequence >10⁶ molecules in order to detect a single mutant molecule among 10⁶ wild-type DNA molecules. Therefore, sequencing 100 different loci requires sequencing 10⁸ reads on a single Illumina HiSeq lane. By extension, sequencing 1,000 loci with VAF of 10⁻⁸ across 100 patients could cost up to $100 million USD using NGS technologies. As a consequence, the sensitivity of clinical high-throughput sequencing is generally capped at 10⁻² VAF for practical reasons, which limits their utility in detecting early or residual disease.

In the example above, the cost of NGS reflects the disproportionate amount of unaltered sequences from ‘normal’ cells in the tissue sample. To overcome this bottleneck, antibody-dependent diseased cell sorting is often used to enrich for the variant sequence (FIG. 1B); however, this requires discovery and validation of specific biomarkers and cannot be generalized to all disease or cancer types. Alternatively, the depletion of unaltered sequences during NGS library construction can be used; however, most depletion strategies utilize DNA hybridization primers or Cas9 guide RNAs tuned to specific alleles. Because the single-base specificity is variable from one target to another, these methods require optimization for each locus (similar to issues faced by allele-specific PCR above), making the depletion of ‘normal’ sequences difficult across a large number of loci.

Regardless, NGS-based approaches do not address whether protein modifications are pertinent to the tissue of interest. In contrast, antibodies detect proteins that are actually expressed in disease-relevant tissues. However, antibodies lack the specificity for discriminating amino acid alterations. While RNA-seq can discriminate genetic mutations and quantify the level of gene expression pattern simultaneously, it has the same sensitivity limitation as other types of NGS applications (described above) due to the overwhelming abundance of wild-type transcripts that require deep-sequencing.

In summary, early or residual cancer screening requires the detection of functional mutations from numerous genetic loci amidst a large number of normal cells (FIG. 15). Antibodies or FISH probes can measure the expression level of proteins or transcripts, but most cannot detect small amino acid changes or mutations. NGS can detect thousands of genetic alterations de novo, but ultra-deep sequencing is often necessary to detect rare cancer cells or nucleic acids. For clinical applications, an entirely different approach is needed, where a large number of clinically relevant de novo mutations can be profiled using a low-cost assay. To determine the tissue-of-origin, such a platform should also be able to label, isolate, or visualize functional mutations directly in diseased cells or tissues (FIG. 15).

### SUMMARY OF THE INVENTION

The subject invention provides a method for determining the presence or absence of variant ribonucleic acid molecules in a population of ribonucleic acid molecules, wherein the reference sequence of the variant ribonucleic acid molecules is known, the method comprising:
(a) interrogating the population of ribonucleic acid molecules with:
   (i) a plurality of primer molecules comprising nucleotides, wherein:
      (1) at least 8 consecutive nucleotides, starting at the 5′ or the 3′ end of each primer molecule, are fully complimentary to consecutive nucleotides of the reference sequence, and
      (2) the primer molecules have a melting temperature of at least 50° C. when hybridized to their complimentary ribonucleic acid molecules in the population of ribonucleic acid molecules; and
   (ii) a plurality of probes comprising L+S nucleotides, wherein:
      (1) nucleotides L are complimentary to: (1) the reference sequence that is adjacent to the nucleotides of the reference sequence that each primer molecule is fully complimentary to, or (2) a sequence that differs from (1) at one or more nucleotide bases along the length of L.
      (2) nucleotides S are fully complementary to the reference sequence, and
      (3) L+S is 8 to 12 and L is at least 1,
   so as to saturate the population of ribonucleic acid molecules with the probes and primer molecules such that the probes and primer molecules are adjacent to one another when hybridized to their respective complimentary sequences on the ribonucleic acid molecules, wherein if the 5′ end of the primer molecules are adjacent to the 3′ end of the probes when both are hybridized to their respective complimentary sequences then at least 8 consecutive nucleotides at the 5′ end of the primer molecules are fully complimentary to nucleotides of the reference sequence and the primer molecules have a 5′ phosphorylated A or T, and wherein if the 5′ end of the probes are adjacent to the 3′ end of the primer molecules when both are hybridized to their respective complimentary sequences then at least 8 consecutive nucleotides at the 3′ end of the primer molecules are fully complimentary to nucleotides of the reference sequence and the probes have a 5′ phosphorylated A or T:
(b) ligating the probes to their respective adjacent primer molecules so as to form ligated nucleic acid molecules, wherein the probes are ligated in competition under conditions favoring the ligation of fully hybridized probes over partially hybridized probes, wherein such conditions comprise using a reaction temperature that is about the melting temperature of a probe of length L+S that is fully hybridized;
(c) detecting the presence of ligated nucleic acid molecules that are complementary to a sequence that differs from the reference sequence;
thereby determining the presence or absence of one or more variant ribonucleic acid molecules in the population of ribonucleic acid molecules.

The subject invention also provides a composition comprising a primer molecule and at least two probes,
(a) wherein the primer molecule and at least two probes are designed to hybridize to target sequences on a ribonucleic acid molecule such that:
   (i) the 5′ end of the primer molecule and the 3′ end of the at least two probes are adjacent when the probe and primer molecule are hybridized to their respective target sequences on the ribonucleic acid molecule; or
   (ii) the 5′ end of the at least two probes and the 3′ end of the primer molecule are adjacent when the probe and primer molecule are hybridized to their respective target sequences on the ribonucleic acid molecule;
(b) wherein the primer molecule:
   (i) has a melting temperature of at least 50° C. when hybridized to its target sequence;
   (ii) if the primer molecule is designed such that the 5′ end of the primer molecule and the 3′ end of the at least two probes are adjacent when the probe and primer molecule are hybridized to their respective target sequences on the ribonucleic acid molecule;
   (iii) comprises nucleotides starting at its 5′ end that are fully complimentary to at least 8 consecutive nucleotides of the target sequence and has a 5′ phosphorylated A or T if the primer molecule is designed such that the 5′ end of the primer molecule and the 3′ end of the at least two probes are adjacent when the probe and primer molecule are hybridized to their respective target sequence on the ribonucleic acid molecule; and
   (iv) comprises nucleotides starting at its 3′ end that are fully complimentary to at least 8 consecutive nucleotides of the target sequence if the primer molecule is designed such that the 3′ end of the primer molecule and the 5′ end of the at least two probes are adjacent when the probe and primer molecule are hybridized to their respective target sequence on the ribonucleic acid molecule:
(c) wherein the at least two probes:
   (i) comprise L+S nucleotides, wherein L+S is 8 to 12, and L is at least 1;
   (ii) differ in sequence from one another at only one nucleotide base along the length of L,
   (iii) are fully complementary to the target sequence of the probe along the length of S;
   (iv) have a 5′ phosphorylated A or T if the at least two probes are designed such that the 5′ end of the at least two probes and the 3′ end of the primer molecule are adjacent when the probes and primer molecule are hybridized to their respective target sequences on the ribonucleic acid molecule.

The subject invention also provides a kit comprising a primer molecule and at least two probes,
(a) wherein the primer molecule and at least two probes are designed to hybridize to target sequences on a ribonucleic acid molecule such that:
   (i) the 5′ end of the primer molecule and the 3′ end of the at least two probes are adjacent when the probe and primer molecule are hybridized to their respective target sequences on the ribonucleic acid molecule; or
   (ii) the 5′ end of the at least two probes and the 3′ end of the primer molecule are adjacent when the probe and primer molecule are hybridized to their respective target sequences on the ribonucleic acid molecule;
(b) wherein the primer molecule:
   (i) has a melting temperature of at least 50° C. when hybridized to its target sequence;
   (ii) if the primer molecule is designed such that the 5′ end of the primer molecule and the 3′ end of the at least two probes are adjacent when the probe and primer molecule are hybridized to their respective target sequences on the ribonucleic acid molecule;
   (iii) comprises nucleotides starting at its 5′ end that are fully complimentary to at least 8 consecutive nucleotides of the target sequence and has a 5′ phosphorylated A or T if the primer molecule is designed such that the 5′ end of the primer molecule and the 3′ end of the at least two probes are adjacent when the probe and primer molecule are hybridized to their respective target sequence on the ribonucleic acid molecule; and
   (iv) comprises nucleotides starting at its 3′ end that are fully complimentary to at least 8 consecutive nucleotides of the target sequence if the primer molecule is designed such that the 3′ end of the primer molecule and the 5′ end of the at least two probes are adjacent when the probe and primer molecule are hybridized to their respective target sequence on the ribonucleic acid molecule:
(c) wherein the at least two probes:
   (i) comprise L+S nucleotides, wherein L+S is 8 to 12, and L is at least 1;
   (ii) differ in sequence from one another at only one nucleotide base along the length of L,
   (iii) are fully complementary to the target sequence of the probe along the length of S;
   (iv) have a 5′ phosphorylated A or T if the at least two probes are designed such that the 5′ end of the at least two probes and the 3′ end of the primer molecule are adjacent when the probes and primer molecule are hybridized to their respective target sequences on the ribonucleic acid molecule.

The subject invention also provides a composition comprising complexes of primer molecules, probes and ribonucleic acid molecules,
(a) wherein the complexes comprise primer molecules and probes that are hybridized to target sequences on the ribonucleic acid molecules such that:
   (i) the 5′ end of the primer molecules and the 3′ end of the probes are adjacent when hybridized their respective target sequences on the ribonucleic acid molecules; or
   (ii) the 5′ end of the at least two probes and the 3′ end of the primer molecule are adjacent when hybridized their respective target sequences on the ribonucleic acid molecules;
(b) wherein the primer molecules:
   (i) have a melting temperature of at least 50° C. when hybridized to their target sequence;
   (ii) comprise nucleotides that are fully complimentary to at least 8 consecutive nucleotides of the target sequence at the 5′ end of the primer molecule and have a 5′ phosphorylated A or T if the primer molecules are hybridized to their target sequence on the ribonucleic acid molecules such that the 5′ end of the primer molecules and the 3′ end of the probes are adjacent; and
   (iii) comprise nucleotides that are fully complimentary to at least 8 consecutive nucleotides of the target sequence at the 3′ end of the primer molecule if the primer molecules are hybridized to their target sequence on the ribonucleic acid molecules such that the 3′ end of the primer molecules and the 5′ end of the probes are adjacent; and
(c) wherein the composition comprises at least two probes, wherein such probes:
   (i) comprise L+S nucleotides, wherein L+S is 8 to 12, and L is at least 1;
   (ii) differ in sequence from one another at only one nucleotide base along the length of L,
   (iii) are fully complementary to the target sequence of the at least two probes along the length of S;
   (iv) have a 5′ phosphorylated A or T if the probes are hybridized to their target sequence on the ribonucleic acid molecules such that the 3′ end of the primer molecules and the 5′ end of the probes are adjacent.

The subject invention also provides a method of treating a disease or condition associated with the presence of variant ribonucleic acid molecules in a subject, the method comprising:
(a) using the method of the invention to determine the presence or absence of a variant ribonucleic acid molecule in the subject;
(b) treating the subject based on the presence or absence of the variant ribonucleic acid molecule.

The present invention provides for characterizing individual cells by sequencing RNA directly without cDNA synthesis advances diagnostics and discovery. The present invention discloses a probe design that increases RNA templated ligation accuracy, enables multiple rounds of ligation and sequencing of mRNA variant classes without a priori knowledge of their exact sequences. The programmable sequencing chemistry permits cell characterization using conditional statements about single cells.

The subject invention also provides for the methods, processes, compositions, devices, and kits for practicing substantially what is shown and described.

### BRIEF DESCRIPTION OF THE DRAWINGS

FIGS. 1A-B: FIG. 1A shown a comparison of sequencing technology availability and utility. From simple and low-cost genotyping assays (e.g. PCR) to highly sensitive, comprehensive, and unbiased sequencing methods, the trade-off between the cost and the sensitivity of detecting genetic variants from wild-type sequences is a common theme, and is one of the main factors in determining the cost-effectiveness of individual methods for precision medicine or oncology. Beyond the cost barriers, currently it is difficult to identify early or disseminated cancer cells in tissues or in the bloodstream because they often lack cancer-specific surrogate markers. Therefore, characterizing these cells remains extraordinarily challenging, despite its importance to cancer prognosis and treatment. FIG. 1B shows a further comparison of sequencing technology and utility. While it is possible to enrich for cancer-associated genome for molecular profiling or analysis (i.e. genome or exome sequencing), it is difficult to identify rare cancer cells in tissues or in the bloodstream (i.e. circulating tumor cells or CTCs) because they could lack cancer-specific cell surface markers or biological features that could be utilized for cell labeling, isolation, or visualization (i.e. epithelial markers, cell proliferation markers). Therefore, characterizing these cells remains challenging, despite its importance to cancer prognosis and treatment.

FIGS. 2A-B: In FIG. 2A, utilizing non-targeted sequencing both wild type and mutant sequences are amplified and analyzed. Low-frequency mutations require many reads. The ratio of mutant (mut):wild type (wt) sequences becomes larger after exponential PCR requiring an even greater number of sequencing reads; FIG. 2B, utilizing rSBL (a method for sequencing or genotyping in intact cells), only non-wild type sequences whose identity is unknown are amplified. The identity of mutant sequences can then be determined by Sanger or NGS. If the sequencing product inside the cell also results in fluorescent or colorimetric label of single cells, rare cancer cells could be identified, isolated, and characterized in the absence of surrogate biomarkers, purely based on their genetic mutational signature.

FIGS. 3A-B: In traditional sequencing-by-ligation (SBL), for example, three mismatched interrogating probes (FAM, Cy3, and Cy5) and one correctly matched interrogating probe (TexR) compete for the same ligation site. The difference in their Tₘ is ˜1-2° C., enabling them to equilibrate freely depending on the reaction temperature. In FIG. 3A, three mismatched interrogating probes (FAM, Cy3, TexRed) and one correctly matched interrogating probe (Cy5) have different Tₘ temperatures. The level of probe hybridization is a function of probe Tₘ, which is a function of their length; While the slight difference in the probe melting temperature can be used to discriminate alleles (i.e. allele-specific PCR, allele-specific FISH), the fraction of correctly hybridized probes vary dramatically even with small changes in the reaction temperature; FIG. 3B, 50% of correct probes are hybridized while 10% of one-base mismatch probes are hybridized at the T_{reaction} shown. Many one-base mismatch probes are possible (3L where L is the length of the probe). However, the slight difference in the hybridization rate is insufficient to discriminate individual bases with high specificity, and it is variable for each locus. DNA ligases are slow to ligate nicked DNA strands, if it recognizes a base-pair mismatch. This dramatically improves the specificity of allele discrimination. The combination of competitive ligation, rapid equilibration of competing probes, and large difference between correct and mismatched probe ligation is key to sequencing-by-ligation.

FIG. 4: In SBL, the sequencing template hybridized to the sequencing primer is transiently bound to interrogating oligonucleotides of length L+S, in which S is additional non-degenerate bases complementary to T and L is unknown bases of interest.

FIGS. 5A-B: In FIG. 5A, near Tₘ eventually all T will be converted into TC with k₂ dominant. In FIG. 5B, below Tₘ some T will be trapped in the TI state, especially if I>>C. Since DNA ligases will eventually ligate any two adjacent oligos, including single-base mismatch pairs, the sequencing error rate rises when the reaction temperature is much less than Tₘ. The highest specificity will come from the reaction temperature >Tₘ, and the efficiency of the reaction is simply a function of the molecular concentration of various competing oligonucleotides and the ligase type.

FIGS. 6A-D: In FIG. 6A, PBCV DNA ligase performs RNA-splinted DNA ligation using one sequencing primer and competing short degenerate (full or partial) oligonucleotides. Highly degenerate probes could yield ligation products with one or more mismatches. To see whether we can cleave mismatch-containing DNA:RNA hybrids, T7 endonuclease I can be used against DNA:RNA (lane 5-8 of FIG. 6B) with or without (lane 1-4 of FIG. 6B) a single mismatch; FIG. 6B, electrophoresis gel following T7 endonuclease I digestion of RNA-splinted DNA ligation or SBL products. The ability of T7 endonuclease I to cut DNA on RNA is similar to that of DNA:DNA duplexes (right panel). T7 Endonuclease I does not cleave adapter sequencing handles used for PCR amplification.

In FIG. 6C, the resulting rSBL ligation product containing PCR primer adapters are amplified in vitro, producing a clean PCR band down to the single-molecule per cell amount. In FIG. 6D, rSBL is used for real-time qPCR to quantify the amount of RNA (while genotyping at the same time). Without T7 endo and exonucleases, the excess primers and un-ligated probes result in background amplification product (left panel), while the application of T7 endo and exonucleases results in quantitative PCR with higher sensitivity and specificity (right panel).

FIGS. 7A-C. FIG. 7A shows the experimental workflow for determining the absolute efficiency of rSBL ligation. FIG. 7B and FIG. 7C show that the 5′ phosphorylated base of the sequencing primer is critical. If it is C or G, the ligation efficiency by PBCV DNA ligase drops to <50%; therefore, it is critical to fix the 5′ moiety as A or T for consistent and efficient SBL. For genotyping applications, this means that the nucleotide variant of interest has to have A or T nearby, depending on the read length.

FIGS. 8A-C: FIG. 8A, SBL ligation error rates of PBCV DNA ligase. SBL ligation error rate of PBCV DNA ligase on RNA is variable, depending on the base position. To determine the read-length for direct RNA-templated sequencing, four partially degenerate base-containing k-mers were designed for base 1-4, 5-8, or 9-12, interrogating bases either 5′ or 3′ to the target-specific sequencing primer. For those bases 1 or 2 bases away from the sequencing primer, the single-base discrimination is 94% to 99%; FIG. 8B, at base position 1 and 2, the ligation error rate is ˜2-3% in the forward direction and 3-6% in the reverse direction without any error correction. FIG. 8C, regardless of the ligation error, base calling can be made unambiguously from consensus reads (rSBL NGS) yielding highly accurate sequencing directly from the RNA molecule (as assessed by NGS). After 8 bases in either direction, the sequencing fidelity rapidly drops off to <40%, suggesting a read length of ˜8

FIG. 9: RNA-based SBL using PBCV DNA ligase using four competing oligonucleotides detect up to 50% of the sequencing primer bound to the RNA template within two minutes at 25° C. or 37° C. (bottom left). After 60 min, the sensitivity of RNA SBL is 75% and 90% (bottom left), respectively. The specificity of base recognition is largely invariant of the temperature, salt concentration, or ATP concentration. Without competition, the erroneous ligation rate rises to 25% for this particular RNA template (top left), compared to less than 1% (bottom left) with competition in rSBL.

FIG. 10A-B: FIG. 10A, to label and isolate single cells within tissues, it is critical to have an rSBL-dependent signal amplification method with high SNR with single-molecule sensitivity in situ. Also, it should be practical for most clinical pathology labs for any clinical utility. The sequencing primer incorporates multiple phosphothioate groups, and it prevents the degradation of digoxigenin- or biotin-labeled sequencing probes. Wild type-specific sequencing probes are unlabeled, enabling them to compete and reduce false positives without being detected. Without rSBL ligation, the sequencing probes are degraded and washed off, yield high SNR labeling of specific sequences (non-wild type) in mouse pancreatic cancer cell line (mM1 DLTB). k-mers are labeled with digoxigenin. The sequencing primer (SP) contains three phosphothioate groups at the 3′ terminus, which prevents its degradation by Exonuclease I & III. After SBL on RNA (rSBL) in fixed cells, an anti-digoxigenin antibody coupled to Horse Radish Peroxidase (HRP) is used to label cells containing rSBL products. In the absence of SplintR (or PBCV-1 DNA ligase), un-ligated k-mers are washed away, lowering the background signal by ˜5-to-10-fold. The residual signal is due to a combination of non-specifically bound k-mers or anti-digoxigenin antibodies. FIG. 10B, without phosphorothioate-mediated inhibition of 3′ exonucleases (right), >95% of the probes are degraded even after rSBL, suggesting that off-target rSBL events could largely be eliminated. Here, it is critical to also use RNase H and/or RNase cocktail, since 3′ exonucleases do not work against DNA:RNA hybrids (not shown). This requires immobilizing the rSBL product so that they do not diffuse away with RNases (next step). Non-specifically bound k-mer probes labeled with digoxigenin can be degraded using exonuclease I & III. As these nucleases cannot digest k-mers that remain hybridized to RNAs, RNases is necessary after immobilizing or fixing rSBL products inside the cell.

FIGS. 11A-B: FIG. 11A; to immobilize and amplify the rSBL signal with high specificity in solution, in situ, or in intact cells, a short linker-adapter-like sequences (17-mers) capable of self-assembly by concatamerization is added during rSBL or subsequently. The concatemer formation is rapid, yielding >50-fold signal amplification in vitro, and the increased length of the rSBL product prevents it from diffusing away from the cell. To further amplify chromogenic or fluorescent signal, monomers containing a functional moiety (i.e. biotin, digoxigenin) are polymerized and extended from the k-mer tail after ligation; FIG. 11B, the concatemer formation is initially promiscuous; however, they can be efficiently digested away using 3′ exonucleases. The correct ligation of rSBL adds the phosphothioate group (stars) to the concatemer, blocking the digestion of the signal from true rSBL molecules. Here, the whole reaction including sequencing, signal amplification, and immobilization and read-out is combined into a two to three simple steps. Short monomers (8-bases) are pre-annealed in a tube from two partially overlapping oligonucleotides. Such monomers form concatemers in situ in the presence of DNA ligase. To determine whether such concatemers are linear, Exo I & III are used. The polyacrylamide gel demonstrates that concatemers are linear and that the presence of biotin or digoxigenin (circles) on the DNA does not affect exonuclease digestion. Because the sequencing primer (SP) has 3′ phosphothioate modifications (stars) that protect ligated concatemers from exonuclease-mediated digestion, exonucleases can be added to the mixture after ligation to degrade non-specific k-mers and reduce the level of false positives.

FIG. 12: Using streptavidin-coated beads or fixed cells or tissues, one can detect single cell variants using sequencing primers immobilized on a paper-strip or any other portable substrates. In two to three steps, rSBL-capable strips specific for non-wild-type sequences can be formed, amplified using concatemers, and coupled to enzyme-linked colorimetric assays (e.g. ELISA) in a single tube. This could enable on-site determination of the presence of mutations or cancer cells in <1-hour. This could be used by clinicians, surgeons, or pathologists who need real-time data to determine the size, quality, and extent of local excision or removal of tumors, if needed. If wide dissemination of single tumor cells are already present at the time of surgical treatment, it could alter the actionable course. The previous approach using in situ cell-based rSBL will also enable pathologist to confirm this result the following day by visualizing individual tumor cells in the adjacent tissues. The sequencing primer is modified using acrydite and immobilized onto a paper, glass, or semiconductor strip. A biological specimen containing mutant nucleic acids of interest is added to a reaction chamber or tube containing the sequencing primer-bearing strip as well as k-mer interrogation probes directly or indirectly conjugated to reporter enzymes and DNA ligase. The excess probes or non-specific ligation products are degraded and washed off using error-correcting endonucleases and/or exonucleases. The enzyme-linked strip is incubated with a small molecule substrate to generate a colorimetric readout that is correlated with the amount of functionally relevant mutations in the original specimen. One application of such a kit is for on-site determination of the presence of mutations or cancer cells.

FIG. 13: Instead of one sequencing primer, multiple target-specific sequencing primers can be used to bind to their complementary RNA sequences, followed by washing on beads, cells, or paper strips. The rSBL sequencing probes specific for non-wild type sequences can be designed against ˜300 validated cancer driver genes and their common amino-acid mutations, enabling one to detect rare tumor cells bearing any one or combinations of 300 driver mutations for cell labeling and cost-effective mutation sequencing.

FIG. 14: Mutation detection strategies and applications. From simple and low-cost genotyping assays (e.g. allele-specific PCR) to highly sensitive, comprehensive, and unbiased sequencing methods (i.e. NGS), the trade-off between the cost and the sensitivity of detecting rare genetic variants is a common theme and is one of the main factors in determining their usage in precision medicine or oncology.

FIGS. 15A-B: FIG. 15A, Cells in the human body accumulates hundreds of de novo mutations over the span of a lifetime, especially in frequently dividing cell types. While only a fraction of somatic mutations modifies the protein function, harmful variants eventually emerge and contribute to age-related disorders, including cancer; FIG. 15B, therefore, timely detection of pathogenic protein alterations is an important goal in disease screening, early diagnosis, and residual disease follow-up. An ideal assay should detect clinically or functionally consequential mutations 1) that alter the protein function, 2) that are expressed in diseased tissues, 3) across multiple loci, and 4) that are comprised of multiple sequence alteration types (i.e. missense, nonsense, frame-shift, fusion).

FIGS. 16A-B: FIG. 16A, the most direct way to identify mutated proteins expressed in cells is to label the protein using antibodies specific for a given amino acid alteration; however, steps required to generate highly specific and sensitive antibodies are considerable, and cross-reactivity to other epitopes is common. Here, each amino acid is represented by a codon triplet present in the mRNA; FIG. 16B, instead of relying on tRNAs and the ribosome to translate each codon into an amino acid, partially degenerate missense k-mer probes (mixed base D. A/G/T) can recognize a specific types of amino acid alterations directly. This requires RNA labeling methods capable of discriminating at least three consecutive single nucleotides (i.e. codon) with high sensitivity and single-nucleotide specificity.

FIG. 17: The normal amino acid for KRAS at amino acid position 12 is Glycine (or GTT). A single point mutation can change this codon into Ser, Arg, Cys, Asp, Ala, or Val. The mutant codons have complementary k-mers whose base composition can be represented by 3 oligonucleotides containing mix bases during synthesis (D or B). One of the three k-mers represents synonymous mutations (Glycine or G); therefore, only two probes are required to detect all non-synonymous codon alterations at KRAS G12 or any other amino acids. In competitive probe ligation, all sequences that represent Glycine are included in the reaction; however, these wild-type probes are blocked from signal amplification (e.g. no amplification adapter sequence).

FIG. 18-B: FIG. 18A, functional mutations in many tumor suppressors (i.e. 7P53) lead to premature stop codons or protein truncation events. While such events are common across tumor suppressors, antibodies capable of detecting protein truncations do not exist. By using k-mers that incorporate the three stop codon sequences, it is possible to recognize mutational events that lead to early termination of protein translation; FIG. 18B, small insertions or deletions are also common in cancer. Since in-frame deletions differ by multiples of 3 bases, their sequences can be predicted. One can then generate a pool of k-mers representing shifted sequences resulting from a given deletion.

FIGS. 19A-B: FIG. 19A, KRAS mutations are largely comprised of non-synonymous mutations at G12, whereas TP53 mutations are predominantly premature stop codons. Top ten codon mutations for KRAS and TP53 are shown. These codon changes are present in 22% of all sequenced tumors (88% of all KRAS mutations and 26% of all TP53 mutations); FIG. 7B, 50 missense or nonsense codon mutations in KRAS or TP53 are found in 50% of all sequenced human tumors (MSKCC IMPACT pan-cancer clinical sequencing study, Nature Medicine 2017). This translates into a pool of only ˜160 k-mer oligonucleotides, which is still less than traditional SBL interrogation probes for NGS with 6-degenerate bases (e.g. NNNNNN, or 4096 competing oligonucleotides).

FIG. 20: DNA ligases are slow to ligate nicked DNA strands, if it contains a base-pair mismatch. This dramatically improves the specificity of allele discrimination. The combination of competitive ligation, rapid equilibration of competing k-mer probes, and large difference between correct (SP-PM) and mismatched (SP-MM) probe ligation is key to sequencing-by-ligation. If these parameters are met, enzymatic or chemical ligation methods are capable of sequencing-by-ligation (SBL).

FIG. 21: To characterize the parameter necessary to perform SBL on the RNA template using PBCV-1 DNA ligase (SplintR, NEB), the RNA target-specific sequencing primer is immobilized on beads, glass, or fixed cells. After hybridization-based capture of RNA targets, the excess material is washed away. Partially degenerate k-mers with Tₘ of 37° C. (˜9-12 bases) are added in conjunction with SplintR to the RNA-DNA sequencing primer hybrid at 37° C. for 60 minutes. The excess k-mers are washed away, followed by PCR or qPCR of the fully ligated SBL product. The PCR fragments are analyzed using capillary electrophoresis, Sanger, or NGS, depending on the number of unique RNA targets interrogated.

FIGS. 22A-B: FIG. 22A, when individual k-mers are used for SplintR-mediated ligation on RNA in the absence of competition, the erroneous ligation product is found in up to 25% of all interrogated RNAs. If the k-mer is perfectly complementary (SP-PM), up to 75% of all RNAs generate correctly ligated DNA fragments within 5 minutes; FIG. 22B, when partially degenerate k-mers are used for ligation in competition, the erroneous ligation rate drops from 25% to 1%. If one lowers the reaction temperate below Tₘ of k-mers, the ligation efficiency is reduced from 95% to 70%. The enzyme activity is similar between 25° C., and 37° C.; however, the ligation-incapable mismatch k-mers cannot be exchanged for matched k-mers at Tᵣₓₙ<Tₘ.

FIGS. 23A-B: FIG. 23A, a major parameter that determines the relative amount of RNA templates interrogated by SBL is the identity of 5′ phosphorylated base. If the 5′ phosphorylated base is either A or T, the SBL efficiency is 95%; however, C or G yields <50% ligation regardless of adjacent sequences or changes in reaction conditions; FIG. 23B, the failure of SBL is due to the accumulation of 5′ adenylated sequencing primer, suggesting that SplintR is unable to complete the last step in DNA ligation efficiently on 5′ adenylated C or G.

FIG. 24: the SBL error rate of SplintR on RNA is variable, depending on the base position. The read-length of SplintR-based SBL is similar to that of T4 DNA ligase-based SBL used in NGS (i.e. ABI SOLiD, Complete Genomics).

FIG. 25: erroneous SBL creates DNA:RNA mismatches that are recognized by T7 Endonuclease I.

FIG. 26: Following RNA-templated SBL (rSBL) using k-mers that define any number of sequence categories (i.e. missense mutations), PCR handles attached to the SBL product can be used for real-type quantitative PCR (RT-qPCR), TaqMan PCR, digital droplet PCR, or in situ PCR. This enables one to rapidly quantify the amount of potentially deleterious RNA within the sample and screen a large number of samples, followed by Sanger sequencing or NGS of those specimens that contain deleterious mutations.

FIG. 27: k-mer SBL on mRNA followed by qPCR. The codon-specific sequencing primer is immobilized on Dynabeads. After hybridization-based capture of mRNAs, the excess material is washed away. Partially degenerate k-mers with Tₘ of 37° C. are added in conjunction with SplintR at 37° C. for 60 minutes. Wild-type and synonymous sequences are recognized by respective k-mers incapable of signal amplification (i.e. no PCR handle). The excess k-mers are washed away, followed by PCR or qPCR of the fully ligated SBL product. The PCR fragments are analyzed using SYBR-Green qPCR, TaqMan PCR, or digital droplet PCR.

FIGS. 28A-B: FIG. 28A, the relative Ct value of KRAS G12 and Q61 codon SYBR-Green qPCR using non-synonymous k-mer SBL on purified RNA mixtures (wild-type vs. mutant %). The ΔCt value is normalized against samples that do not contain mutant RNAs. The slope of ΔCt vs. missense RNA % is ˜1.1 (R²=0.7-0.9), showing that non-synonymous k-mer SBL is linearly quantitative; FIG. 28B, the relative Ct value of KRAS G12D, G/2R, and G/2V specific k-mer SBL on purified RNA mixtures. SYBR-Green qPCR has a limited detection sensitivity due to non-specific amplification (i.e. primer dimers), and the SBL error rate (1-5%) prevents k-mer SBL on RNA from detecting VAF <5%; however, the method discriminates mutant samples with unknown with VAF >10°,% with ΔCt>3 directly from RNA.

FIG. 29: The relative Ct value of KRAS G12 codon SYBR-Green qPCR using non-synonymous k-mer SBL on purified RNA mixtures (wild-type vs. mutant %). The ΔCt value is normalized against samples that do not contain mutant RNAs. The correct RNA capture probe or sequencing primer (‘Correct SP’) is 100% complementary to the KRAS G12 RNA target, while the incorrect RNA capture probe (‘Partial SP’) contains 7-10 mismatched bases, indicating that multiple target-specific sequencing primers can be used simultaneously.

FIG. 30: Non-synonymous k-mers for KRAS G12 and Q61 are used for PCR, followed by agarose gel electrophoresis. Due to the ligation error rate (1-5%), PCR products can be observed at 0% VAF (false positive) at PCR cycle of >20. Another source of false positives are PCR contaminations. Such factors limit the sensitivity and specificity of qPCR. The asterisk marks indicate primer dimers that accumulate around Ct 20, which is another factor limiting SYBR-Green qPCR.

FIG. 31: Instead of sequencing all samples, only those samples expressing mutant or deleterious mRNAs are sent for Sanger sequencing. In this example, completely degenerate k-mers SBL on RNAs are used. Without the use of partially degenerate k-mers (missense-specific), the Sanger sequencing specificity is solely a function of VAF (>50%; top left panel) as the wild-type sequence (‘GGC’) overwhelms the signal at lower VAFs (<50%).

FIG. 32: DNA or RNA-templated SBL using k-mers that define any number of sequence categories (i.e. missense mutations) can be used to form or activate sequencing primers for polymerase (Sequencing-By-Synthesis, or SBS) or ligase (SBL) extension cycles.

FIGS. 33A-B. FIG. 33A, partially degenerate k-mers are first ligated to the anchor sequencing primer on the DNA template to activate NGS sequencing primers only from templates containing functional sequence variants. Different sets of k-mers are labeled with FAM, Cy3, TexRed, or Cy5, as indicated by stars. After missense or nonsense k-mers have been ligated to the anchor primer, excess probes are washed off, and the end of each ligation product is cleaved, releasing the terminator dye and enabling another round of ligation or polymerase-based single nucleotide extension (i.e. Illumina SBS). The k-mer set that represents wild-type or functionally silent mutations do not contain the terminator cleavage site, and they cannot be extended for SBS; FIG. 33B, partially degenerate k-mers accurately prime DNA amplicons in situ based on the composition of single nucleotide bases. One can assess the accuracy by quantifying the frequency of fluorophore co-localization. Here, partially degenerate k-mers containing mixed bases (K or R) overlap if they interrogate the same base, while they glow in one or no color if each k-mer recognizes unique base (non-overlapping nucleotides, such as A or T, between mixed bases, such as K or R).

FIGS. 34A-B: FIG. 34A, once circulating tumor DNA (ctDNA) is isolated from the blood, its ends are blunt-ended and 5′ phosphorylated. Exonuclease III generates ssDNA of ˜70 bases, which is circularized using CircLigase. Prior to exonuclease digestion, adaptors containing unique molecular identifiers (UMIs) or RCA priming site could be ligated to double-stranded ctDNA fragments. Biotinylated RCA primers with universal adapter- or target-specific sequences are used to amplify circularized ctDNA. Single-molecule ctDNA amplicons are then immobilized on streptavidin-coated glass flow cell for k-mer SBL, cleavage, and SBL/SBS; FIG. 34B, this enables one to observe fluorescence associated with a sequencing reaction only from those amplicons that contain missense or nonsense mutations, eliminating the need to sequence ultra-deep (counting millions of wild-type sequences) to detect rare variants in NGS.

FIG. 35A-C: FIG. 35A, High-throughput sequencers (ABI, Illumina, PacBio, Oxford) interrogate all nucleic acid molecules as long as they possess suitable adapter sequences and can be separated in space (i.e. arrays, flow cells). FIG. 35B, For example, optical imaging-based sequencers (i.e. Illumina HiSeq) generate immobilized PCR amplicons within the flow cell in situ, which are then interrogated using SBS and fluorescence imaging. While higher cluster densities enable more reads per lane, over-crowding limits the accuracy of base calling due to the limited optical resolution. To generate millions to billions of reads, one must scan a large area across multiple sequence lanes. FIG. 35C, Using k-mer SBL, one can now generate optical fluorescence from only those molecules containing deleterious mutations in a multiplexed manner. Because ctDNA molecules are rare, over-crowding is not an issue, and ultra-compact imaging devices can now be used to count ctDNA amplicons rapidly and cheaply. This enables ultra-sensitive ctDNA counting at low-cost for frequent or repeated ctDNA monitoring, improving its diagnostic specificity and sensitivity, without having to resort to traditional ultra-deep NGS.

FIG. 36: Functional mutation-specific k-mer SBL on RNA (rSBL) can be used to label single-cells in situ without the need for mutation-specific antibodies, enabling one to visualize and quantify rare, resistant, disseminated, or residual cancer cells in the patient tissue, as well as many other applications. Currently, the main way to identify cancer cells is to use general tissue dyes (i.e. H&E) or cancer biomarker antibodies or FISH probes; however, these approaches are not suitable for rare cancer cells that do not have biomarkers.

FIGS. 37A-B: FIG. 37A, 30-nt sequencing primer specific to human IDH1 possesses an adapter sequence (orange). 12-nt k-mers (three non-synonymous point mutation oligonucleotides) also possess an adapter sequence (orange). After rSBL, CircLigase joins the 5′ and 3′ ends of the adapter sequence to form a circular product in a template-dependent manner. Subsequently, rolling circle amplification (RCA) is used to visualize rSBL products in situ; FIG. 37B, human astrocytes containing IDH1 R132H (CGT>CAT) are recognized by ATG anti-sense probes within ADG anti-sense oligonucleotides. Despite 10-times molar excess of k-mers compared to the sequencing primer, mutation-specific amplicons are formed only in IDH1 R132H cell lines but not in wild type cell lines.

FIGS. 38A-B: FIG. 38A, k-mer SBL products hybridized to the mRNA in fixed cells can also be amplified into fluorescently labeled amplicons using previously published methods (e.g. SNAIL from Wang et al., (2018)). A third ssDNA oligonucleotide serves as a splint to circularize the SBL product using T4 DNA ligase. Once target specific k-mers are circularized, they can be amplified using Phi29 DNA polymerase (RCA). AP1 and AP2 indicate adapter sequences included in the SBL product; FIG. 38B, another approach is to hybridize a long concatemer of fluorescently labeled ssDNA. Such extensions could be made to branch multiple times for arbitrarily high signal-to-noise ratio (SNR); however, removing excess or un-ligated k-mers becomes critical to reduce false positives.

FIGS. 39A-B: FIG. 39A, previously described methods (i.e. SNAIL) require additional probe hybridization steps, probes, or incubation cycles to generate sufficiently high SNR. Alternatively, k-mer SBL products can be self-circularized using CircLigase, followed by RCA using a universal RCA primer; FIG. 39B, RCA can be performed in the presence of random hexamers or multiple directional RCA primers for hyper-geometric DNA amplification (multiple displacement amplification, or MDA) in situ. While the resulting product yields dsDNA, a significant fraction remains as ssDNA, enabling one to perform in situ hybridization directly for fluorescence microscopy.

FIG. 40: The k-mer based probe ligation on DNA or RNA templates can be linked to enzyme-based immunosorbent assay (ELISA)-like platform to detect or quantify the level of functionally relevant mutant molecules present in the tissue lysate or other biological fluids.

FIGS. 41A-B: FIG. 41A, KRAS G12D mutation bearing RNA templates are immobilized on streptavidin-coated beads. The sequencing primer is pre-hybridized to the RNA template, followed by a wash cycle. Subsequently, a non-synonymous mutation-detecting k-mer probe along with a wild-type competitor probe is added to the reaction tube for DNA ligation on RNA. The non-synonymous k-mer probe is modified at the 5′ end with digoxigenin, enabling it to be detected using an anti-digoxigenin antibody conjugated to alkaline phosphatase (AP); FIG. 41B, in the first iteration of AP-PNPP-based colorimetric detection of functionally relevant nucleic acids, 2-pg RNA generated a visible colorimetric read-out after 6 hours. With additional amplification of the k-mer associated handle (FIG. 11), the speed and sensitivity can be improved significantly.

FIG. 42: Functionally relevant mutations include Cas9-induced indels used for cell line- or animal-based pooled screening to identify gene targets or critical amino acids. Programmable k-mers for rSBL can be used to discriminate types of Cas9-indels in situ or in vivo to identify genes or amino acids critical to their function in vivo. This enables a large number of genes or amino acids to be functionally screened (i.e. gene knockout) in their native tissue environment, which differs from traditional pooled screening in vitro.

FIG. 43: Cas9-induced indels are located 2-3 bases away from the PAM site. In addition, a large fraction of mutations is comprised of small deletions (1-6 bases). This enables one to design k-mers for rSBL capable of recognizing in-frame or out-of-frame mutations caused by Cas9.

FIGS. 44A-B: FIG. 44A, if the translated protein has an out-of-frame mutation near the amino terminal end of the protein, it generally leads to complete loss-of-function; however, in-frame mutations lead to a loss-of-function phenotype only if the deleted amino acid residue is critical for the protein function. Such amino acids or regions could be targets for therapy; FIG. 44B, current methods rely on cell culture systems to perform amino acid or domain mapping studies to identify druggable proteins. However, native tissue environment significantly alters cell signaling and phenotype for many cell types, including cancer cells. Therefore, methods that can generate Cas9-induced indels in vivo (via viral delivery), followed by single-cell in situ detection of in-frame indels, could enable a broad range of drug target discovery that are directly relevant to in vivo physiology.

FIGS. 45A-B: FIG. 45A, somatic non-synonymous mutations are functional because they alter the protein function; however, other types of short sequence variants are also functional if they promote aberrant protein homeostasis (e.g. degradation, solubility); FIG. 45B, larger non-coding triplet variants (e.g. nucleotide repeat expansion) are implicated in neurodegenerative disorders. rSBL enables one to interrogate triplet nucleotide expansions using multiple cycles of ligation-based sequencing primer extension.

FIGS. 46A-B: FIG. 46A, the read-length in SBL depends on the ‘footprint’ of DNA ligase and the number of re-ligation cycles after cleavage of reversible terminators. To cleave the reversible terminator from the SBL product hybridized to RNA, inosine-specific Endonuclease V is used. Because multiple cleavage sites exist, phosphothioate modification are used to direct cleavage exactly 2-bases away from inosine. The position of inosine can vary depending on the size of repetitive units interrogated; FIG. 46B, SBL is performed in situ within tissue sections containing molecular DNA amplicons. The sequencing primer bears FAM at the 3′ end, while SBL interrogation probes containing inosine are conjugated to Cy3 at the 5′ end (terminator of ligation). SBL products (FAM+Cy3) are cleaved using Endonuclease V at the 5′ end but not at the 3′ end. Endonuclease V cleavage is >95% complete after 10 minutes, removing the previous fluorophore and exposing 5′ phosphate for another round of SBL for primer extension. Endonuclease V does not lead to degradation of RNA when used for RNA-templated SBL (rSBL).

FIGS. 47A-B: FIG. 47A, in order to detect the size of repeat expansion in single cells or in situ, RNA-templated SBL (rSBL) using repeat-specific k-mers are sequentially added using cyclic ligation and cleavage; FIG. 47B, as long as additional repeats are present, each sequencing round generates rSBL products with a fluorophore molecule; however, ligation is not possible at the end of the repeat expansion, which can be identified by previously fluorescent signal that is lost. The number of re-ligation cycles need to reach the end of repeat expansion corresponds to the size of repeat expansion, which can be performed in situ to detect cells predisposed to develop neurodegenerative disease. The programmability of k-mers enables one to interrogate repeats of complex composition and discriminate closely related triplet expansions in the genome.

FIGS. 48A-C: FIG. 48A, Programmable k-mer for rSBL containing partially degenerate bases (e.g. mixed base presentation S or B in the probe sequence attached to FAM or Cy3) can be used to group genes that are detected based on shared sequence motifs. Given the single-base specificity of rSBL, multiple probes that individually interrogate orthogonal sequence motifs can be used to group Genes 1-3 and Genes 4-6. Each group-specific k-mer is represented by one oligonucleotide; FIG. 48B, by performing sequential rSBL across major functional classes of expressed mRNAs or non-coding RNAs, in which each cycle is followed by inactivation or cleavage of fluorophores (e.g. Endonuclease V, photobleaching), signaling pathways and gene ontology can be ‘stained’ and visualized using fluorescence microscopy after suitable signal amplification of ligated probes. Here, each fluorophore represents different cell or gene expression state for a given gene ontology category; FIG. 48C, instead of enumerating individual RNA molecules and their identity in situ, programmable pathway-specific rSBL using k-mers enables one to reconstruct functional signaling pathways using a small number of interrogation probes and imaging cycles using low-magnification microscopy.

FIG. 49: Anti-sense oligonucleotides are suitable for RNA-based therapeutics, if their stability and delivery efficiency issues can be optimized. The single-base specificity of k-mer based rSBL is a function of primer design, complexity, and thermodynamics, in addition to DNA ligation kinetics. Therefore, rSBL in living cells can occur if the rate of DNA ligation can be tuned, and this property can be made to trigger cytotoxicity to eliminate cells bearing deleterious somatic mutations.

FIGS. 50A-B: FIG. 50A, Anti-sense oligonucleotides representing sequencing primers and k-mers are delivered to live cells or tissues via local infusion, electroporation, or liposomes. The target-specific sequencing primer and k-mers are modified at 5′ and 3′ ends for copper-free alkenyl/azide click chemistry or ribozymes for intracellular DNA ligation. rSBL products are capped at both ends and resist endogenous exonuclease digestion, FIG. 50B, the proximity of the two capping groups enables one to conjugate them to nanoparticles that convert and amplify external energy (e.g. electric current, radiation, light) for rSBL-conditional cell cytotoxicity.

FIG. 51: Additional applications include labeling and sorting of rare circulating tumor cells for genome sequencing or proteome analysis. Here, cancer cell-specific antibodies are no longer absolutely required, since the presence of truncal mutation (e.g. KRAS) identifies cells as cancerous by definition. k-mer based rSBL probes can also be conjugated to metal isotopes for multiplexed cartography of somatic mutations in clinical or FFPE tissue sections using imaging mass cytometry.

FIG. 52: Current in situ nucleic acid ligation methods (from left to right, LISH, Ligation in situ Hybridization as disclosed in, e.g., Credle et al. (2017); iLock as disclosed in e.g. Krzywkowski, T., et al. (2017) and Krzywkowski, T., et al. (2019); and Sequencing by Ligation as disclosed in, e.g., Lee, J. H., et al. (2015) and how they compare to ProRSBL disclosed herein (far fight). Note that iLock and chimeric probes are improvements made to PLP to increase specificity. ProRSBL uses probes that are programmed (orange nucleotides) for selective RTDL and include cleavage sites (I) for additional rounds of ligation.

FIG. 53: Outline of ProRSBL: The target RNA and a 5′ phosphorylated sequencing primer (˜30-40 mer) are hybridized and immobilized. Probes ending in degenerate bases are used to interrogate a base of interest in competition. The melting temperature of the probes must be less than the reaction temperature. Following ligation, RNase treatment removes the template RNA and the ligation product is analyzed by the method of choice, including in silt sequencing (ISS).

FIGS. 54A-B: FIG. 54A, Competition between perfectly matched (PM) and mismatched (MM) probes reduces erroneous ligation to a sequencing primer (SP) compared to reactions where only mismatched probes are present; FIG. 54B, Ligation involving only one perfectly matched probe reaches 50% of the maximum product quantity (area under curve using capillary electrophoresis) within 5 min whereas competitive ligation between probes ending in NNNN (256) probes reaches 50% of maximum product quantity within 20 minutes.

FIG. 55: Ligation efficiency plotted against 5′,5′-Adenylyl pyrophosphoryl DNA (AppDNA) concentration with the sequencing primer 5′ end identified. The reduction in ligation efficiency for sequencing primers beginning with C or G (5′ end) is due to the accumulation of adenylated products unable to complete ligation. For sequencing primers beginning with A or T, no such limitations are observed.

FIG. 56: Determining the read length of forward and reverse ProRSBL. Probes with degenerate quartets (NNNN) scanning positions 1-4, 5-8, and 9-12 upstream (5′ phos) and downstream (3′ OH) of the sequencing primer were used for ProRSBL, and ligation products were analyzed using NGS (MiSeq). Four degenerate bases were interrogated simultaneously to reduce the probe library complexity.

FIGS. 57A-C: FIG. 57A, A schematic depicting endonuclease V cleavage upstream of inosine, which results in a 5′ phosphorylated donor suitable for ProRSBL. A phosphorothioate bond (represented by a dot) restricts enzymatic cleavage to the second phosphodiester bond downstream of inosine; FIG. 57B-FIG. 57C, Cleavage kinetics of an inosine bearing RNA:DNA duplex, followed by ProRSBL. The majority >95% of the starting cleaved substrate is either ligated or present as adenylated DNA. P-values calculated using Welch Two Sample t-test. **<0.005. Error bars represent standard error of mean.

FIG. 58: An example of ProRSBL probe design to agnostically enrich for KRAS codon 12 variants (without specifying the exact sequence). NOT logic gates exclude wildtype sequences, followed by AND gates to assemble codons resulting in synonymous, mis-sense and non-sense mutations. The entire mutation rage, or a subset, can be detected using ProRSBL. In the depicted example, only single-nucleotide variants (sense and missense highlighted in orange) were pursued.

FIG. 59: Schematic for testing ProRSBL against KRAS codon 12. Probes were designed to amplify ligation products at single-nucleotide variant but not wildtype codon 12. Mutation detection probes having partially degenerate bases (orange nucleotides) and an amplification arm at the 5′ end. Probes detecting the wildtype codon can ligate but are not amplified due to lack of proper primer site.

FIGS. 60A-B: FIG. 60A, Serial dilution of mutant KRAS synthetic RNA templates (100%, 10%, 1% and 0%) followed by ProRSBL and qPCR NGS. FIG. 60B, NGS of ligation products following ProRSBL on synthetic RNA templates to detect specific KRAS mutations in codon 12 at different concentrations relative to other probed variants.

FIG. 61: Schematic for testing ProRSBL in situ against IDH1 codon 132. The sequencing primer and probes were designed to allow for circularization using CircLigase II followed by RCA. During ProRSBL against mutant IDH1 codon 132, ligation products occurring at the wildtype codon are not circularized due to presence of a 5′ inverted T (Ref) instead of a 5′ phosphate.

FIG. 62: RCPs (small, bright dot-like circles) from in situ ProRSBL against mutant IDH1 codon 132 (i.e. not GCA and DAPI (larger, amorphous circles) Scalebar=20 microns.

FIG. 63: Quantification of RCPs derived from mutant specific probes in cells overexpressing wildtype or mutant IDH1. ***<0.0005. Error bars represent standard error of mean.

FIG. 64: ProRSBL is a framework for integrated multiple statements about cellular RNA content for advanced profiling. In the example provided, the expression of Gene X, but not Gene Y in the present of missense or nonsense mutations in a specific codon encoding glycine in Gene Z are assessed using ProRSBL. The three statements could be combined into a new molecule via in situ PCR stitching or concatemer forming primer exchange reaction cascades.

FIG. 65: Minimal effect of Endonuclease V on total RNA. An electronic gel image produced by Agilent Bioanalyzer using RNA Nano chip after time course for Endonuclease V digestion with human total RNA (50 ng/μL).

### DETAILED DESCRIPTION OF THE INVENTION

The present invention provides a method for determining the presence or absence of variant ribonucleic acid molecules in a population of ribonucleic acid molecules, wherein the reference sequence of the variant ribonucleic acid molecules is known, the method comprising:
(a) interrogating the population of ribonucleic acid molecules with:
   (i) a plurality of primer molecules comprising nucleotides, wherein:
      (1) at least 8 consecutive nucleotides, starting at the 5′ or the 3′ end of each primer molecule, are fully complimentary to consecutive nucleotides of the reference sequence, and
      (2) the primer molecules have a melting temperature of at least 50° C. when hybridized to their complimentary ribonucleic acid molecules in the population of ribonucleic acid molecules; and
   (ii) a plurality of probes comprising L S nucleotides, wherein:
      (1) nucleotides L are complimentary to: (1) the reference sequence that is adjacent to the nucleotides of the reference sequence that each primer molecule is fully complimentary to, or (2) a sequence that differs from (1) at one or more nucleotide bases along the length of L.
      (2) nucleotides S are fully complementary to the reference sequence, and
      (3) L+S is 8 to 12 and L is at least 1,
   so as to saturate the population of ribonucleic acid molecules with the probes and primer molecules such that the probes and primer molecules are adjacent to one another when hybridized to their respective complimentary sequences on the ribonucleic acid molecules, wherein if the 5′ end of the primer molecules are adjacent to the 3′ end of the probes when both are hybridized to their respective complimentary sequences then at least 8 consecutive nucleotides at the 5′ end of the primer molecules are fully complimentary to nucleotides of the reference sequence and the primer molecules have a 5′ phosphorylated A or T, and wherein if the 5′ end of the probes are adjacent to the 3′ end of the primer molecules when both are hybridized to their respective complimentary sequences then at least 8 consecutive nucleotides at the 3′ end of the primer molecules are fully complimentary to nucleotides of the reference sequence and the probes have a 5′ phosphorylated A or T:
(b) ligating the probes to their respective adjacent primer molecules so as to form ligated nucleic acid molecules, wherein the probes are ligated in competition under conditions favoring the ligation of fully hybridized probes over partially hybridized probes, wherein such conditions comprise using a reaction temperature that is about the melting temperature of a probe of length L+S that is fully hybridized;
(c) detecting the presence of ligated nucleic acid molecules that are complementary to a sequence that differs from the reference sequence;
thereby determining the presence or absence of one or more variant ribonucleic acid molecules in the population of ribonucleic acid molecules.

In an embodiment, the primer molecules and probes form the following sequence, read 3′ to 5′, when hybridized to their respective complimentary sequence on a ribonucleic acid molecule in the population of ribonucleic acid molecules, wherein the numbers in brackets represent the number of nucleotides, N represents nucleotides of the primer molecule that are fully complimentary to the reference sequence, P represents additional nucleotides of the primer molecule, and X is any whole number sufficient for the primer molecules to have a melting temperature of at least 50° C. when hybridized to their complimentary ribonucleic acid molecules in the population of ribonucleic acid molecules:
(a) P_{(X)}N₍₈₊₎L₍₁₋₈₎S₍₀₋₁₁₎, wherein the 5′ nucleotide of N is a phosphorylated A or T, or
(b) S₍₀₋₁₁₎L₍₁₋₈₎N₍₈₊₎P_{(X)}, wherein the 5′ nucleotide of L is a phosphorylated A or T
wherein the ligation in step (b) occurs between L and N.

In an embodiment, the method further comprises a step of removing excess unhybridized or partially hybridized primer molecules and/or probes after step (a).

In an embodiment, step (c) comprises sequencing the ligated nucleic acid molecules.

In an embodiment, L is 1, 2, 3, 4, 5, 6, 7, or 8. In an embodiment, L is 3.

In an embodiment, the plurality of probes consists of probes complimentary to each respective single base variant along the length of L.

In an embodiment, the plurality of probes consists of probes complimentary to each possible single base variant along the length of L other than non-actionable sequences, synonymous mutations, non-functional polymorphisms, or mutational patterns not observed in the human population.

In an embodiment, some or all of the plurality of probes comprise a fluorophore.

In an embodiment, some or all of the plurality of probes further a signal amplification functional group.

In an embodiment the signal amplification functional group is horseradish peroxidase, alkaline phosphatase, digoxigenin, or fluorescein isothiocyanate (FITC).

In an embodiment, some or all of the plurality of probes comprise an amplification sequence. In an embodiment, the amplification sequence is an adapter sequence for circularization and rolling circle amplification (RCA). In an embodiment, the amplification sequence is a sequence for hybridization of a PCR primer.

In an embodiment, some or all of the plurality of probes comprise a barcode. In an embodiment, some or all of the plurality of probes comprise a cleavable terminator.

In an embodiment, the preferably cleavable terminator is an inosine base. In an embodiment, some or all of the plurality of probes comprise a functional group that accepts and transfers energy from an external source for generation of cytotoxic processes.

In an embodiment, the method comprises one or more further rounds of interrogation and ligation, wherein the ligated nucleic acid molecules formed in step (b) serve as the primer molecules for the next round of interrogation and ligation with a plurality of probes designed as described in step (a) based on the nucleotides of the reference sequence that are adjacent to the nucleotides of the reference sequence that such ligated nucleic acid molecule are complementary to.

In this embodiment, some or all of the plurality of probes further comprise a cleavable terminator and wherein the cleavable terminator is cleaved to form a cleaved ligated nucleic acid molecules which serve as the primer molecules for the next round of interrogation and ligation with a plurality of probes designed as in step (a) based on the nucleotides reference sequence that are adjacent to the nucleotides of the reference sequence that each cleaved ligated nucleic acid molecule is complementary to.

In this embodiment, Endonuclease V is used to cleave the cleavable terminator of the ligated nucleic acid molecules.

In an embodiment, probes that are fully complimentary to the reference sequence of the variant ribonucleic acid molecules do not further comprise a signal amplification functional group. In this embodiment, probes that are fully complimentary to the reference sequence of the variant ribonucleic acid molecules do not further comprise an amplification sequence. In an embodiment, probes that are fully complimentary to the reference sequence of the variant ribonucleic acid molecules are tagged with a non-functional control sequence, which control sequence blocks amplification of the ligated nucleic acid molecule. In an embodiment, probes that are fully complimentary to the reference sequence of the variant ribonucleic acid molecules comprise an inverted dT to prevent circularization and rolling circle amplification.

In an embodiment, probes that are fully complimentary to non-actionable sequences, synonymous mutations, non-functional polymorphisms, and/or mutational pattern not observed in the human population do not further comprise a signal amplification functional group. In an embodiment, probes that are fully complimentary to non-actionable sequences, synonymous mutations, non-functional polymorphisms, and/or mutational pattern not observed in the human population are tagged with a non-functional control sequence, which control sequence blocks amplification of the ligated nucleic acid molecule. In an embodiment, probes that are fully complimentary to non-actionable sequences, synonymous mutations, non-functional polymorphisms, and/or mutational pattern not observed in the human population comprise an inverted dT to prevent circularization and rolling circle amplification.

In an embodiment at least 8 consecutive nucleotides starting at the 5′ end of each primer molecule, are fully complimentary to consecutive nucleotides of the reference sequence. In an embodiment at least 9 consecutive nucleotides starting at the 5′ end of each primer molecule, are fully complimentary to consecutive nucleotides of the reference sequence. In an embodiment at least 10 consecutive nucleotides starting at the 5′ end of each primer molecule, are fully complimentary to consecutive nucleotides of the reference sequence. In an embodiment at least 11 consecutive nucleotides starting at the 5′ end of each primer molecule, are fully complimentary to consecutive nucleotides of the reference sequence. In an embodiment at least 12 consecutive nucleotides starting at the 5′ end of each primer molecule, are fully complimentary to consecutive nucleotides of the reference sequence. In an embodiment at least 13 consecutive nucleotides starting at the 5′ end of each primer molecule, are fully complimentary to consecutive nucleotides of the reference sequence. In an embodiment at least 14 consecutive nucleotides starting at the 5′ end of each primer molecule, are fully complimentary to consecutive nucleotides of the reference sequence. In an embodiment at least 15 consecutive nucleotides starting at the 5′ end of each primer molecule, are fully complimentary to consecutive nucleotides of the reference sequence.

In an embodiment at least 8 consecutive nucleotides starting at the 3′ end of each primer molecule, are fully complimentary to consecutive nucleotides of the reference sequence. In an embodiment at least 9 consecutive nucleotides starting at the 3′ end of each primer molecule, are fully complimentary to consecutive nucleotides of the reference sequence. In an embodiment at least 10 consecutive nucleotides starting at the 3′ end of each primer molecule, are fully complimentary to consecutive nucleotides of the reference sequence. In an embodiment at least 11 consecutive nucleotides starting at the 3′ end of each primer molecule, are fully complimentary to consecutive nucleotides of the reference sequence. In an embodiment at least 12 consecutive nucleotides starting at the 3′ end of each primer molecule, are fully complimentary to consecutive nucleotides of the reference sequence. In an embodiment at least 13 consecutive nucleotides starting at the 3′ end of each primer molecule, are fully complimentary to consecutive nucleotides of the reference sequence. In an embodiment at least 14 consecutive nucleotides starting at the 3′ end of each primer molecule, are fully complimentary to consecutive nucleotides of the reference sequence. In an embodiment at least 15 consecutive nucleotides starting at the 3′ end of each primer molecule, are fully complimentary to consecutive nucleotides of the reference sequence.

In an embodiment, the primer molecules comprise 20-50 nucleotides. In an embodiment, 20-50 nucleotides that are complementary to the reference sequence of the ribonucleic nucleic acid molecules.

In an embodiment, each primer molecule comprises an amplification sequence. In an embodiment the amplification sequence is an adapter sequence for circularization and rolling circle amplification (RCA). In an embodiment the amplification sequence is a sequence for hybridization of a PCR primer.

In an embodiment, each primer molecule comprises a signal amplification functional group. In an embodiment the signal amplification functional group is horseradish peroxidase. In an embodiment the signal amplification functional group is alkaline phosphatase. In an embodiment the signal amplification functional group is digoxigenin. In an embodiment the signal amplification functional group is fluorescein isothiocyanate (FITC)

In an embodiment, each primer molecule comprises a blocking group to make the ligated nucleic acid molecules resistant to degradation. In an embodiment, the blocking group is an inverted dT. In an embodiment, the blocking group is a phosphorothioate nucleotide or phosphorothioate nucleotides. In an embodiment, the blocking group is inert spacer moiety. In an embodiment, the blocking group is a locked nucleic acid or locked nucleic acids. In an embodiment, the blocking group is a modified base or modified bases.

In an embodiment, each primer molecule comprises a fluorescent or colorimetric sequence. In an embodiment, each primer molecule comprises an inverted dT. In an embodiment, each primer molecule comprises a phosphorothioate nucleotide or phosphorothioate nucleotides. In an embodiment, each primer molecule comprises a locked nucleic acid or locked nucleic acids. In an embodiment, each primer molecule comprises a modified base or modified bases. In an embodiment, each primer molecule comprises a functional group that accepts and transfers energy from an external source for generation of cytotoxic processes. Accordingly, the invention provides a method of generating cytotoxicity depending on the presence or absence of a variant ribonucleic acid molecule.

In an embodiment, the method further comprises a step of degrading un-ligated, excess, and/or off-target probes after step (b).

In an embodiment, degrading is by an endonuclease. In an embodiment, degrading is by an exonuclease. In an embodiment, degrading is by a surveyor enzyme. In an embodiment, degrading is by a resolvase. In an embodiment, degrading is by a ssDNA-binding protein.

In an embodiment, the exonuclease is Exonuclease I. In an embodiment, the exonuclease is T7 exonuclease. In an embodiment, the exonuclease is Exonuclease III.

In an embodiment, the endonuclease is T7 endonuclease I.

In an embodiment, the exonuclease is used in combination with RNase H and/or an RNase cocktail;

In an embodiment, degrading comprises the use of exonucleases that remove bound RNA to degrade partially hybridized probes;

In an embodiment, degrading of bound RNA results in the diffusion of the ligated product for in situ applications in fixed cells or tissues;

In an embodiment, degrading further comprises hybridization independent degrading.

In an embodiment, degradation of ligated nucleic acid molecules is blocked by an inverted dT, phosphorothioate nucleotide, or inert spacer moiety from the primer molecule.

In an embodiment, partially hybridized probes of step (b) are in a complex with DNA or RNA molecules or are non-covalently associated with proteins or other cellular material.

In an embodiment, the method further comprises a step of amplifying the ligated nucleic acid molecules before step (c).

In an embodiment, the step of amplifying the ligated nucleic acid molecules comprises multiple displacement amplification (MDA).

In an embodiment, the step of amplifying the ligated nucleic acid molecules comprises rolling circle amplification (RCA).

In an embodiment, the step of amplifying the ligated nucleic acid molecules comprises Polymerase chain reaction (PCR) amplification.

In an embodiment, the step of amplifying the ligated nucleic acid molecules comprises inhibiting the partially hybridized probe/nucleic acid molecule complexes from being amplified.

In an embodiment, the step of amplifying the ligated nucleic acid comprises first ligating an oligomer assembly to the ligated nucleic acid, wherein the oligomer assembly extends the length of the ligated nucleic acid molecules so as to form an extended ligated nucleic acid molecules, preferably wherein the extended ligated nucleic acid molecules are immobilized.

In an embodiment, the oligomer assembly contains multiple copies of the same sequence.

In an embodiment, the ligation of the oligomer assembly to the ligated nucleic acid enables degradation of the entire oligomer assembly complex, unless the ligated nucleic acid molecule is exonuclease-resistant.

In an embodiment, degradation of the oligomer assembly amplifies the detectable signal from ligated nucleic acid molecules that are complementary to a sequence that differs from the reference sequence.

In an embodiment, degrading of the oligomer assembly complex results in the formation of a single-strand DNA of a known orientation. In an embodiment, the single-strand of DNA contains multiple copies of the same sequence corresponding to a sequence of the oligomer assembly. In an embodiment, the single strand of DNA can be hybridized and sequenced in situ. In an embodiment, the single strand of DNA is hybridized to primer molecules linked to magnetic nanoparticles to magnetize the cell for cell purification.

In an embodiment, the oligomer assembly is formed by using well, condition, or batch specific monomer sequences that can be grown subsequently using further monomer sequences of alternate sequences for combinatorial labeling of the ligated nucleic acid, preferably wherein the oligomer assembly for combinatorial labeling can be used to multiplex 100 to 1,000,000 single cells or wells, or can be used in high-throughput bulk DNA sequencing.

In an embodiment, 50% of the primer molecules are hybridized within two minutes;

In an embodiment, the reaction temperature of step (b) is about 37° C. In an embodiment, the reaction temperature of step (b) is 37° C.

In an embodiment, the ligating of step (b) is ligation with PBCV ligase. In an embodiment, the ligating of step (b) is ligation with T4 Rnl2. In an embodiment, the ligating of step (b) is ligation with T4 DNA ligase.

In an embodiment, in step (b) partially hybridized probes are ligated to adjacent primer molecules at a rate such that they comprise less than 1% of ligated nucleic acid molecules.

In an embodiment, the method can detect the presence of variant ribonucleic acids with a variant allele frequency (VAF) of less than 5%, less than 4%, less than 3%, less than 2%, or about 1%;

In an embodiment, the sensitivity of the method to detect variant ribonucleic acid molecules is 75%-90%;

In an embodiment, the method is conducted ex vivo. In an embodiment, the method is conducted in vitro. In an embodiment, the method is conducted in situ.

In an embodiment, the population of ribonucleic acid molecules is in a tissue culture. In an embodiment, the population of ribonucleic acid molecules are bound to a solid support such as a bead. In an embodiment, the population of ribonucleic acid molecules are bound to parts of a cell. In an embodiment, the population of ribonucleic acid molecules is in a fixed cell or tissue.

In an embodiment, the variant ribonucleic acid molecule is associated with functional changes. In an embodiment, the variant ribonucleic acid molecule is associated with disease. In an embodiment, the variant ribonucleic acid molecule is associated with cancer. In an embodiment, the function changes are functional changes affecting protein structure.

In an embodiment, the variant ribonucleic acid molecule is used for cell tracing. In an embodiment, the variant ribonucleic acid molecule is used for cell labeling.

In an embodiment, the presence or absence of multiple variant ribonucleic acid molecules with different reference sequences is determined by simultaneously performing the method on the population of ribonucleic acid molecules using multiple sets of probes and primer molecules that are each designed as described in step (a) based on the different reference sequences of each of the multiple variant ribonucleic acid molecules.

This invention also provides a composition comprising a primer molecule and at least two probes,
(a) wherein the primer molecule and at least two probes are designed to hybridize to target sequences on a ribonucleic acid molecule such that:
   (i) the 5′ end of the primer molecule and the 3′ end of the at least two probes are adjacent when the probe and primer molecule are hybridized to their respective target sequences on the ribonucleic acid molecule; or
   (ii) the 5′ end of the at least two probes and the 3′ end of the primer molecule are adjacent when the probe and primer molecule are hybridized to their respective target sequences on the ribonucleic acid molecule;
(b) wherein the primer molecule:
   (i) has a melting temperature of at least 50° C. when hybridized to its target sequence;
   (ii) if the primer molecule is designed such that the 5′ end of the primer molecule and the 3′ end of the at least two probes are adjacent when the probe and primer molecule are hybridized to their respective target sequences on the ribonucleic acid molecule;
   (iii) comprises nucleotides starting at its 5′ end that are fully complimentary to at least 8 consecutive nucleotides of the target sequence and has a 5′ phosphorylated A or T if the primer molecule is designed such that the 5′ end of the primer molecule and the 3′ end of the at least two probes are adjacent when the probe and primer molecule are hybridized to their respective target sequence on the ribonucleic acid molecule; and
   (iv) comprises nucleotides starting at its 3′ end that are fully complimentary to at least 8 consecutive nucleotides of the target sequence if the primer molecule is designed such that the 3′ end of the primer molecule and the 5′ end of the at least two probes are adjacent when the probe and primer molecule are hybridized to their respective target sequence on the ribonucleic acid molecule:
(c) wherein the at least two probes:
   (i) comprise L+S nucleotides, wherein L+S is 8 to 12, and L is at least 1;
   (ii) differ in sequence from one another at only one nucleotide base along the length of L,
   (iii) are fully complementary to the target sequence of the probe along the length of S;
   (iv) have a 5′ phosphorylated A or T if the at least two probes are designed such that the 5′ end of the at least two probes and the 3′ end of the primer molecule are adjacent when the probes and primer molecule are hybridized to their respective target sequences on the ribonucleic acid molecule.

This invention also provides a kit comprising a primer molecule and at least two probes,
(a) wherein the primer molecule and at least two probes are designed to hybridize to target sequences on a ribonucleic acid molecule such that:
   (i) the 5′ end of the primer molecule and the 3′ end of the at least two probes are adjacent when the probe and primer molecule are hybridized to their respective target sequences on the ribonucleic acid molecule; or
   (ii) the 5′ end of the at least two probes and the 3′ end of the primer molecule are adjacent when the probe and primer molecule are hybridized to their respective target sequences on the ribonucleic acid molecule:
(b) wherein the primer molecule:
   (i) has a melting temperature of at least 50° C. when hybridized to its target sequence;
   (ii) if the primer molecule is designed such that the 5′ end of the primer molecule and the 3′ end of the at least two probes are adjacent when the probe and primer molecule are hybridized to their respective target sequences on the ribonucleic acid molecule;
   (iii) comprises nucleotides starting at its 5′ end that are fully complimentary to at least 8 consecutive nucleotides of the target sequence and has a 5′ phosphorylated A or T if the primer molecule is designed such that the 5′ end of the primer molecule and the 3′ end of the at least two probes are adjacent when the probe and primer molecule are hybridized to their respective target sequence on the ribonucleic acid molecule; and
   (iv) comprises nucleotides starting at its 3′ end that are fully complimentary to at least 8 consecutive nucleotides of the target sequence if the primer molecule is designed such that the 3′ end of the primer molecule and the 5′ end of the at least two probes are adjacent when the probe and primer molecule are hybridized to their respective target sequence on the ribonucleic acid molecule;
(c) wherein the at least two probes:
   (i) comprise L+S nucleotides, wherein L+S is 8 to 12, and L is at least 1;
   (ii) differ in sequence from one another at only one nucleotide base along the length of L,
   (iii) are fully complementary to the target sequence of the probe along the length of S;
   (iv) have a 5′ phosphorylated A or T if the at least two probes are designed such that the 5′ end of the at least two probes and the 3′ end of the primer molecule are adjacent when the probes and primer molecule are hybridized to their respective target sequences on the ribonucleic acid molecule.

In an embodiment, the composition or kit further comprises a ligase. In an embodiment, the ligase is PBCV ligase. In an embodiment, the ligase is T4 Rnl2. In an embodiment, the ligase is T4 DNA ligase.

In an embodiment of the composition or kit, L is 1, 2, 3, 4, 5, 6, 7, or 8. In an embodiment of the composition or kit, L is 3.

In an embodiment, the composition or kit is for use in determining the presence or absence of variant ribonucleic acids in a population of ribonucleic acid molecules.

In an embodiment, the composition or kit comprises probes and primers designed as in (a), (b) and (c) to hybridize to multiple different target sequences such that multiple different target sequences can be interrogated in series or preferably simultaneously.

In an embodiment, the composition or kit comprises an endonuclease. In an embodiment, the composition or kit comprises an exonuclease. In an embodiment, the composition or kit comprises surveyor enzyme. In an embodiment, the composition or kit comprises resolvase. In an embodiment, the composition or kit comprises ssDNA-binding protein. In an embodiment, the exonuclease is Exonuclease I. In an embodiment, the exonuclease is Exonuclease III. In an embodiment, the composition or kit further comprises RNase H and/or an RNase cocktail.

In an embodiment of the composition or kit, the plurality of probes consists of probes complimentary to each respective single base variant along the length of L.

In an embodiment of the composition or kit, the plurality of probes consists of probes complimentary to each possible single base variant along the length of L other than non-actionable sequences, synonymous mutations, non-functional polymorphisms, or mutational patterns not observed in the human population.

In an embodiment of the composition or kit, some or all of the plurality of probes comprise a signal amplification functional group. In an embodiment of the composition or kit, the signal amplification functional group is horseradish peroxidase. In an embodiment of the composition or kit, the signal amplification functional group is alkaline phosphatase. In an embodiment of the composition or kit, the signal amplification functional group is digoxigenin. In an embodiment of the composition or kit, the signal amplification functional group is or fluorescein isothiocyanate (FITC).

In an embodiment of the composition or kit, some or all of the plurality of probes further comprise an amplification sequence. In an embodiment, the amplification sequence is an adapter sequence for circularization and rolling circle amplification (RCA). In an embodiment, the amplification sequence is a sequence for hybridization of a PCR primer.

In an embodiment of the composition or kit, some or all of the plurality of probes further comprise a barcode.

In an embodiment of the composition or kit, some or all of the plurality of probes further comprise an inverted dT.

In an embodiment of the composition or kit, some or all of the plurality of probes further comprise a cleavable terminator. In an embodiment the cleavable terminator is an inosine base.

In an embodiment of the composition or kit, some or all of the plurality of probes further comprise a functional group that accepts and transfers energy from an external source for generation of cytotoxic processes.

In an embodiment of the composition or kit, some or all of the plurality of probes comprise a fluorophore.

In an embodiment of the composition or kit, some or all of the plurality of probes further comprise a cleavable terminator and Endonuclease V is used to cleave the terminator of the ligated nucleic acid molecule.

In an embodiment of the composition or kit, probes that are fully complimentary to the reference sequence of the variant ribonucleic acid molecules do not further comprise a signal amplification functional group.

In an embodiment of the composition or kit, probes that are fully complimentary to the reference sequence of the variant ribonucleic acid molecules do not further comprise an amplification sequence.

In an embodiment of the composition or kit, probes that are fully complimentary to the reference sequence of the variant ribonucleic acid molecules are tagged with a non-functional control sequence, which control sequence blocks amplification of the ligated nucleic acid molecule.

In an embodiment of the composition or kit, probes that are fully complimentary to the reference sequence of the variant ribonucleic acid molecules comprise an inverted dT to prevent circularization and rolling circle amplification.

In an embodiment of the composition or kit, probes that are fully complimentary to non-actionable sequences, synonymous mutations, non-functional polymorphisms, and/or mutational pattern not observed in the human population do not further comprise a signal amplification functional group.

In an embodiment of the composition or kit, probes that are fully complimentary to non-actionable sequences, synonymous mutations, non-functional polymorphisms, and/or mutational pattern not observed in the human population do not further comprise an amplification sequence.

In an embodiment of the composition or kit, probes that are fully complimentary to non-actionable sequences, synonymous mutations, non-functional polymorphisms, and/or mutational pattern not observed in the human population are tagged with a non-functional control sequence, which control sequence blocks amplification of the ligated nucleic acid molecule.

In an embodiment of the composition or kit, probes that are fully complimentary to non-actionable sequences, synonymous mutations, non-functional polymorphisms, and/or mutational pattern not observed in the human population comprise an inverted dT to prevent circularization and rolling circle amplification.

In an embodiment of the composition or kit, at least 8 consecutive nucleotides starting at the 5′ end of each primer molecule, are fully complimentary to consecutive nucleotides of the reference sequence. In an embodiment of the composition or kit, at least 9 consecutive nucleotides starting at the 5′ end of each primer molecule, are fully complimentary to consecutive nucleotides of the reference sequence. In an embodiment of the composition or kit, at least 10 consecutive nucleotides starting at the 5′ end of each primer molecule, are fully complimentary to consecutive nucleotides of the reference sequence. In an embodiment of the composition or kit, at least 11 consecutive nucleotides starting at the 5′ end of each primer molecule, are fully complimentary to consecutive nucleotides of the reference sequence. In an embodiment of the composition or kit, at least 12 consecutive nucleotides starting at the 5′ end of each primer molecule, are fully complimentary to consecutive nucleotides of the reference sequence. In an embodiment of the composition or kit, at least 13 consecutive nucleotides starting at the 5′ end of each primer molecule, are fully complimentary to consecutive nucleotides of the reference sequence. In an embodiment of the composition or kit, at least 14 consecutive nucleotides starting at the 5′ end of each primer molecule, are fully complimentary to consecutive nucleotides of the reference sequence. In an embodiment of the composition or kit, at least 15 consecutive nucleotides starting at the 5′ end of each primer molecule, are fully complimentary to consecutive nucleotides of the reference sequence.

In an embodiment of the composition or kit, at least 8 consecutive nucleotides starting at the 3′ end of each primer molecule, are fully complimentary to consecutive nucleotides of the reference sequence. In an embodiment of the composition or kit, at least 9 consecutive nucleotides starting at the 3′ end of each primer molecule, are fully complimentary to consecutive nucleotides of the reference sequence. In an embodiment of the composition or kit, at least 10 consecutive nucleotides starting at the 3′ end of each primer molecule, are fully complimentary to consecutive nucleotides of the reference sequence. In an embodiment of the composition or kit, at least 11 consecutive nucleotides starting at the 3′ end of each primer molecule, are fully complimentary to consecutive nucleotides of the reference sequence. In an embodiment of the composition or kit, at least 12 consecutive nucleotides starting at the 3′ end of each primer molecule, are fully complimentary to consecutive nucleotides of the reference sequence. In an embodiment of the composition or kit, at least 13 consecutive nucleotides starting at the 3′ end of each primer molecule, are fully complimentary to consecutive nucleotides of the reference sequence. In an embodiment of the composition or kit, at least 14 consecutive nucleotides starting at the 3′ end of each primer molecule, are fully complimentary to consecutive nucleotides of the reference sequence. In an embodiment of the composition or kit, at least 15 consecutive nucleotides starting at the 3′ end of each primer molecule, are fully complimentary to consecutive nucleotides of the reference sequence.

In an embodiment of the composition or kit, the primer molecule comprises 20-50 nucleotides. In an embodiment of the composition or kit, 20-50 nucleotides that are complementary to the reference sequence of the ribonucleic nucleic acid molecules.

In an embodiment of the composition or kit, each primer molecule further comprises an amplification sequence. In an embodiment of the composition or kit, each primer molecule further comprises a signal amplification functional group. In an embodiment of the composition or kit, each primer molecule further comprises a blocking group to make the ligated nucleic acid molecules resistant to degradation. In an embodiment of the composition or kit, each primer molecule further comprises a fluorescent or colorimetric sequence. In an embodiment of the composition or kit, each primer molecule further comprises an inverted dT. In an embodiment of the composition or kit, each primer molecule further comprises. In an embodiment of the composition or kit, each primer molecule further comprises a phosphorothioate nucleotide or phosphorothioate nucleotides. In an embodiment of the composition or kit, each primer molecule further comprises a locked nucleic acid or locked nucleic acids. In an embodiment of the composition or kit, each primer molecule further comprises a modified base or modified bases. In an embodiment of the composition or kit, each primer molecule further comprises a functional group that accepts and transfers energy from an external source for generation of cytotoxic processes.

In an embodiment of the composition or kit, each primer molecule comprises a blocking group to make the ligated nucleic acid molecules resistant to degradation. In an embodiment the blocking group is an inverted dT.

In an embodiment the blocking group is a phosphorothioate nucleotide or phosphorothioate nucleotides. In an embodiment the blocking group is an inert spacer moiety. In an embodiment the blocking group is a locked nucleic acid or locked nucleic acids. In an embodiment the blocking group is a modified base or modified bases.

In an embodiment each primer molecule comprises an amplification sequence. In an embodiment the amplification sequence is an adapter sequence for circularization and rolling circle amplification (RCA). In an embodiment the amplification sequence is a sequence for hybridization of a PCR primer.

This invention also provide a composition comprising complexes of primer molecules, probes and ribonucleic acid molecules,
(a) wherein the complexes comprise primer molecules and probes that are hybridized to target sequences on the ribonucleic acid molecules such that:
   (i) the 5′ end of the primer molecules and the 3′ end of the probes are adjacent when hybridized their respective target sequences on the ribonucleic acid molecules; or
   (ii) the 5′ end of the at least two probes and the 3′ end of the primer molecule are adjacent when hybridized their respective target sequences on the ribonucleic acid molecules:
(b) wherein the primer molecules:
   (i) have a melting temperature of at least 50° C. when hybridized to their target sequence;
   (ii) comprise nucleotides that are fully complimentary to at least 8 consecutive nucleotides of the target sequence at the 5′ end of the primer molecule and have a 5′ phosphorylated A or T if the primer molecules are hybridized to their target sequence on the ribonucleic acid molecules such that the 5′ end of the primer molecules and the 3′ end of the probes are adjacent; and
   (iii) comprise nucleotides that are fully complimentary to at least 8 consecutive nucleotides of the target sequence at the 3′ end of the primer molecule if the primer molecules are hybridized to their target sequence on the ribonucleic acid molecules such that the 3′ end of the primer molecules and the 5′ end of the probes are adjacent; and
(c) wherein the composition comprises at least two probes, wherein such probes:
   (i) comprise L+S nucleotides, wherein L+S is 8 to 12, and L is at least 1;
   (ii) differ in sequence from one another at only one nucleotide base along the length of L,
   (iii) are fully complementary to the target sequence of the at least two probes along the length of S;
   (iv) have a 5′ phosphorylated A or T if the probes are hybridized to their target sequence on the ribonucleic acid molecules such that the 3′ end of the primer molecules and the 5′ end of the probes are adjacent.

In an embodiment, the complexes further comprise a ligase. In an embodiment, the composition of complexes comprises the complexes formed by performing the methods described herein.

This invention also provides method of treating a disease or condition associated with the presence of variant ribonucleic acid molecules in a subject, the method comprising:
(a) using any of the methods described herein to determine the presence or absence of a variant ribonucleic acid molecule in the subject;
(b) treating the subject based on the presence or absence of the variant ribonucleic acid molecule.

In an embodiment, the subject is a human. In an embodiment, the subject is not a human.

The present invention also provides for methods, processes, compositions, devices, and kits for practicing substantially what is shown and described.

Each embodiment disclosed herein is contemplated as being applicable to each of the other disclosed embodiments. Thus, all combinations of the various elements described herein are within the scope of the invention.

### Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, “about” in the context of a numerical value or range means±10% of the numerical value or range recited or claimed, unless the context requires a more limited range.

The terms “sequencing primer” and “primer molecule” are used interchangeably herein. As used herein, the “primer molecule” encompasses both (a) the nucleotides that are fully complementary to ribonucleic acid molecules in the population of ribonucleic acid molecules, and the (b) any other component that is covalently attached to these nucleotides, such as, without limitation, additional nucleotides that are partially complementary to the ribonucleic acid molecules, additional nucleotides that are complementary to PCR primers for subsequent amplification, additional nucleotides that block exonuclease digestion, spacers, signal amplification functional groups, or other functional groups. The nucleotides of the primer molecule that are fully complementary to ribonucleic acid molecules in the population of ribonucleic acid molecules are preferably deoxyribonucleotides.

The terms “template”, “nucleic acid”, and “nucleic acid molecule”, are used interchangeably herein, and each refers to a polymer of nucleotides. “Nucleotide” shall mean any monomer units for forming the deoxyribonucleic acids and ribonucleic acids or derivatives or analogues thereof, or hybrids of any of these. These monomer units include, without limitation, deoxyribonucleotides and ribonucleotides, nucleotides that have been modified according to techniques known in the art, and the monomer units of nucleic acid analogues. “Nucleic acid analogues” are structural analogues of DNA or RNA, designed to hybridize to complementary nucleic acid sequences. Examples of nucleic acid analogs include, but are not limited to the Nucleic acid analogues disclosed in Hunziker, J, and Leumann, C. (1995), peptide nucleic acids (PNA), locked nucleic acids (LNA) (Imanishi, et al WO 98/39352; Imanishi, et al WO 98/22489; Wengel, et al WO 00/14226), 2′-O-methyl nucleic acids (Ohtsuka, et al, U.S. Pat. No. 5,013,830), 2′-fluoro nucleic acids, phosphorothioates, and metal phosphonates. The term “nucleotide base” may be used interchangeably with “nucleotide”. “Genomic nucleic acid” refers to DNA derived from a genome, which can be extracted from, for example, a cell, a tissue, a tumor or blood.

As used herein, the term “amplifying” refers to the process of synthesizing nucleic acid molecules that are complementary to one or both strands of a template nucleic acid. Amplifying a nucleic acid molecule typically includes denaturing the template nucleic acid, annealing primers to the template nucleic acid at a temperature that is below the melting temperatures of the primers, and enzymatically elongating from the primers to generate an amplification product. The denaturing, annealing and elongating steps each can be performed once. Generally, however, the denaturing, annealing and elongating steps are performed multiple times (e.g., polymerase chain reaction (PCR)) such that the amount of amplification product is increasing, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. An “amplification sequence” is a sequence of nucleotides whose presence is necessary to amplify a nucleic acid molecule using a given amplification method, such as, without limitation, an adapter sequence for rolling circle amplification (RCA), or a sequence which PCR primers may hybridize to for PCR amplification.

As used herein the term “amplicon” refers to a nucleic acid molecule that is the product of amplifying a nucleic acid molecule.

As used herein the term “multiple displacement amplification” (MDA) refers to a method of isothermal, strand-displacing amplification as described in Dean et al. 2002.

As used herein, the term “sequence” may mean either a strand or part of a strand of nucleotides, or the order of nucleotides within a strand or part of a strand, depending on the appropriate context in which the term is used. Unless specified otherwise in context, the order of nucleotides is recited from the 5′ to the 3′ direction of a strand.

As used herein, the term “read” or “sequence read” refers to the nucleotide or base sequence information of a nucleic acid that has been generated by any sequencing method. A read therefore corresponds to the sequence information obtained from one strand of a nucleic acid fragment. For example, a DNA fragment where sequence has been generated from one strand in a single reaction will result in a single read. However, multiple reads for the same DNA strand can be generated where multiple copies of that DNA fragment exist in a sequencing project or where the strand has been sequenced multiple times. A read therefore corresponds to the purine or pyrimidine base calls or sequence determinations of a particular sequencing reaction.

As used herein, the terms “sequencing”, “obtaining a sequence” or “obtaining sequences” refer to obtaining nucleotide sequence information that is sufficient to identify or characterize the nucleic acid molecule and could be the full length or only partial sequence information for the nucleic acid molecule.

As used herein, the terms “wild-type” or “reference sequence” refers to a non-mutant sequence of nucleotides from a genome of the same species as that being analyzed, for which genome at least the non-mutant sequence information is known. As used herein, the term “wild-type” may be used interchangeably with “reference”. Reference sequence may refer to a non-mutant ribonucleotide sequence.

In embodiments of the present invention, “having a known nucleotide sequence” may refer to having a known “reference nucleotide sequence.”

As used herein, the term “variant” or “variant allele” refers to a sequence of nucleotides, variant codon, or indel, resulting in a sequence other than a wild-type sequence from the genome of the same species as that being analyzed for which genome the non-mutant sequence information is known. As used herein, the term “variant allele frequency” (VAF) refers to the refers to the ratio of variant alleles to wild-type alleles in a population. For example, 1 variant allele among 1,000,000 wild type alleles may be represented as a 10⁻⁶ VAF. In embodiments of the present invention, the VAF may be less than about 10⁻², 10⁻³, 10⁻⁴, 10⁻⁵, 10⁻⁶, 10⁻⁷, 10⁻⁸. In embodiments of the present invention the VAF may be less than 10⁻⁹. As used herein, “variant allele” may refer to the variant allele in the genome or the variant allele that has been transcribed into a variant ribonucleic acid molecule. As used herein “variant ribonucleic acid molecule” is a ribonucleic acid molecule that has a sequence of ribonucleotides other than the ribonucleic acid wild-type sequence.

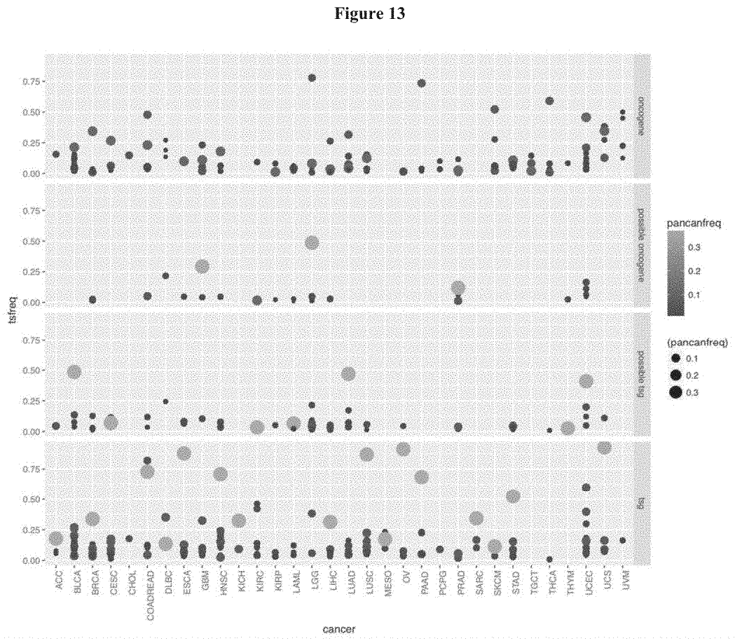

As used herein, the term “functionally relevant sequences” refers to sequences whose alterations could lead to functional changes, diseases, or lend themselves to lineage or cell labeling applications (See e.g., FIG. 13).

As used herein a “functionally relevant sequence variant” refers to the “variant allele” of a functionally relevant sequence. A “variant ribonucleic acid molecule” may be a functionally relevant sequence variant if it encodes a sequence whose alterations could lead to functional changes, diseases, or lend themselves to lineage or cell labeling applications. Thus, “functionally relevant sequence variant” encompasses functionally relevant variant ribonucleic acid molecules.

In embodiments of the present invention, the wild-type allele for a functionally relevant sequence has a known nucleotide sequence. Accordingly, in embodiments of the present invention nucleic acid molecules comprising the wild-type allele of the functionally relevant sequence are preferentially not amplified.

As used herein, the term “saturate” may be used interchangeably with the term “capture”. In embodiments of the present invention, saturating a sequence with, for example probes or primers, comprises saturating the sequence with a concentration of probes or primers capable of saturating the sequence.

In embodiments of the present invention, each probe may differ from the reference sequence at one or more nucleotide base.

In embodiments of the present invention, ligating each probe to a primer in competition under conditions favoring the ligation of fully hybridized probes over partially hybridized probes comprises ligating only hybridized probes.

In embodiments of the present invention, degrading un-ligated, excess, and/or off-target probes comprises removing un-ligated, excess, and/or off-target partially degenerate probes.

In embodiments of the present invention, a mixture of nucleic acid molecules comprising a plurality of functionally relevant sequences may refer to a mixture comprising a plurality of nucleic acid molecules each comprising the same functionally relevant sequence, or comprising a plurality of functionally relevant sequences among the nucleic acid molecules.

As used herein, the term “barcode”, also known as an “index,” refers to a unique DNA sequence within a sequencing adaptor used to identify the sample of origin for each fragment.

As used herein, the term “gene” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins and locus control regions.

As used herein, the term “sequencing target” refers to the sequence of interest which is selected, amplified, and/or revealed via the sequencing operation. This sequence is represented in a traditional format via the oligonucleotide bases (e.g. G, T, A, C, and U) or in a similar textual format. “Target sequences on a ribonucleic acid molecule” are sequences of A, G, U and C nucleotides on the ribonucleic acid molecule that the primer molecules and probes are designed to hybridize to.

As used herein, the term “next generation sequencing” or “NGS” refers to any modern high-throughput sequencing technology. NGS includes, but is not limited to, sequencing technologies such as Illumina (Solexa) sequencing and SOLiD sequencing.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the melting temperature of the formed hybrid, and the G:C ratio within the nucleic acids.

As used herein, the term “probe” refers to an oligonucleotide (i.e., a sequence of nucleotides), whether occurring naturally as in a purified restriction digest or produced synthetically, recombinantly or by amplification (e.g. PCR), which is capable of hybridizing to another oligonucleotide of interest. Probes are useful in the detection, identification and isolation of particular gene sequences (e.g., Her2, marker A1, marker A2 or marker B). The term probe encompasses the oligonucleotide portion of the probe that is designed to hybridize to a target sequence as well as any other any other component that is covalently attached to these nucleotides. For example, it is contemplated that any probe used in the present invention may be labeled with any “reporter molecule,” so that is detectable in any detection system, including, but not limited to enzyme (e.g., ELISA, as well as enzyme-based immunohistochemical assays), fluorescent (e.g., FISH), radioactive, mass spectroscopy, and luminescent systems. It is not intended that the present invention be limited to any particular detection system or label. As used herein the term “k-mer” refers to a probe of length k.

As used herein, the term “oligomer assembly” is used interchangeably with “concatemers”. Concatemers may be formed by short monomers that anneal to one another by virtue of having partially overlapping oligonucleotides.

In embodiments of the present invention, a mixture of nucleic acid molecules comprising a plurality of functionally relevant sequences may comprise nucleic acid molecules comprising the wild-type allele of the functionally relevant sequence and/or nucleic acid molecules comprising the variant allele, variant codon, or indel for the functionally relevant sequence. A “population of ribonucleic acid molecules” may comprise ribonucleic acid molecules comprising the wild-type/reference sequence of the functionally relevant sequence and/or ribonucleic acid molecules comprising the variant ribonucleic acid molecule. A “population of ribonucleic acid molecules” may refer to any composition that comprises ribonucleic acid molecules, such as, without limitation, a cell, a tissue, a tumor or blood.

As used herein, the term “Tₘ” refers to the melting point of a nucleic acid template, measured as the temperature(s) at which half of the nucleic acid template is present in a single-stranded (denatured) form.

As used herein, the term “T_{reaction}” refers to the temperature(s) at which a hybridization reaction is being conducted.

### General Overview

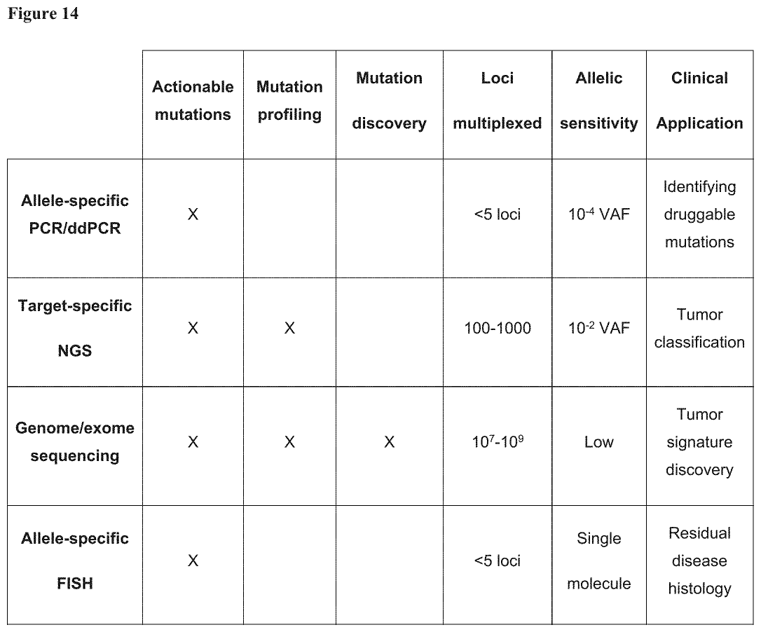

The human body has trillions of cells, excluding microbial organisms, and each cell has a unique combination of gene expression, somatic mutation, epigenetic modification, and post-transcriptional processing. If cells could be labeled using genomic signatures with single-nucleotide specificity and sensitivity, it might be possible to map functional genetic mosaicism and/or distinguish aberrant from normal cells early in complex traits disease progression and use this information for disease screening or in early detection, such as cancer. (FIG. 14).

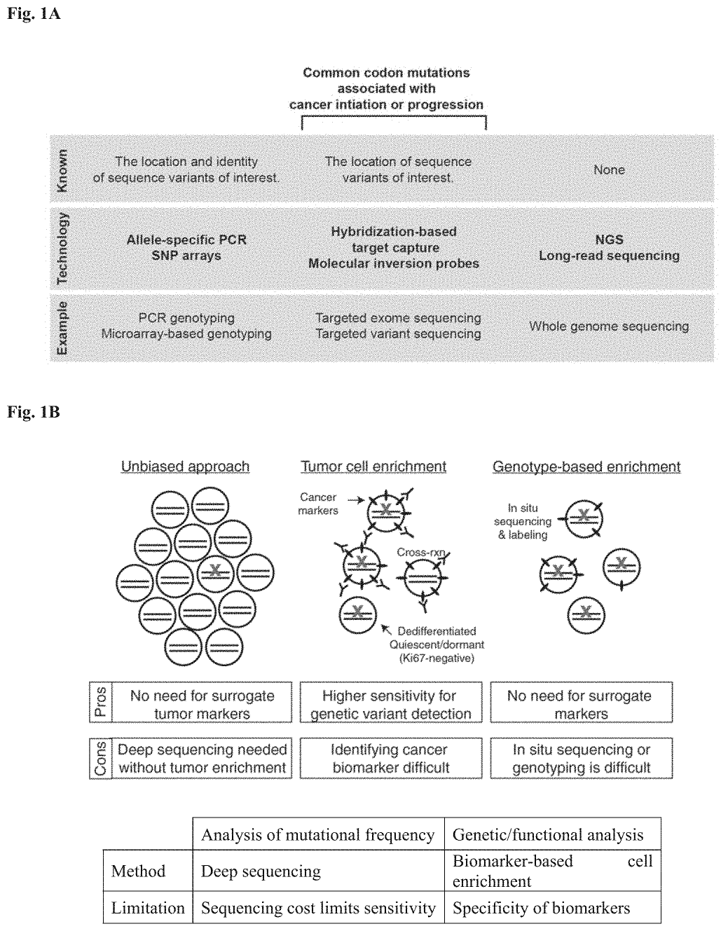

The present invention discloses an algorithm and reaction parameters to reduce the degenerate probe complexity in DNA or RNA or nucleic acid sequencing, and its application in single cells for highly accurate consensus base calling using a wide range of enzymes and conditions. The algorithm of the present invention for probe selection favorably impacts the detection of rare cancer cells in an affordable and scalable manner, compared to traditional sequencing or single-cell quantification methods (FIG. 14). In addition, the present invention enables a wider range of probe barcoding, modifications, signal detection approaches in single cells (FIG. 1A).

Embodiments of the present invention disclose a method for quantifying or labeling single cells based on RNA-templated in situ sequencing chemistry, overcoming barriers with regard to sequencing and the detection sensitivity, specificity, bias, speed, scalability, and read-length for sequencing RNA molecules directly, i.e. RNA-seq, in single cells for massively parallel single-cell analysis, image-based functional genomics, and cancer diagnostics (FIG. 1A).

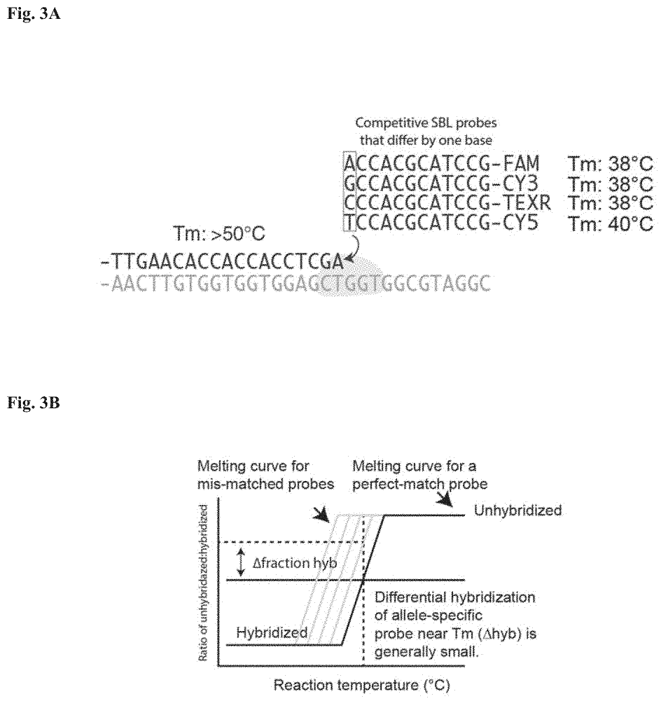

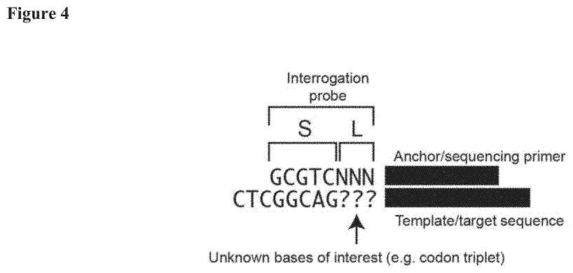

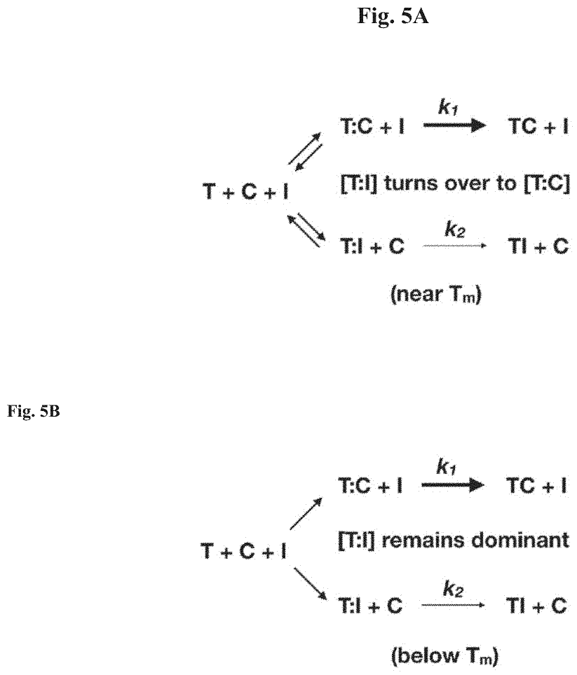

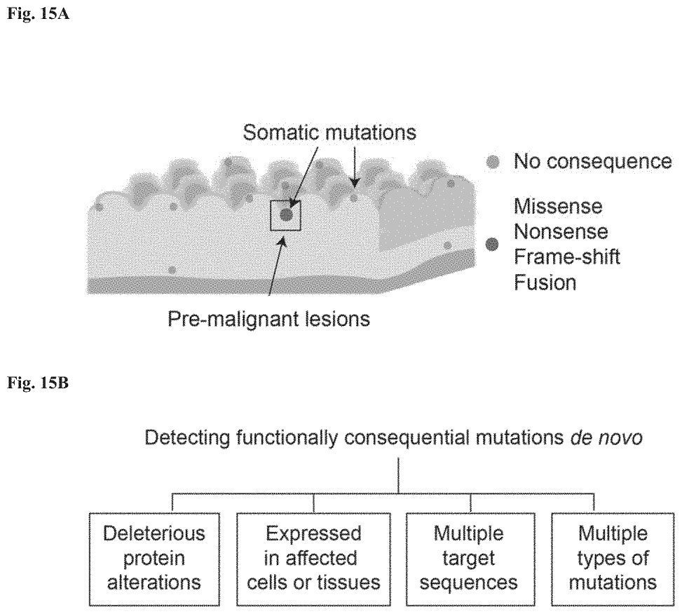

Embodiments of the present invention describe methods for sequencing a subset of RNA or nucleic acid sequences from any given loci using DNA ligase-dependent primer extension methods. The present invention enables one to choose the desired sequencing product (e.g. variant base compositions or positions) versus indiscriminately interrogating all possible sequence variants. By selecting a set of oligonucleotides containing mixed bases to interrogate functionally relevant subsequences, while ignoring uninformative or background sequences (FIG. 15A), embodiments of the present invention reduce the complexity of interrogating oligonucleotides, thereby significantly increasing and enabling the fidelity, sensitivity, detection, and kinetics of a sequencing reaction in a predictable manner for cost-effective and sensitive sequencing and detection of mutations in rare single cells bearing de novo mutations with functional significance (FIG. 3B).

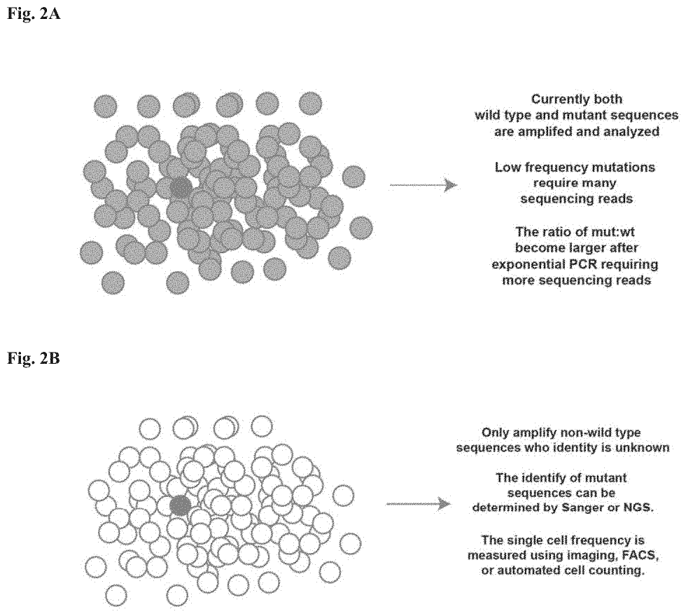

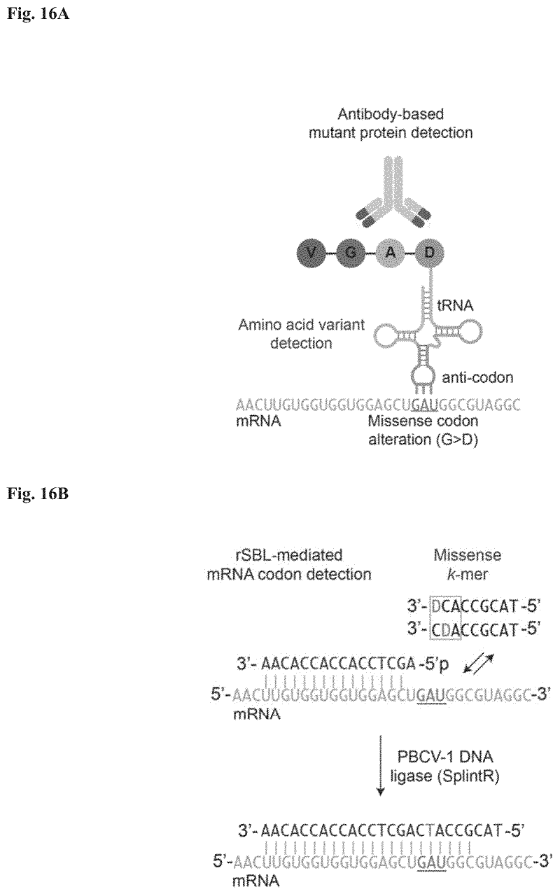

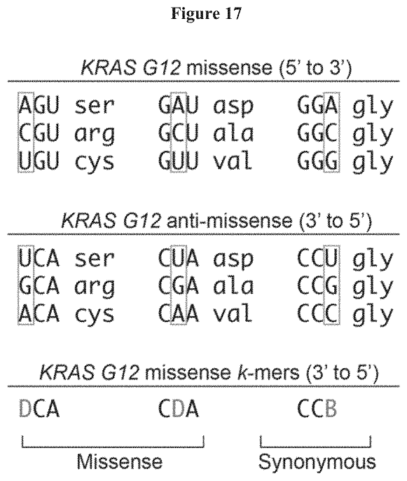

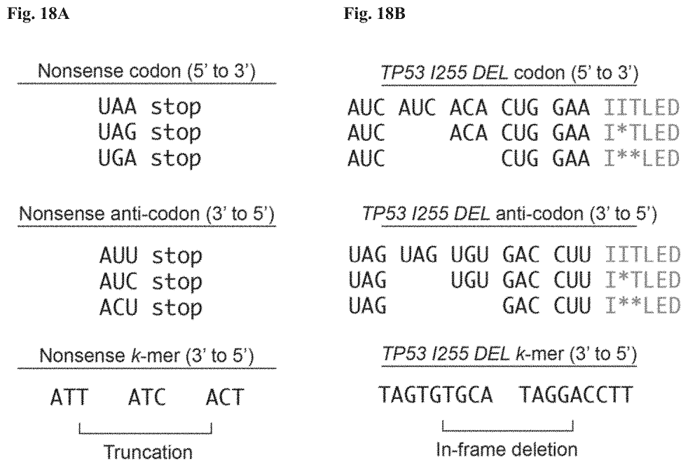

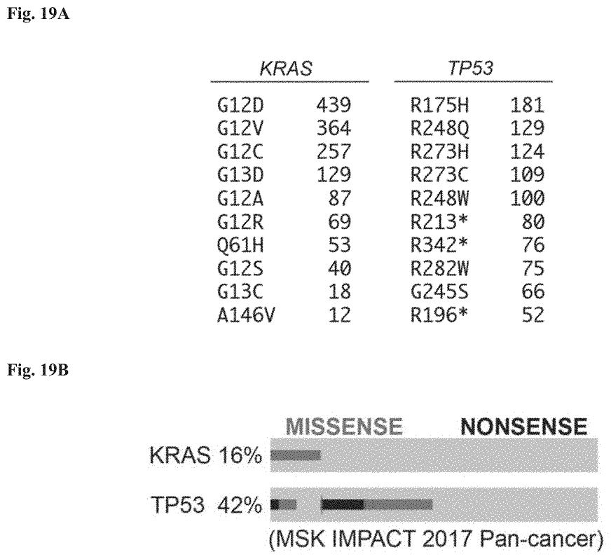

Embodiments of the present invention may utilize sequencing probes capable of only detecting a subset of relevant sequences (FIG. 2B). When combined with in situ single cell-resolution or optical imaging, embodiments of the present invention reduce the sequencing depth necessary to detect rare sequence variants and filters out false positives with high accuracy. One major benefit of this approach is that it can be applied to intact single-cell or tissues for in situ applications to visualize and sort single cells for a wide range of clinical applications. Another major benefit of this approach is the ability to bypass need for antibodies (FIG. 16A) for detecting codon changes that alter the amino acid composition in the protein (FIG. 16B). For example, the identification of, e.g., disseminated cancer cells relies or is dependent on general tissue stains and functional or cellular biomarkers; however, most disseminated cancer cells and many cancer cells remain invisible or indiscernible due to the lack of robust biomarkers. Embodiments of the present invention render visible or detect previously invisible cancer cells, including in situ, with a major clinical significance (e.g. tumor metastasis) in situ for single-cell analysis. Accordingly, in embodiments of the present invention, sequencing targets comprise one or more variant alleles or codons of a functionally relevant sequences. (FIGS. 17, 18, and 19).

The primer molecules have a melting temperature of at least 50° C. when hybridized to their complimentary ribonucleic acid molecules in the population of ribonucleic acid molecules. In embodiments the melting temperature primer molecules may be about 50, 51, 52, 53, 54, 55, 56, 57, 58, or 58° C. or may be at least 60° C. An aspect of the invention is a large differential between the melting temperature of the primer molecules compared to the probes when hybridized to their respective complimentary sequences. Probes that are fully complementary along the length of L+S have a melting temperature that is about the same as the reaction temperature, such that probes that have a mismatch along the length of L have a melting temperature that is below the reaction temperature. Since the reaction temperature is well below the melting temperature of the primer molecules, the primer molecules are fully hybridized to their reference sequences on the ribonucleic acid molecules during the ligation reaction.

In embodiments of the present invention, each sequencing target is comprised of a constant primer region, either upstream or downstream of a variable region of length L to be interrogated. The variable region or the sequence within the interrogated region may be as short as one (1)-base, three-(3) bases (e.g. codon), or longer (e.g. insertion, deletion, splicing enhancers, protein binding motifs, junction, fusion, molecular barcodes); however, L is generally, and in some embodiments always, less than the sequencing read-length. In a particularly useful embodiment of the invention, the probes are designed to interrogate three-(3) bases that form a codon (anti-codon oligonucleotides) to determine the presence or absence of variant ribonucleic acid molecules that produce a functional change in a protein that is translated from the ribonucleic acid molecule.

The length (L) determines the total number of possible sequences (4^{L}) that could be expected at random, i.e. if every base is uniformly degenerate. There is one wild-type or reference sequence for a given locus, i.e. for each locus (FIG. 3A). Therefore, the number of alternative, i.e. non-wild-type sequences that may be sequenced is 4^{L}−(minus) 1. When L=12, or ˜equals twelve, greater than 16.8 million, i.e. 777, 215 or 4²−1 non-wild-type sequences are possible.

The overwhelming number of somatic mutational events are due to point mutations, resulting from errors in DNA replication or repair. They occur at the rate of ˜1 to 10 per cell division from embryonic to adult development (Bae, T. et al. (2017); Lodato, M. A., et al. (2018)). Hundreds to thousands of somatic point mutations are present in single cells (Bae, T. et al. (2017); Lodato, M. A., et al. (2018); Enge, M. et al. (2017); Navin, N., et al. (2011)). The low frequency of somatic mutations and the large size of the human genome makes it unlikely that any two independent point mutations occur within the span of L bases in a single cell. If one considers single point mutations or point mutations across or in L, only 3L sequences are possible, that is to say the number of non-wild-type sequences is much smaller (3L). When L=12 (equals twelve), 36 non-wild-type sequences exist, i.e. this value is 36, nearly a 470,000-fold (466,000-fold) reduction in potential sequences and therefore interrogation probe complexity [(4^{L}−1)/3L]. It is possible to represent all oligonucleotide, e.g. three oligonucleotide, sequences containing a single or all possible point mutations and their complementary sequences as a set of n oligonucleotides using mixed bases that excludes the wild-type base at each position, as shown in Table 1 below. Here, n is equal to L for single point mutations. When L=equals 12, twelve (12) synthetic mixed-base oligonucleotides can represent all possible point or single point mutation sequences.

TABLE 1
Interrogating mixed bases for each wild type base

| WT codon | Mutant codon | Complement to mutant |
|---|---|---|
| base | base | base |
| A | G, C, or T (B) | C, G, or A (V) |
| G | A, C, or T (H) | T, G, or A (D) |
| C | A, G, or T (D) | T, C, or A (H) |
| T | A, G, or C (V) | T, C, or G (B) |

Compared to standard NGS, in which all bases are degenerate, programmable k-mers have 105-fold lower sequence complexity and permit higher molar concentration per sequence for SBL.

In embodiments of the present invention the sequence space can be further reduced to ignore non-informative sequences, including synonymous mutations, non-functional polymorphisms, and unobserved mutational patterns, for example mutational patterns not observed in human diseases, by changing the mixed base symbols among the in oligonucleotides. In embodiments of the present invention n remains unchanged as long as all base positions can be mutated. Therefore, a set of n oligonucleotides can interrogate any sequence subspace containing a single point mutation. In embodiments of the present invention n oligonucleotides can interrogate any sequence subspace containing a single point mutation using oligonucleotide extension methods (e.g. Sequencing-By-Ligation).

Sequencing-By-Ligation (SBL) interrogates multiple contiguous bases at once, with a variable base calling accuracy (Landegren, U., et al. (1988); Shendure, J. et al. (2005)). In embodiments of the present invention, variable base calling accuracy is achieved by decreasing ligation further away from the ligation junction. Therefore, the ligation specificity and reaction conditions are important parameters for determining the allowable value of L (FIG. 3A). Allele-specific hybridization alone is highly susceptible to hybridization temperature changes (FIG. 3B).

In SBL, the sequencing template (hereinafter T), a DNA template, is pre-hybridized to the sequencing primer (SP) and is transiently bound to interrogating oligonucleotides of length L+S (FIG. 4), in which S is additional non-degenerate bases complementary to T and L is a variable region complementary to potential variant sequence nucleotides. In embodiments of the present invention, the sequencing primer (SP) is pre-hybridized prior to the addition of interrogating oligonucleotides of length L. In such embodiments, L is the length of a region-of-interest (functionally relevant sequence) potentially containing a functionally deleterious variant sequence (i.e. functionally relevant sequence variant). Here, interrogating oligonucleotides containing one or more mismatches (hereinafter I or M, or MM, i.e. k-mer_{mismatch}) compete with perfectly complementary or matching interrogating oligonucleotides (hereinafter C or PM, i.e. k-mer_{perfectmatch}). If every unique sequence is present at an equal molar concentration in solution, the relative amount or concentration ratio of C (PM, k-mer_{perfectmatch}) to M (MM, k-mer_{mismatch} is 1/(4^{L}−1)). As used herein C, PM and k-mer_{perfectmatch} may be used interchangeably. As used herein, I, M, or MM may be used interchangeably.

Because base-pairing mismatches incur ΔG° penalties, the ratio of T:M to T:C intermediate or MM: k-mer_{mismatch} to SP: k-mer_{perfectmatch} pre-ligation complexes are determined by K_{eq} of hybridization, which can be inferred from ΔΔG° between T:M and T:C, i.e. the two possibilities. The ΔG° penalty for a single-base mismatch is +0.5 kcal/mol, whereas a correct pair lowers ΔG° by −1.3 kcal/mol. When T_{reaction} is equal to Tₘ, the amount of T:C, i.e. k-mer_{perfectmatch}, is ˜50% more than T:M for a 1-base k-mer_{mismatch} based on ΔΔG° i.e. for single base mismatch (Zhang, D. Y. et al. (2015); Wang, J. S., et al. (2015)) calculations (FIG. 3B).

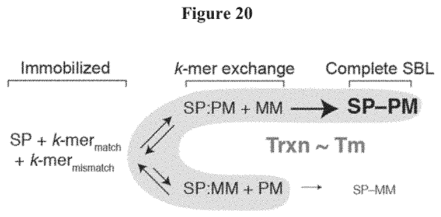

If T_{reaction}<<Tₘ, the ratio of T:C (SP: k-mer_{perfectmatch}) to T:M (SP: k-mer_{mismatch}) is dictated by the initial concentration of C and MM, their probe concentrations since they do not equilibrate (FIG. 20). Since MM k-mer_{mismatch} is a pool of oligonucleotides containing single-nucleotide mismatches, the molar ratio of MM relative amount of C (SP. k-mer_{perfectmatch}) can be as very low or vanishingly small when L is large. In fact, >92% of the ligation-ready hybridized oligonucleotide complex will contain one (1) mismatch if T_{reaction}<<Tₘ and L=12, which leads to low ligation efficiency and frequent base mis-incorporation.

Not all possible k-mer sequences for a given locus will have the same Tₘ, for example depending on their GC content. However, it is impractical to calculate Tₘ for each possible sequence across the sequencing loci, or for every sequencing locus, for optimal probe hybridization. This is the reason why allele-specific primer hybridization (e.g. PCR, FISH) cannot be multiplexed, since each target-specific primer could have different Tₘ that exceeds the Tₘ differences between alleles.

Therefore, purely hybridization-based methods for sequencing are prone to error as the number of degenerate probes increases (or in multiplexing experiments), even if T_{reaction} approaches the average Tₘ(FIG. 3B). The low initial concentration (¼^{L}) of C(SP: k-mer_{perfectmatch}) requires a much longer hybridization time to reach the equilibrium of correctly hybridized molecules.

To overcome the extremely low concentration of C (SP: k-mer_{perfectmatch}) (SP:PM in FIG. 20) as L becomes large and to speed up the sequencing reaction and increase SBL efficiency, one can drive the reaction kinetically rather than relying on the thermodynamic equilibrium alone. For example, one can utilize differences in reaction kinetics in addition to thermodynamic considerations. Here, the reaction speed is defined by the turnover speed (rate) of DNA ligation, specifically unidirectionally. In embodiments of the present invention the reaction speed is preferably controlled as long as C and M are provided in excess of T (FIG. 20).

DNA ligases have distinct Kₘ and k_{cat} for T:C (SP:PM) and T:I (SP:MM) complexes. Kₘ describes the affinity for which the enzyme recognizes the substrate, and k_{cat} describes the turnover rate of the substrate once bound to the enzyme. For high-fidelity ligases k_{cat}/Kₘ can be several orders of magnitude larger than k_{cat}/Kₘ of mismatched substrates.

If T:C (SP:PM) and T:M (SP:MM) cannot equilibrate (T_{reaction}<<Tₘ), a large fraction of T:M (SP:MM) will prevent the formation of T:C (SP:PM) for productive ligation, reducing the overall SBL yield. In addition, DNA ligases will eventually ligate T:M as well, increasing the sequencing error rate over time (FIG. 20; FIG. 5).

Therefore, any contiguous bases of length L can be driven to near completion for SBL if DNA ligases demonstrate a measurable k_{cat}/Kₘ difference between T:C (SP:PM) vs. T:M (SP:MM) as long as T:C and T:M (SP:PM vs. SP:MM) continue to equilibrate (T_{reaction}˜Tₘ). This assumes that no other trapped or non-productive products are formed during the reaction. Some ligases and sequence motifs form adenylated DNA products during the reaction, which reduce the concentration of T:C (SP:PM) and also inhibit the activity of DNA ligases. In embodiments of the present invention this will limit the practical efficiency of SBL at any given concentrations or reaction temperatures.

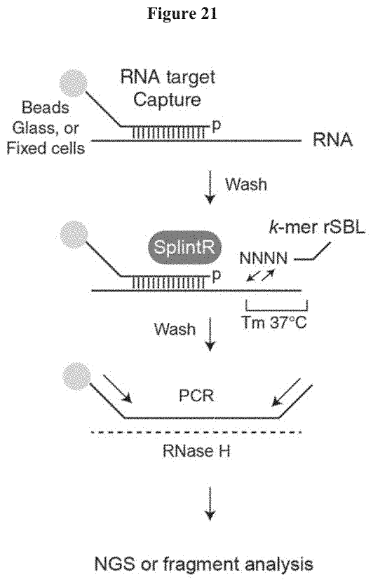

Embodiments of the present invention restrict the probe complexity to detect single point mutations or a specific subset of possible sequence variants (e.g. non-synonymous mutations), thereby increasing the initial relative concentration of C (SP:PM) for ligation reactions (e.g. ˜470,000, specifically, 466,000-fold, for L=12). In embodiments of the present invention the concentration is increased compared to completely degenerate k-mers (FIG. 21). This step increases the fraction of T:C (SP:PM) across a range of templates, temperatures, or conditions, allowing one to sequence contiguous bases using a wider range of DNA ligases. This also allows one to increase L and scan a wide region for point mutations without exponentially reducing the efficiency of SBL. In embodiments of the present invention, the increased efficiency of SBL is retained at suboptimal reaction temperatures. In addition, this also narrows the range of Tₘ so that Tₘ-on can be optimized for a specific DNA ligase of interest.

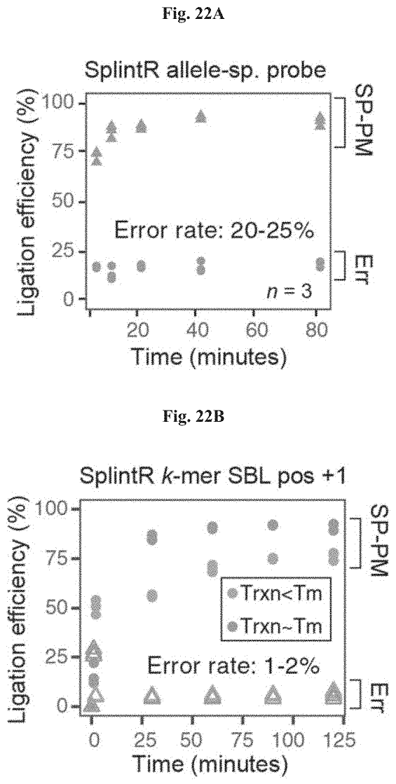

In embodiments of the present invention it is understood that controlling the reaction temperature of SBL and L is important for reducing the error rate and increasing the efficiency. For example, at a reaction temperature below Tₘ of k-mers, the probability of DNA hybridization containing single-base mismatch increases, which forces T4 DNA ligase to ligate incorrect oligonucleotides. In the absence of correct k-mers, the erroneous primer ligation is as high as 25% (FIG. 22A). Even when incorrect k-mers do not ligate, they may remain bound to the template and block correct k-mers from participating in SBL, reducing the efficiency (FIG. 22B). It is understood in embodiments of the present invention, that for DNA ligation at different temperatures, such parameters should be adjusted when designing primers.

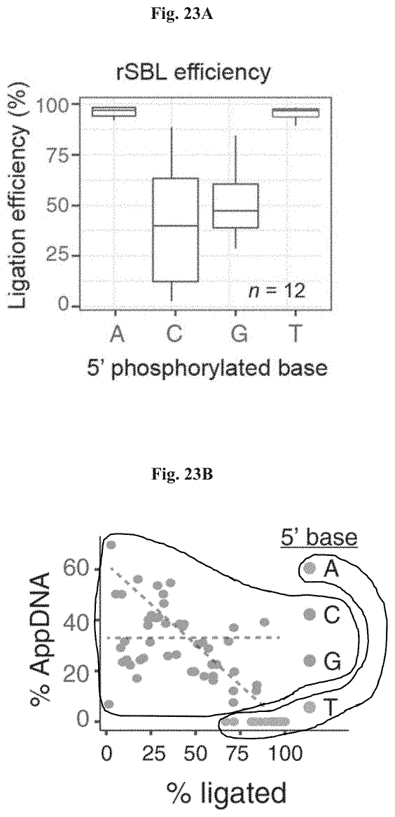

In embodiments of the present invention it is understood that 5′ phosphate base may be critical for high rSBL efficiency (FIG. 23A) and that the low efficiency of rSBL with 5′ phosphate C or G is due to the accumulation of 5′ adenylated DNA (sequencing primer) (FIG. 23B). Embodiments of this invention includes the utilization of sequencing primer design that avoids 5′ C or G, addition of deadenylase in rSBL, or lowering of the ATP concentration to reduce the amount of sequencing primers trapped in the adenylated state.

In embodiments of the present invention SBL can be implemented on DNA or RNA using click chemistry using alkenyl and azide modifications to the sequencing primer and k-mer ends. The reaction condition of click-based DNA ligation is adjusted to maximize the difference of ligation between matched and mismatched k-mer probes.

Embodiments of the present invention provide an SBL product engineered to contain DNA modifications for conditional DNA amplification or elimination. This allows one to selectively amplify any subset of sequences after SBL or degrade wild-type sequences that could interfere with the rare variant detection.

The initial SBL product remains hybridized to the RNA template, forming a DNA-RNA duplex. If error in SBL were to occur, it creates one or more mismatches between DNA and RNA strands. Embodiments of the present invention provide use of an endonuclease, Surveyor enzyme, resolvases, or ssDNA-binding proteins specific for mis-matched ssDNA loops which can recognize such mismatches. This will cleave error containing SBL products so that they cannot be amplified (e.g. enzyme-linked) for highly specific molecular readout (e.g. optical imaging).

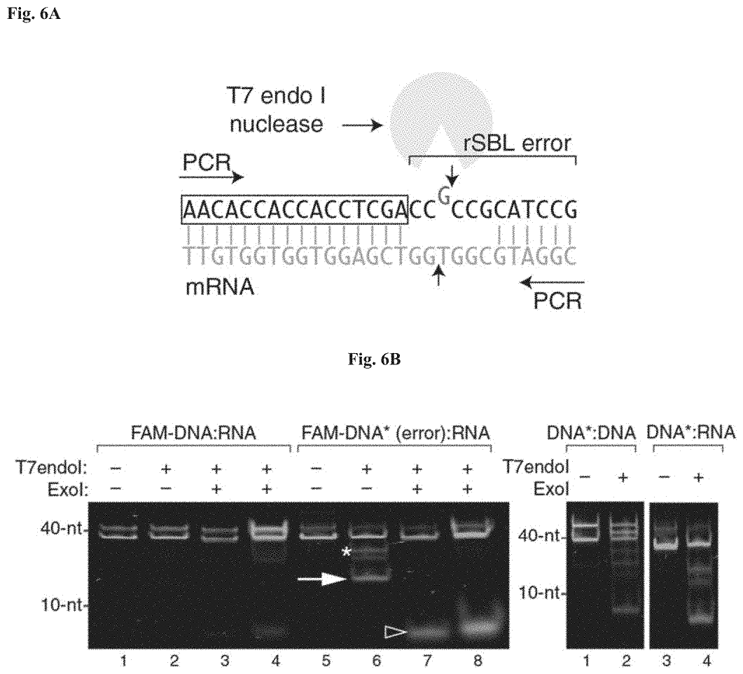

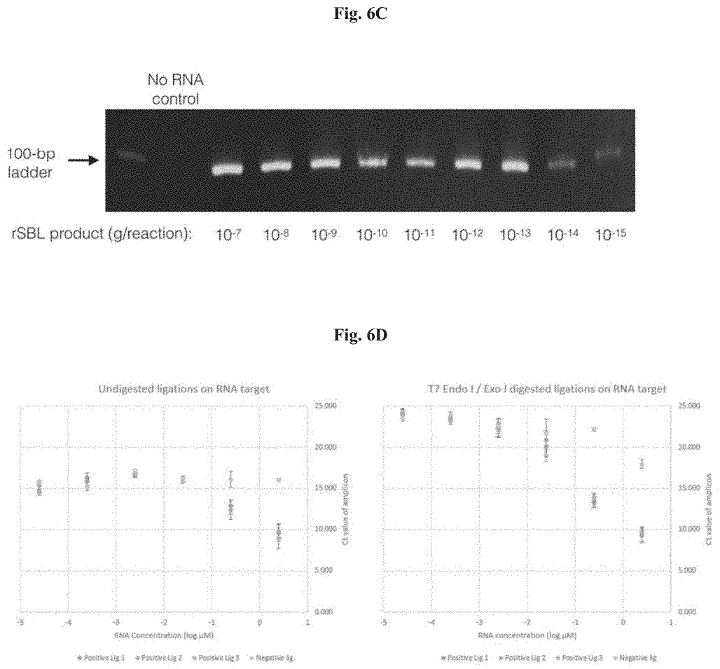

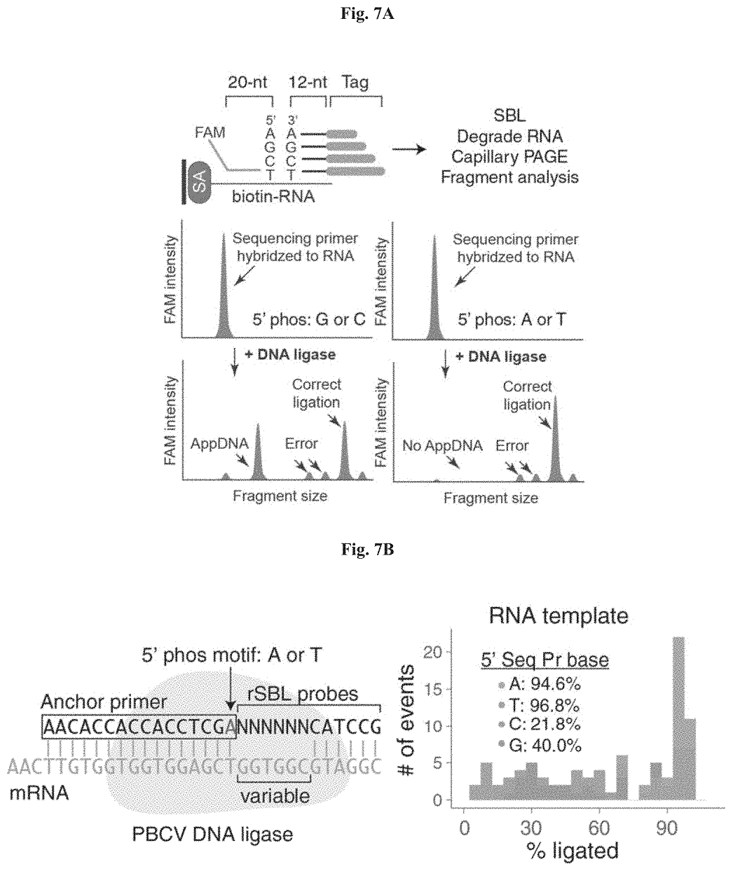

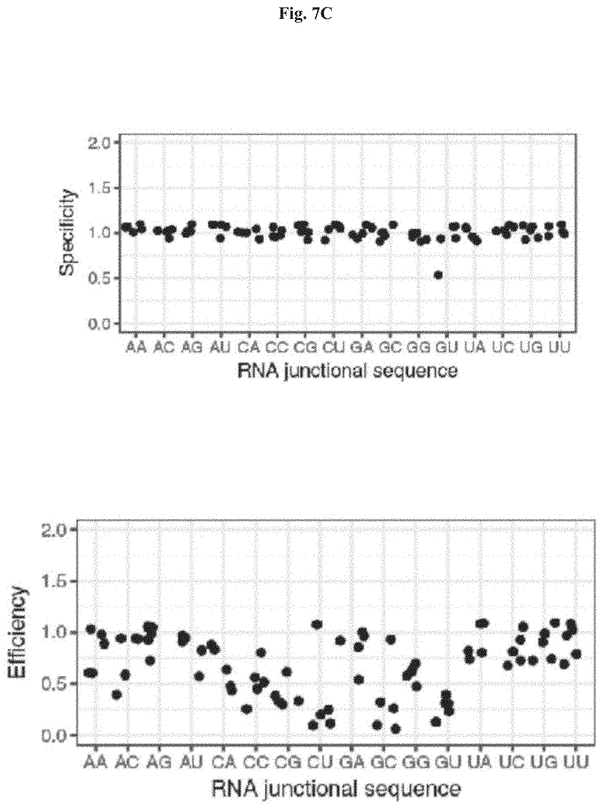

In embodiments of the present invention, an exonuclease degrades sequences not in an SBL product from participating in a PCR reaction. In embodiments of the present invention, the exonuclease is Exo 1, or T7 exo. In embodiments of the present invention, the exonuclease is in combination with an RNAase. In embodiments of the present invention the RNAase is RNASeH or RNase H. (FIG. 6A-6B).

The probe with a variable region L can also be modified using adapter sequences for heterodimer ligation, circularization, and RCA. The adapter sequences can be arranged so that the SBL product is amplified if the subsequence A and B are present. This property can be used to label single cells only when mutation X and Y are both present. Here, adapter sequences for X and Y form a heterodimer concatemer capable of self-circularization and RCA. If either X or Y is missing, the concatemer cannot be formed or circularized.

In further embodiments of the invention the adapter sequences added to the SBL primer and interrogating oligonucleotides can be further modified to include phosphorothioate, locked nucleic acids (LNAs), and other modified bases in order to change their Tₘ or DNA cleavage sensitivity. This is important for ssDNA-specific error correction mechanisms used after SBL, if one were to utilize the adapter sequence for PCR amplification or NGS.

Other embodiments of the invention include an acrydite, azide, or biotin moiety for conditionally immobilizing the SBL product based on the sequence detected. Embodiments may also include a phosphorothioate, inverted T, or inert spacer moiety for conditionally blocking exonuclease digestion. Certain modifications (e.g. deoxyUridine, chimeric RNA nucleotide) can be used to selectively cleave the SBL product, while others (e.g. inverted T, spacers) prevent circular ligation and RCA of specific variant sequences. Together, they can pull-down, isolate, amplify, degrade, or cleave any number of sequence variants or their combinations after SBL for a variety of applications.

Additional embodiments of the inventions include digoxigenin, digoxin, HRP, alkaline phosphatase, or other moieties used for enzyme-linked assays. In embodiments of the present invention SBL may be used to conjugate a specific enzyme activity to a subset of DNA or RNA sequences, followed by the degradation of error-associated or off-target probe-enzyme binding. When used in conjunction with the appropriate enzyme substrate, any specific or general category of DNA or RNA variants can be detected using a fluorescent or colorimetric assay. Such a method could be suitable for rapid and highly multiplexed testing for the presence of mutant cells, pathogens, contaminants, DNA/RNA-based molecular diagnostic markers using a portable, point-of-care device. In embodiments of the present invention the method provides a substitute for antibodies in enzyme-linked assays to estimate the abundance of mutant proteins by quantifying non-synonymous codon alterations directly from the cell or tissue lysate for point-of-care clinical applications.

SBL can be performed using PBCV-1 DNA ligase or similar ligases capable of DNA ligation splinted by RNA (FIG. 21). Instead of one copy of somatic mutations in the genomic DNA, the copy number of somatic mutations expressed as RNA and can be much higher. In embodiments of the present invention the copy number can in the 100s. Recently, PBCV-1 DNA ligase was shown to have a surprisingly strong activity on the RNA template. In embodiments of the present invention the SBL ligation error rate of PBCV DNA ligase on RNA is 2-10%. In embodiments of the present invention the SBL ligation error rate ranges from 1-10%, depending on the base position (FIG. 3A-B). In embodiments of the present invention, at base position +1 and/or +2, from the ligation junction, the base call error rate is ˜2% without any error correction (FIG. 3A). In embodiments of the present invention, at base position +1 from the ligation junction the base call error rate is or less than 1-2% without any error correction.

Embodiments of the present invention providing RNA-based SBL using PBCV DNA ligase using four competing oligonucleotides detect up to 50% of the sequencing primer bound to the RNA template within one minute at 25° C. or 37° C. After 60 min, the sensitivity of RNA SBL is 75% and/or 90%, respectively (FIG. 22A-B). The specificity of base recognition is largely or entirely, invariant of the temperature, salt concentration, or ATP concentration. In embodiments of the present invention the 5′ phosphorylated base of the sequencing primer is critical to ligation efficiency. In embodiments of the present invention the 5′ phosphorylated base is A or T (FIG. 23).

Since RNA-based somatic mutations are present in multiple copies (generally ˜20 or more for common oncogenes), embodiments of the present invention provide SBL reads to call mutations with a low false positive and negative rate even in the presence of a high error rate (e.g. long-read sequencing).

To generate a consensus base call, it is necessary to attach metadata associated with their origin to each read. Accordingly, embodiments of the present invention may include the use of UMIs for individual molecules, for example, when technical noise during molecular amplification may be an issue. To compare all reads from a given cell, embodiments of the present invention may label SBL reads with the cellular ‘UMI.’ For example, individual cells can be sorted into separate wells. In such embodiments, since all SBL reads come from a single cell, they can be averaged to eliminate random sequencing errors and identify true biological variants. Other embodiments localize individual reads in single cells in situ. Therefore, the accuracy of SBL for identifying somatic mutations from a single cell depends on its compatibility with single cell manipulation and analysis.

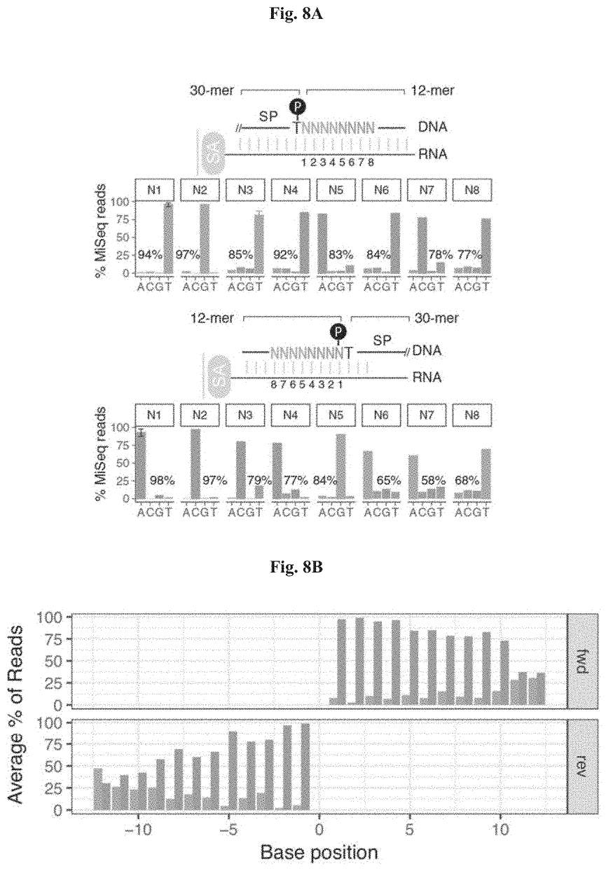

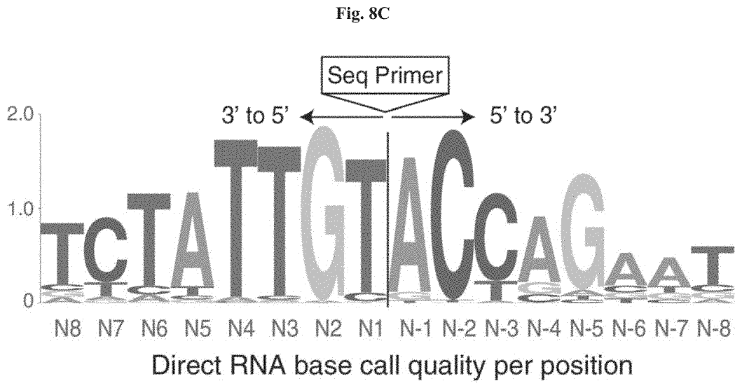

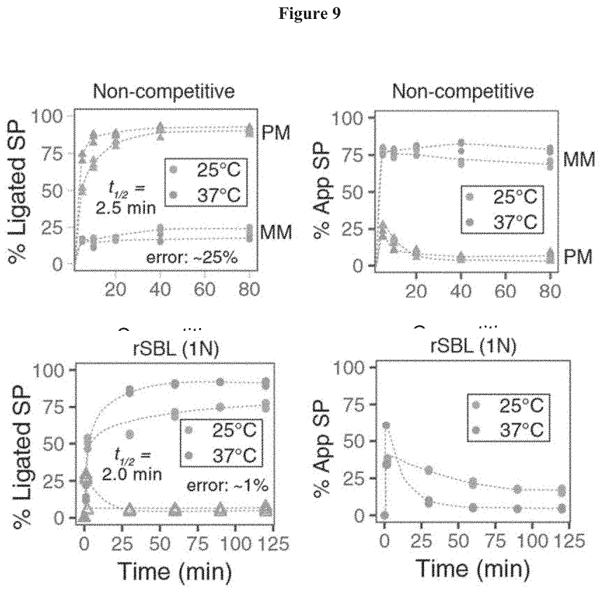

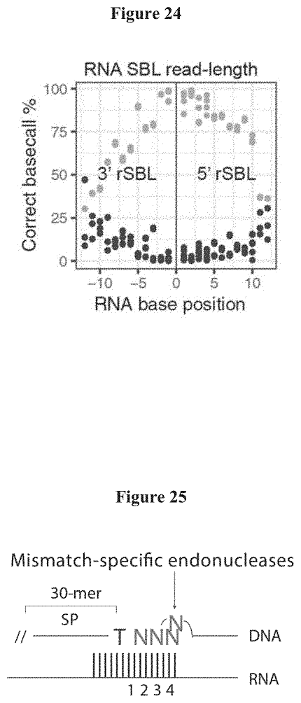

In embodiments of the present invention C or G may be present adjacent to the target of interest, lowering its rSBL efficiency; however, base-specificity extends from the ligation site for up to 3-bases with greater than 90% specificity in both 5′ and 3′ rSBL direction (FIG. 24A), enabling one to shift the sequencing primer by up to 3-bases to avoid C or G at the ligation junction. After base position 3 from the ligation junction, the error rate rises steadily up to 50% past the footprint of PBCV-1 DNA ligase (FIG. 24A); however, errors are random and uniformly distributed across the remaining incorrect bases (FIG. 8A), enabling one to make a base call even past base position 8.

In further embodiments of the invention SBL primers and interrogating k-mer oligonucleotides can include ribonucleotide, inosine, locked nucleic acids (LNAs), and other modified bases in order to change their Tₘ and their probe length in order to maintain the balance of k-mer hybridization and exchange of mismatched oligonucleotides at a given reaction temperature.

Other embodiments of the invention include an acrydite, amino-allyl, azide, or biotin moiety for conditionally immobilizing the SBL product. Embodiments may also include a phosphorothioate, inverted T, or inert spacer moiety for conditionally blocking exonuclease digestion. Together, they can pull-down, isolate, amplify, degrade, or cleave any number of sequence variants or their combinations after rSBL.

In embodiments of the present invention SBL is implemented on DNA or RNA using click chemistry using alkenyl and azide modifications to the sequencing primer and k-mer ends. The reaction condition of click-based DNA ligation is adjusted to maximize the difference of ligation between matched and mismatched k-mer probes. In another embodiment of the present invention rSBL is performed using ribozyme sequences incorporated into either the sequencing primer or k-mers, in which ribozyme sequences are evolved for ligating DNA probes on RNA templates with different kinetics depending on the number of mismatches.

In embodiments of the present invention SBL is implemented using k-mers that are ligated to the sequencing primer by T7 RNA ligase or other ligases capable of joining 5′ and 3′ RNA ends. Embodiments include RNA k-mers that contain tracer RNA, RNA aptamers, ribozymes, or other RNA-based functional groups for programmable activation in vitro.

The result of a successful SBL reaction is a single-stranded DNA product hybridized to each sequenced template. This allows one to selectively label, pull-down, or amplify any subset of sequences after SBL in addition to removing or degrading wild-type sequences that could interfere with the rare variant detection.

If rSBL occurs on the RNA template, it results in the formation of a DNA-RNA duplex. If error in rSBL were to occur, it creates one or more mismatches between DNA and RNA strands. Embodiments of the present invention provide use of an endonucleases, resolvases, or ssDNA-binding proteins which can recognize such mismatches (FIG. 25). This will cleave error-containing rSBL products so that they cannot be labeled, sorted, or amplified, improving the base calling accuracy of RNA-templated SBL (rSBL).

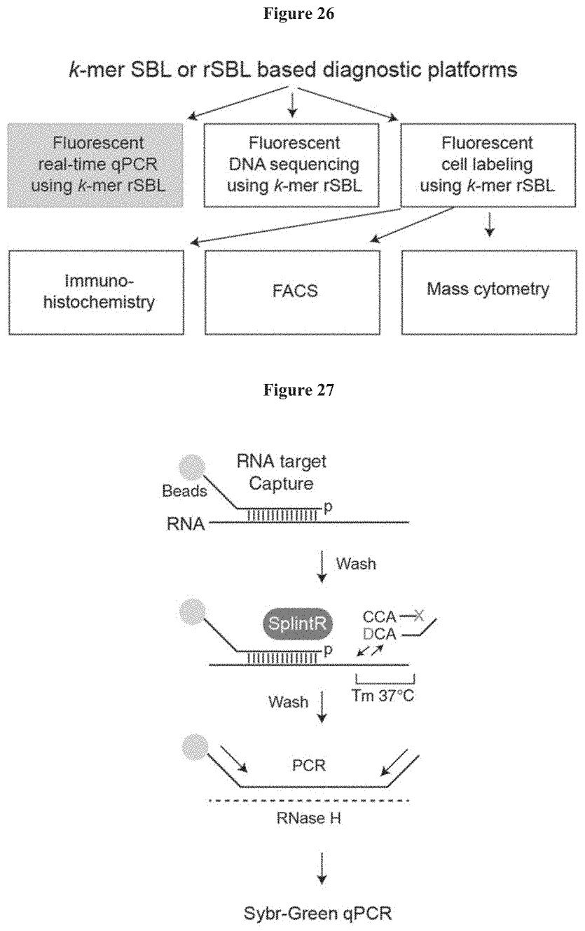

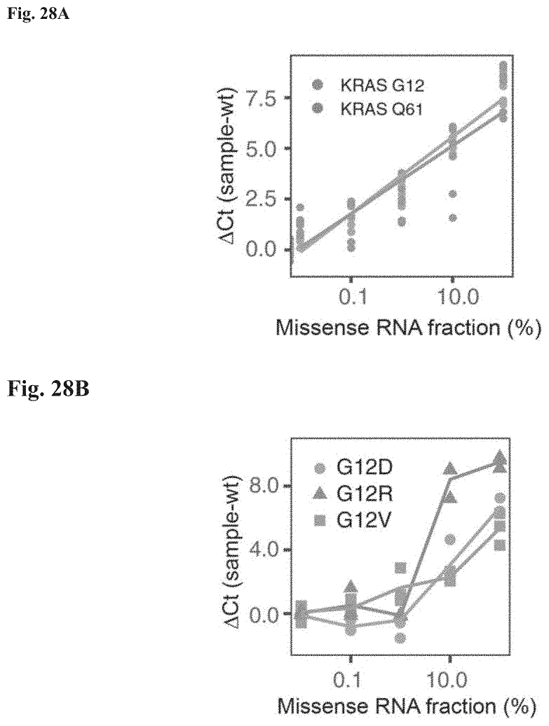

In embodiments of the present invention, exonucleases degrade sequences not incorporated into SBL products and prevent them from being PCR amplified (FIG. 26) for applications utilizing real-time Sybr-Green qPCR (FIG. 27), TaqMan PCR, and digital droplet PCR for quantifying DNA or RNA bearing deleterious mutations of unknown base-composition in order to serve as a sequencing-based but not allele-specific cancer biomarker detection platform (FIG. 28).

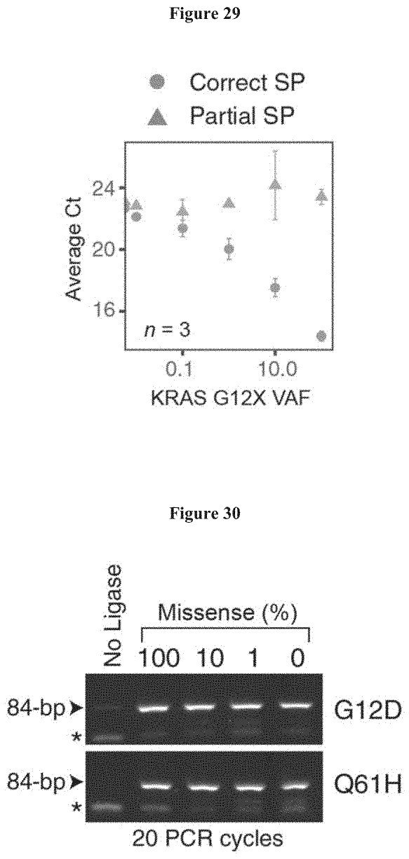

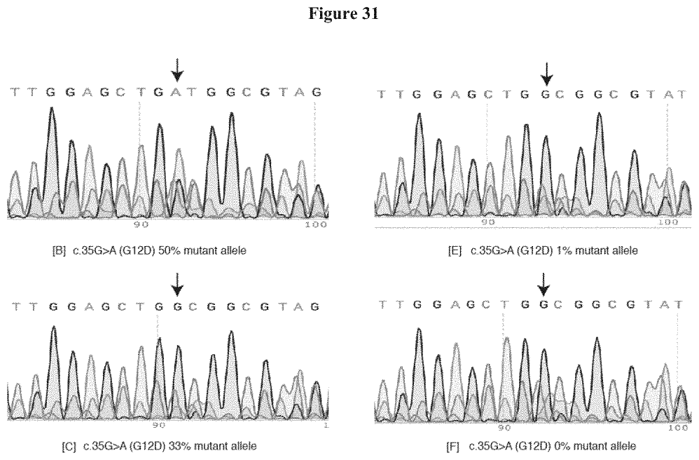

In embodiments of the present invention, target-specific sequencing primers are designed to be orthogonal (FIG. 29) so that up to 100 target-specific sequencing primers can be pooled into one hybridization capture reaction. The PCR amplification step generates DNA fragments of expected sizes (FIG. 30) that can be analyzed by Sanger DNA sequencing (FIG. 31), enabling one to detect rare functional variant DNA or RNA without the need for deep sequencing in one step.

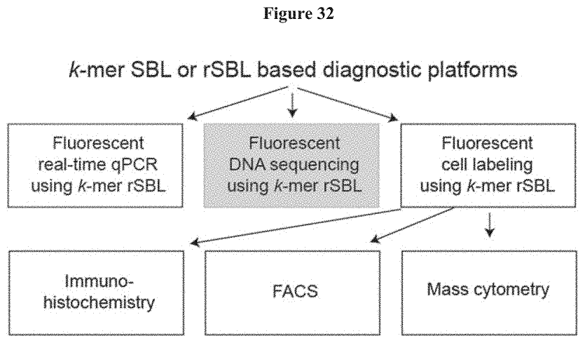

In embodiments of the present invention, absolute rather than relative RNA quantification is possible by performing rSBL directly on single-molecules or molecular amplicons in a flow cell or on glass, similar to Nanostring or Illumina NGS platforms. Embodiments of this invention enable one to quantify or sequence only those molecules bearing deleterious functional mutations, significantly lowering the bandwidth needed to quantify low-abundance nucleic acids associated with early cancer (FIG. 32).

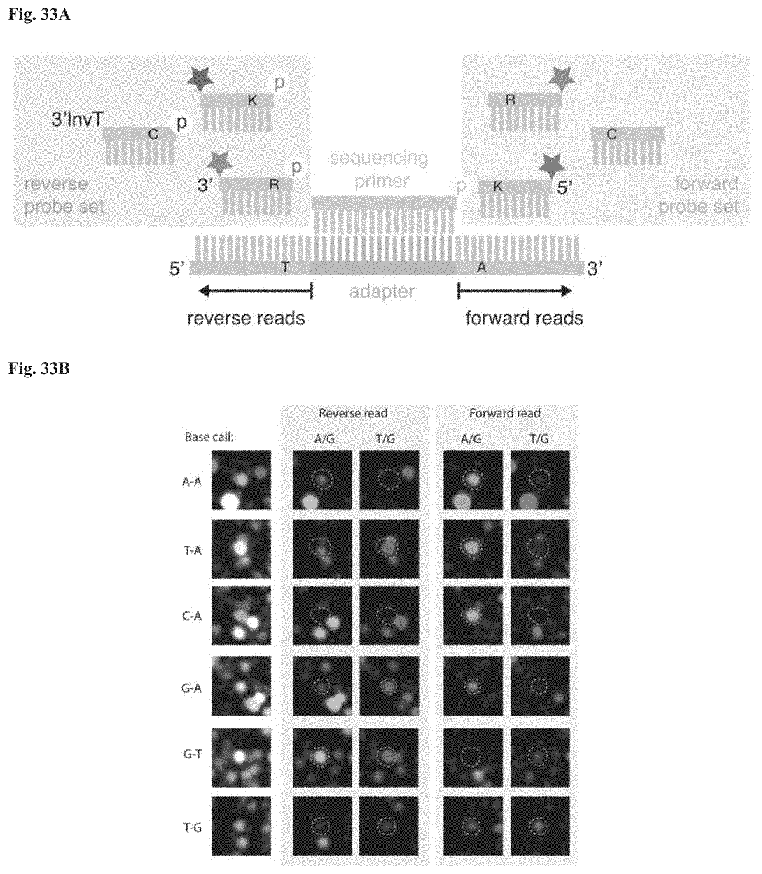

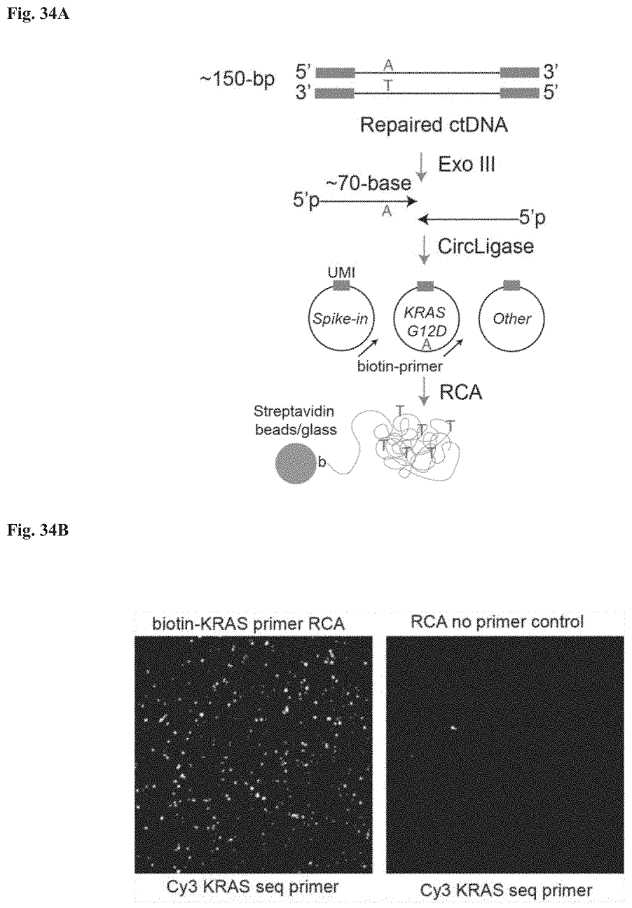

In embodiments of the present invention, k-mers bearing one or more mixed bases (e.g. K or R) are used for rSBL either at 5′ or 3′ ends of the sequencing primer (FIG. 33-A-B). Mixed-nucleotide rSBL is more efficient and robust than fully degenerate (N) k-mers because the sequencing complexity of probe sequences is lower. Embodiments of this invention enables one to convert cell-free DNA into molecular amplicons using in situ PCR or rolling circle amplification (RCA) (FIG. 34A), followed by SBL using k-mers containing a mixed base. k-mers associated with wild-type or non-deleterious mutations are blocked using inverted T opposite from the ligating end. In embodiments of this invention, SBL leads to the formation of extension-ready (e.g. additional cycles of SBL or Sequencing-By-Synthesis) sequencing primers only on those DNA templates containing unknown deleterious mutation (FIG. 34B).

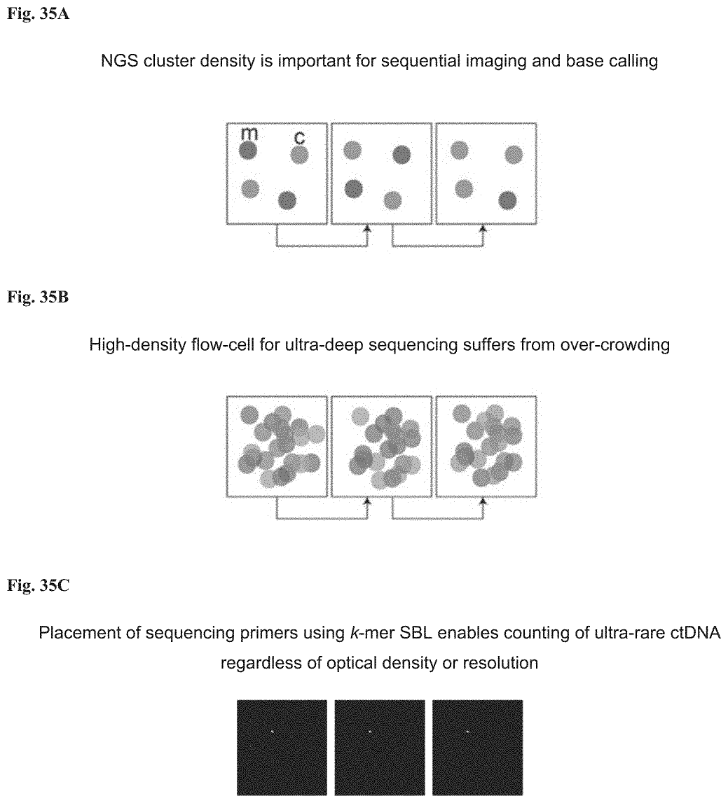

In embodiments of the invention automated fluidics and imaging instrumentation enables quantifying DNA amplicon molecules arrayed on glass or in flow cells using fluorescent single-base extension or NGS (e.g. SBS chemistry); however, only those amplicons containing functionally deleterious variants form productive sequencing primers after k-mer SBL that can be extended and visualized. This embodiment enables one to perform deep-sequencing of billions of single-molecules or molecular amplicons without wasting reads on uninformative sequences (e.g. wild-type, synonymous mutations) (FIG. 35A-C). This embodiment therefore enables one to design small flow cells and instrumentation suitable for low-cost cell-free DNA detection with minimal imaging or reagent cost overhead.

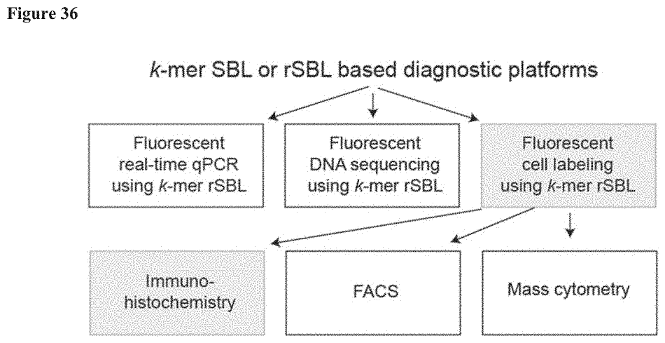

In embodiments of the invention target-specific sequencing primers and k-mers can include universal or barcoded adapter sequences for secondary probe hybridization, in situ PCR, and/or rolling circle amplification (RCA) for detecting rSBL products from programmable k-mers inside chemically fixed cells or tissue sections in situ for cell imaging or in suspension for cell sorting (FIG. 36).

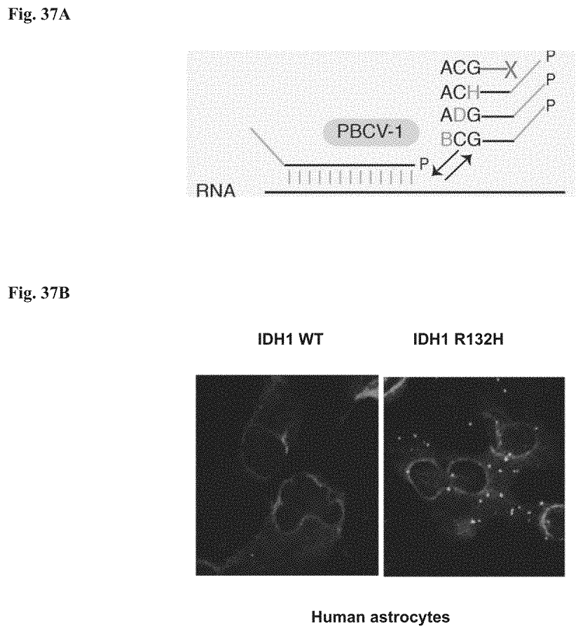

In embodiments of the invention k-mer sequences can differ in their composition by the virtue of containing types of adapter sequences, end modifications, or degradation-resistant phosphate backbone modifications or stem-loop structures for conditional SBL or SBL product degradation or amplification (FIG. 37A), in order to selectively filter out informative, non-functional sequence variants. Embodiments of this invention enables one to detect or label single cells with non-synonymous cancer driver mutations of unknown sequences in situ (FIG. 37B) or in solution.

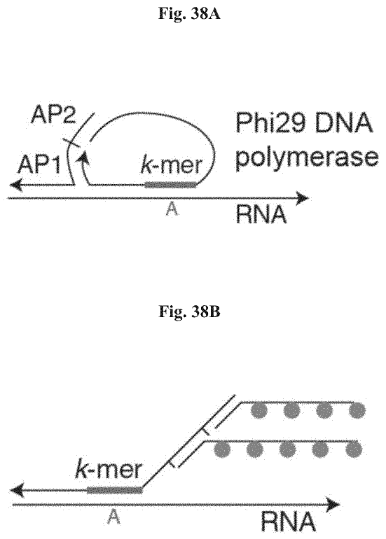

In embodiments of the invention the sequencing primer can incorporate existing methods for detecting DNA probes in situ through molecular amplification (FIG. 38A) or hybridization-based signal amplification (FIG. 38B). Embodiments of this invention can also incorporate enzyme (e.g. CircLigase, T4 DNA ligase)- or chemistry (e.g. Click)-based self-circularization (FIG. 39A) or concatemer formation (FIG. 39B), followed by phi29 DNA polymerase-dependent RCA or multiple displacement amplification (MDA) in situ. In embodiments of this invention that utilizes RCA to generate discrete amplicons followed by fluorescent in situ hybridization (FISH) to common adapter sequences, high magnification microscopy and instrument are used to quantify the number of rSBL products in single cells or tissues (FIG. 39A). In embodiment of this invention that utilizes concatemer formation from rSBL products and in situ MDA (FIG. 39B), the massive signal gain enables one to use lower magnification and low-resolution microscopy for identifying single cells containing functional sequence variants of interest in situ.

In embodiments of the invention rSBL products bound to RNA inside single cells in situ can be amplified 100-1,000-fold using sequential antibody-based amplification (e.g. primary and secondary antibodies), followed by enzymatic conversion of cell labeling substrates (e.g. fluorescein-labeled tyramide) (FIG. 10A). In embodiments of the invention non-specific signal from unincorporated k-mers or un-ligated sequencing primers are reduced by exonuclease-mediated DNA degradation (FIG. 10B). Phosphorothioate modifications are introduced into sequencing primers so that it serves as a blocking group to protect properly ligated k-mers from digestion.

In embodiments of the present invention additional signal amplification is achieved by ligating or hybridizing reporter molecules comprised of short oligonucleotide monomers bearing modifications suitable for fluorescence or colorimetric detection (FIG. 11A). After rSBL, concatemers are assembled on the rSBL product in situ, which enables phosphorothioated sequencing primers to protect properly ligated concatemers from exonuclease-mediated digestion even in the presence of internal DNA modifications for labeling (e.g. digoxigenin) (FIG. 11B).

In embodiments of the present invention T7 or SP6 bacteriophage promoters attached to k-mer interrogation probes can be used to synthesize short RNA transcripts using in vitro transcription (IVT). RNA molecules are functionally modified during or after IVT to reduce its diffusion through cross-linking (e.g. aminoallyl UTP, biotin UTP). Embodiments of the invention containing T7 or SP6 promoters enable one to translate synthetic peptides in vitro or in situ using in vitro transcription and translation systems (e.g. PURExpress from NEB). Such peptides can be short tags (e.g. His 6× tag, Flag tag, HA tag) or longer enzymes or fluorescent proteins (e.g. GFP, RFP). Depending on the number of rSBL products, embodiments of the invention enables multiple signal amplification steps (e.g. in vitro transcription: ˜100-fold, in vitro translation: ˜1000-fold, 1° and 2° antibodies: ˜1000 fold, FITC-tyramide converting enzyme: ˜100-fold), mimicking a massive level of signal amplification that occurs from genomic DNA to proteins inside single cells. After in vitro transcription/translation, cell culture or tissue section slides can be used for standard immunohistochemistry (IHC) using anti-tag primary antibodies.

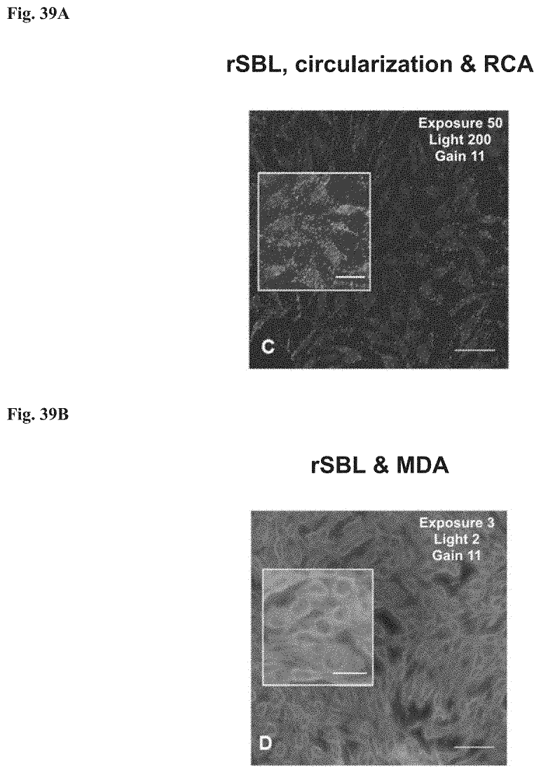

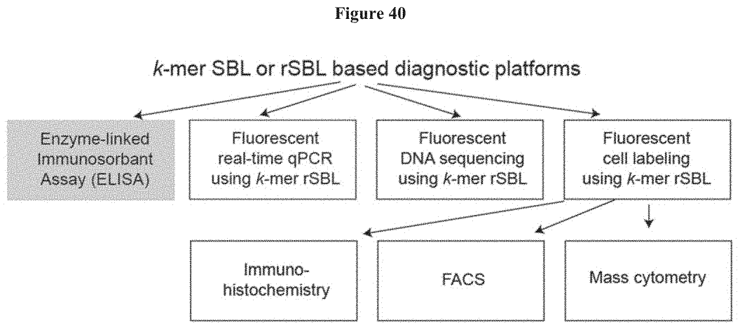

In embodiments of the invention, programmable sequencing of functional mutations using rSBL using partially degenerate k-mers is performed on disposable paper, dip stick, or other forms of solid substrate to ‘fish out’ desired nucleic variants of interest for rapid quantification (FIG. 40).

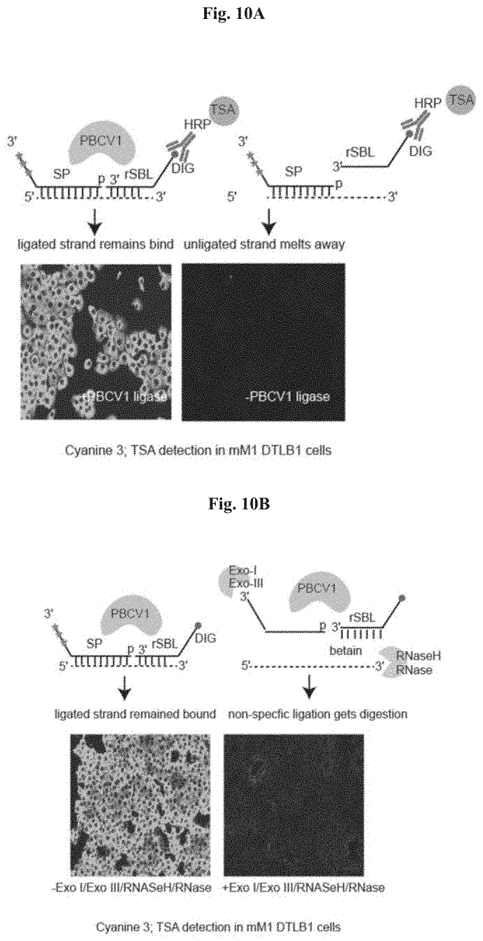

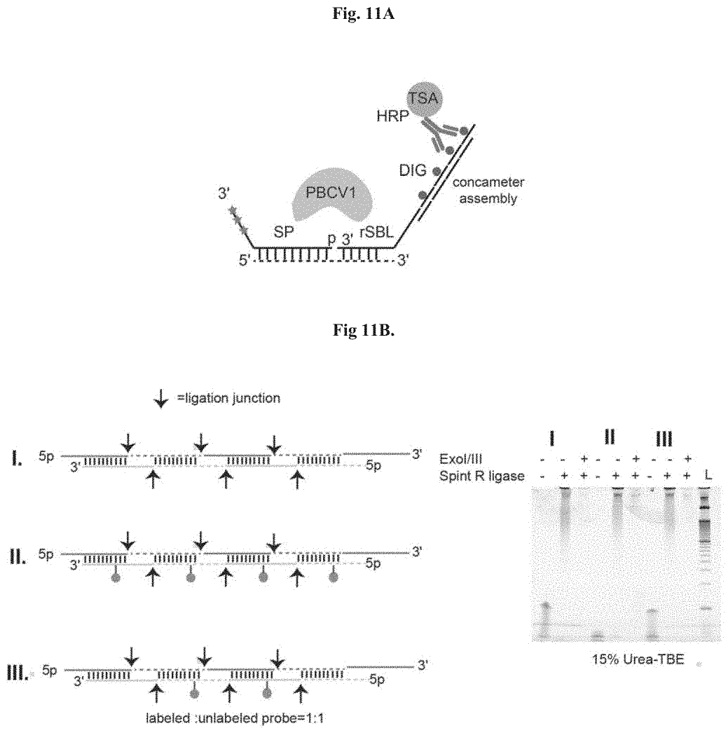

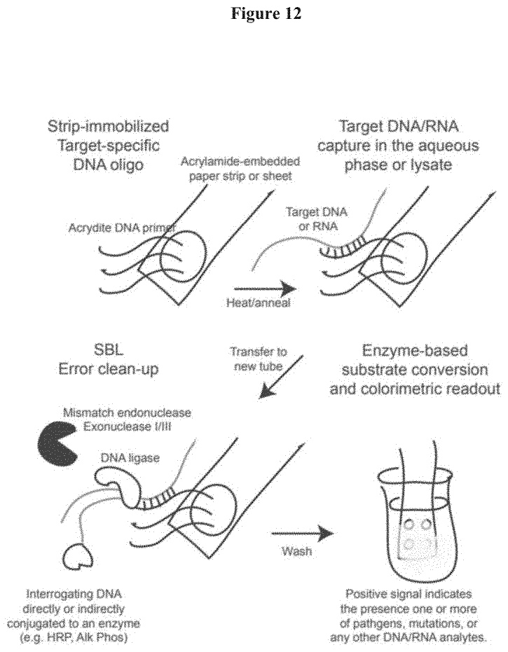

In embodiments of the invention, target-specific sequencing primers are immobilized onto a solid substrate. The paper strip is immersed in the sample (e.g. tissue lysate, concentrated blood, body fluids) to capture desired nucleic acids of interest, followed by a wash cycle to remove excess. The paper strip is transferred to another tube for rSBL with programmable k-mers that possess signal amplification functional groups (e.g. horseradish peroxidase, alkaline phosphatase, digoxigenin, FITC). The paper strip is washed again, and it is then transferred to a signal read-out tube containing enzyme substrates (FIG. 12).

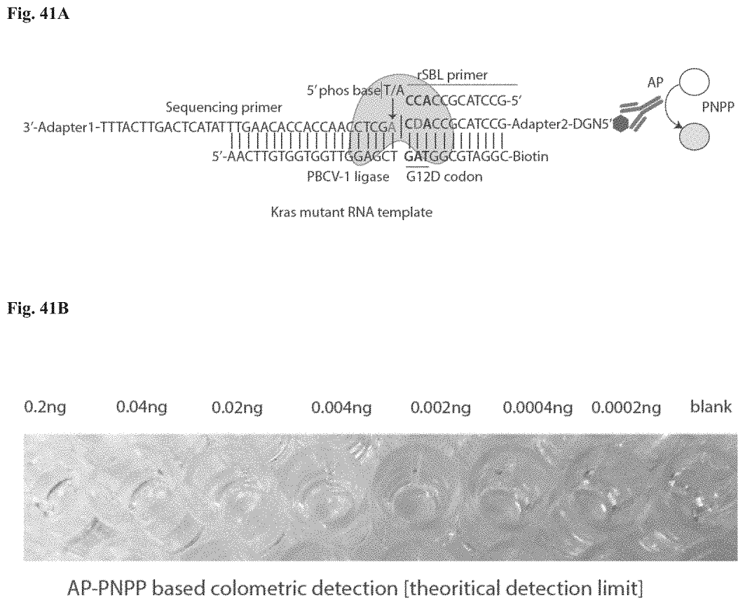

Embodiments of this invention may include driver codon mutation probes against KRAS (FIG. 41A) to detect the presence of functionally deleterious mutations in DNA or RNA from tissue samples, including blood or bodily fluids. By concentrating the specimen, utilizing sensitive signal amplification methods (e.g. antibody-based, branched oligonucleotides), and converting enzymatic substrates for a colorimetric read-out (FIG. 41B), one can determine the presence or absence of contaminating oncogenic mutations in the sample. In embodiments of this invention, enzyme-linked k-mer based SBL of nucleic acids can be used in conjunction with portable devices or instruments for point-of-care assessment of tumor burden or contamination (e.g. during surgical resection to obtain tumor-free margins).

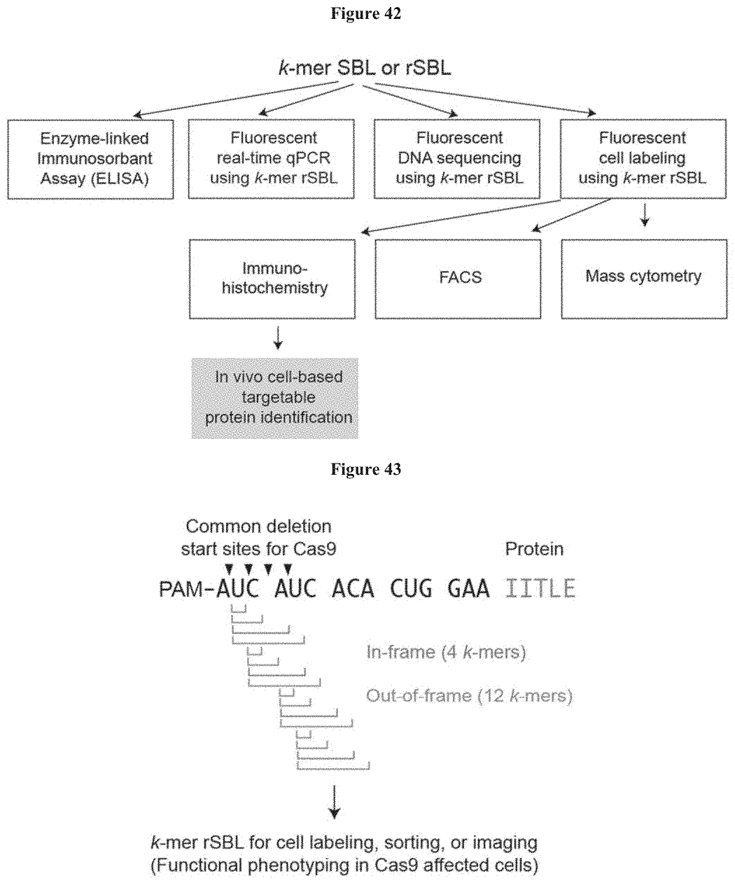

Embodiments of the present invention include the use of loci-specific probe design principles to label single cells using induced somatic mutations, for example through the Cas9/CRISPR system. (FIG. 42). Cas9-induced somatic mutations cause short deletions in their target. The size and location of the deletion are variable. This enables the detection and isolation of cells based on Cas9-targeted loci and its alterations. For example, a protein could be targeted to generate a unique deletion in each cell across the whole protein. Degenerate primers from the present invention may be designed based on the expected change or shift in the target sequence, including in-frame shift mutations (FIG. 43). Each protein-specific panel may be combined with SBL and signal amplification methods to quantify the effect of different protein domains (FIG. 44A) on cellular behavior (FIG. 44B). Embodiments of the present invention delineate the protein domains essential for targeted molecular therapy and drug screening in a massively multiplexed manner, using cellular phenotype assays commonly used (e.g. cell migration, cell invasion, proliferation, cell death, cell transformation). Embodiments of the present invention delineate protein domains with single-cell resolution without relying on traditional NGS or expression of mutated or truncated protein sequences one at a time in vivo.

Embodiments of the present invention read any genetic information of length L in single cells. In specific embodiments, the location of such genetic information that is written or edited can be interspersed throughout the genome, as in cancer point mutations or Cas9-induced insertions or deletions. Embodiments of the present invention convert this information into short single-stranded DNA fragments inside the cell for signal amplification and oligonucleotide detection. The short DNA fragments are stable and amenable to single molecule amplification in solution or in situ. Embodiments of the present invention may assemble the short DNA fragments into larger polymers using specific end-joining adapter sequences. Such polymeric structures from the short DNA fragments derived from SBL can be amplified and interrogated in solution or in situ to generate a consensus read, since the number of polymerizable DNA fragments can be adjusted by varying the number of unique ends for end-joining (In embodiments of the present invention such DNA polymers could come from SBL products from multiple loci, and can be either linear or circular for signal amplification using strand-displacing DNA polymerases (e.g. Phi29).

Embodiments of the present invention utilize barcoded SBL-capable oligonucleotides for readout of individual bases. To discriminate individual SBL products that represent one specific sequence, embodiments of the present invention may sequence every base in single-stranded DNA fragments using molecular sequencing (e.g. SBL) post signal amplification. Additional embodiments barcode individual oligonucleotides in a manner to allow easier discrimination using probe hybridization, antibody-based detection, or any other means of affinity-based detection. For the latter, individual oligonucleotides capable of representing the genetic information in single cells have to be synthesized. For large L, massively parallel synthesis of modified or barcoded oligonucleotides can become rate-limiting (e.g. 4¹²). Embodiments of the present invention can reduce the complexity of interrogation oligonucleotides by a factor of several orders of magnitude (e.g. ˜470,000-fold reduction at L=12 for single point mutations), allowing for affordable synthesis of individual single-stranded DNA probes for sequencing, enabling rapid, non-enzymatic interrogation of individual point mutations, polymorphisms, or variants using probe hybridization to barcode sequences, significantly shortening interrogation time. This can be used to sequence multiple loci for point mutations using ordinary high-throughput oligonucleotide synthesis platforms (e.g. Custom Arrays, LC Sciences, IDT), followed by a probe hybridization-based rapid readout. This enables easier, non-enzymatic ‘painting’ of single cells in a tissue section or cell culture for their single-nucleotide variant profiles, including tumor mutations, akin to general tissue stains used in medical pathology.

In situ hybridization of short probes results in diffuse background or non-specific binding to fixed proteins or nucleic acids due to charge-charge and hydrophobic interactions. In single-molecule FISH, multiple probes co-localize on the same molecule to generate high SNR. Alternatively, molecular inversion probes can generate high SNR only when the two arms of the probe are ligated together. In these methods, false positives are of concern, especially when trying to detect single-base differences with high-sensitivity amplification techniques. In embodiments of the present invention, non-specifically bound probes are completely degraded upon successful SBL on RNA. In these embodiments only fully ligated products are capable of surviving, for example, after exonuclease degradation, and initiate in vitro transcription (IVT) from RNA reporters in situ. In embodiments of the present invention 8 to 12 hour IVT is sufficient to amplify the bound probe and signal amplification can continue indefinitely as long as fresh enzymes are continually added. Because of the absence of non-specific probe binding, SNR increases linearly over time, embodiments of the present invention allow one to detect rare transcripts in an allele-specific manner. In embodiments of the present invention synthesized RNA spreads gradually and eventually fills the whole cell, allowing one to perform single-cell quantification in situ using low magnification objectives or to classify cells using Fluorescence Activated Cell Sorting (FACS) using low-abundance or short transcripts. In further embodiments, reporter RNA can be transcribed from the bound DNA probes even after a protracted archival period or protein immunocytochemistry. To visualize the amplified reporter RNA, fluorescent UTP can be directly incorporated during IVT for one-color assay, or barcoded reporter RNAs can be used for rapid sequential readout using FISH.

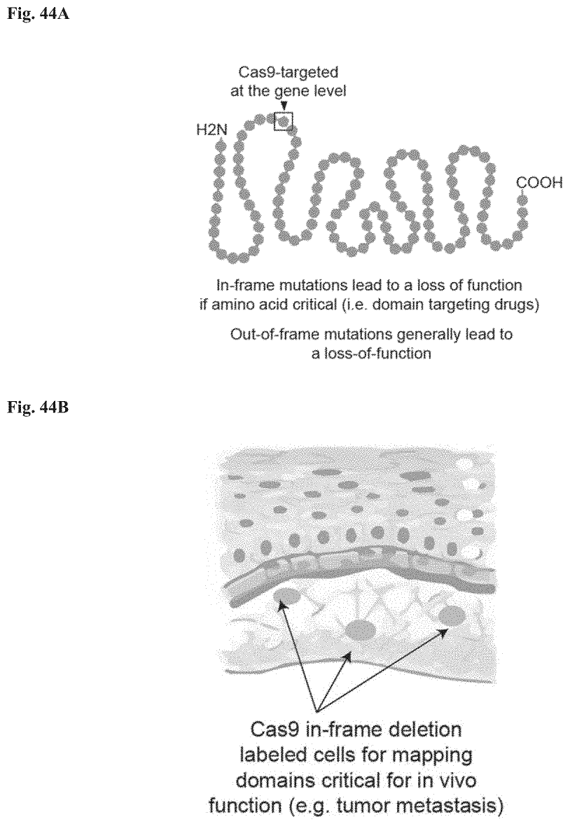

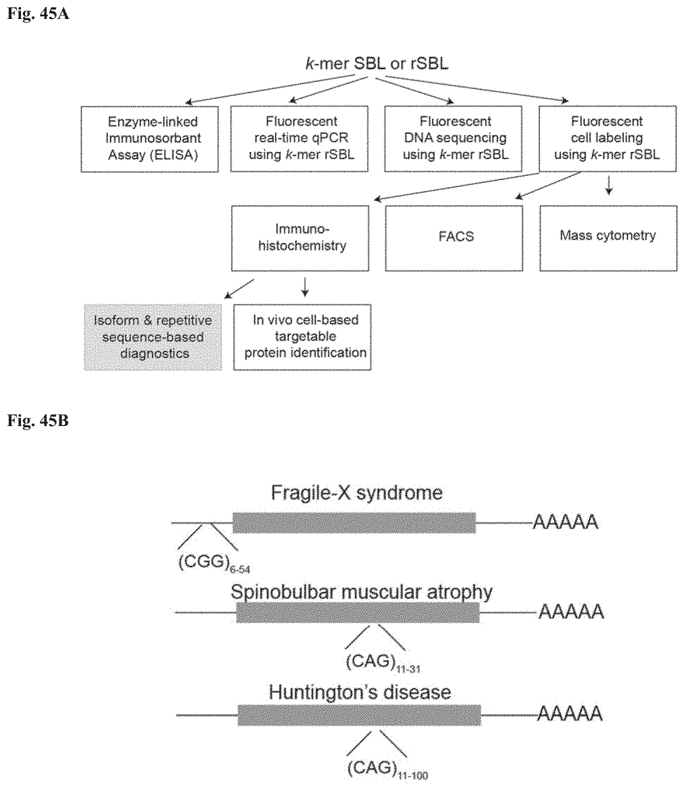

In embodiments of the present invention, programmable k-mers for rSBL are comprised of related sequences that form high repetitive sequences associated with human disease progression (e.g. triplet expansion). Embodiments also include k-mers that bind to small exons and introns that compete for the same splicing acceptor sites (FIG. 45A). Codon expansion in disease-causing proteins (e.g. Huntingtin) is associated with the severity of disease, and embodiments of the present invention enables one to sequentially add k-mers to count the number of codon-repeats directly on expressed RNA inside the fixed cell or tissue (FIG. 45B).

In embodiments of the present invention, sequential rSBL counts the number of short sequence repeats using ligation of partially degenerate repeat k-mers that end with a cleavable terminator. Cleavable terminators prevent simultaneous ligation of multiple k-mers on repetitive sequences, and they may include Endonuclease V-based cleavage of DNA (FIG. 46A). Endonuclease V cuts the DNA 2 or 3 bases away from inosine; therefore, phosphorothioate groups are added to define the cleavage site at position 2. This results in efficient cleavage of the k-mer terminator fragment containing FITC fluorophore (FIG. 46B), preparing the rSBL ligation product for another round of rSBL.

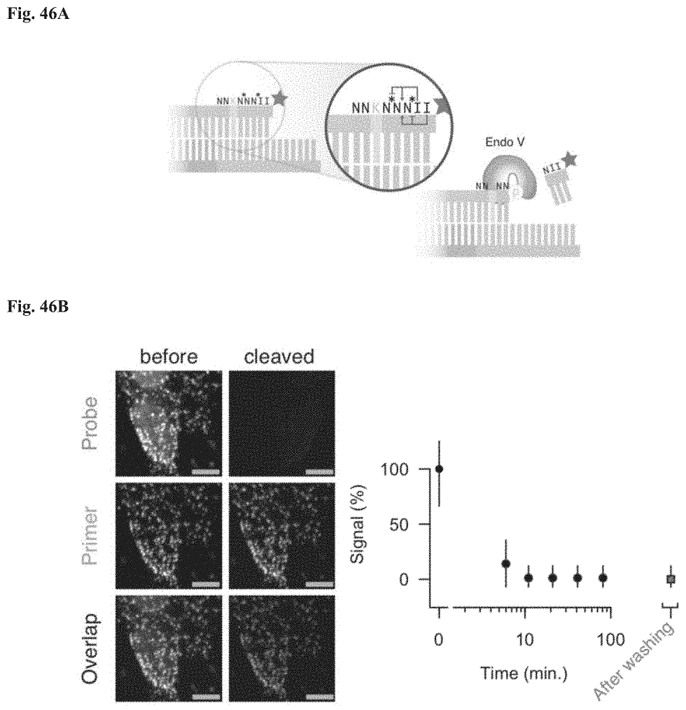

In embodiments of the invention, programmable k-mers are mixed base-containing oligonucleotides that represent a repetitive sequence motif, in which the conserved sequence is a known fixed based while variable bases are represented by mixed-base symbols in the k-mer sequence (FIG. 47A). This enables one to count the number of repetitive sequences regardless of minor variations or polymorphisms. When rSBL reaches the end of the repetitive sequence, ligation cannot proceed. If ligation is quantified by measuring fluorescence from attached fluorophores, the number of ligation cycles prior to the lack of fluorescence marks the number of repetitive sequence expansions (FIG. 47B).

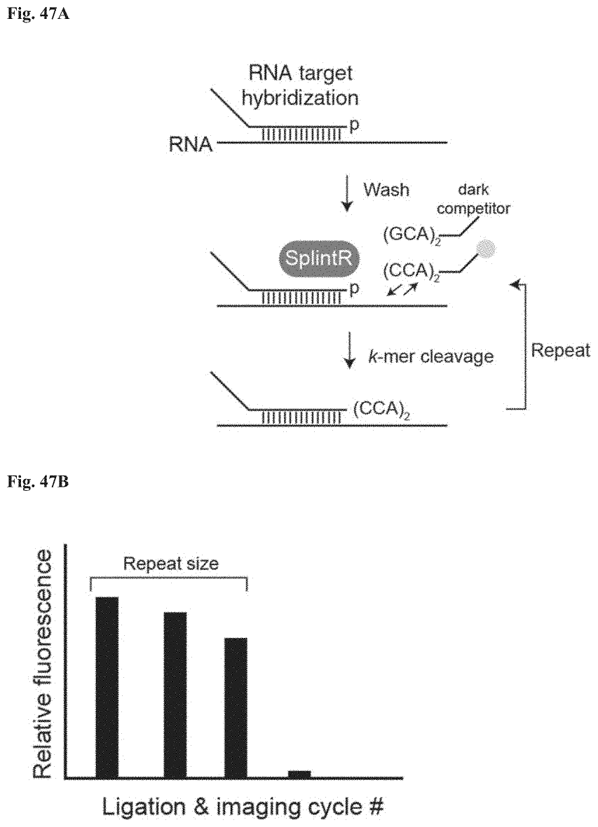

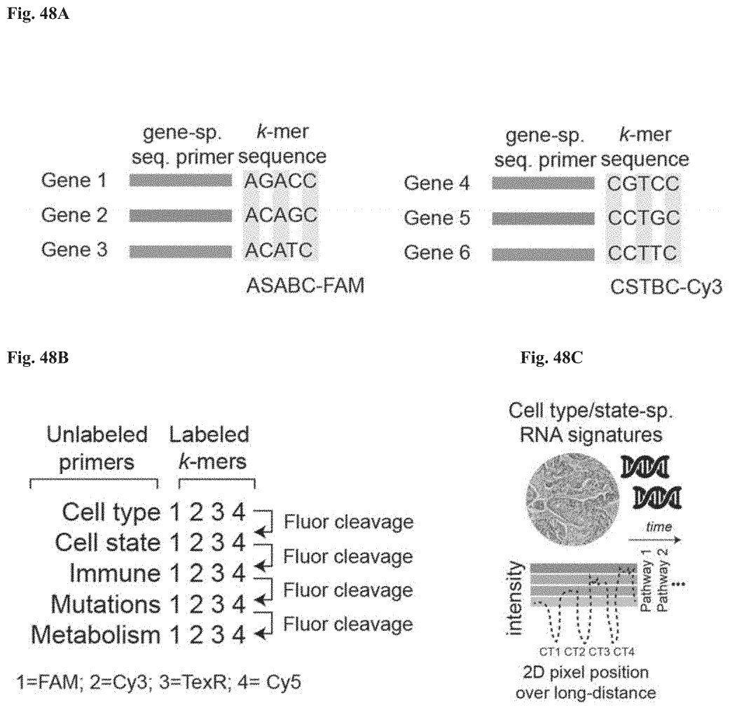

In embodiments of the present invention, programmable k-mers can represent short sequences that are shared by different groups of DNA or RNA molecules (FIG. 48A). Short sequences may be identical, those that share highly similar sequences (e.g. family members), or dissimilar sequences that share a short sequence motif that can be represented using partially degenerate mix-base symbols. Gene or target-specific sequencing primers are hybridized to the sample of interest. Subsequently, k-mers sequences shared by different groups of target sequences downstream of the sequencing primer are ligated using rSBL. Each group of k-mers may represent different functional ontologies or cell states, and each round of rSBL may be followed by cleavage of terminator sequences from k-mers (FIG. 48B). Sequential ligation of k-mers followed by microscopy-based quantification may generate staining patterns characteristic of cell types, signaling processes, or metabolic states based on the presence of relevant nucleic acids that complement standard histological stains (e.g. H&E) or immunohistochemistry (IHC) (FIG. 48C).

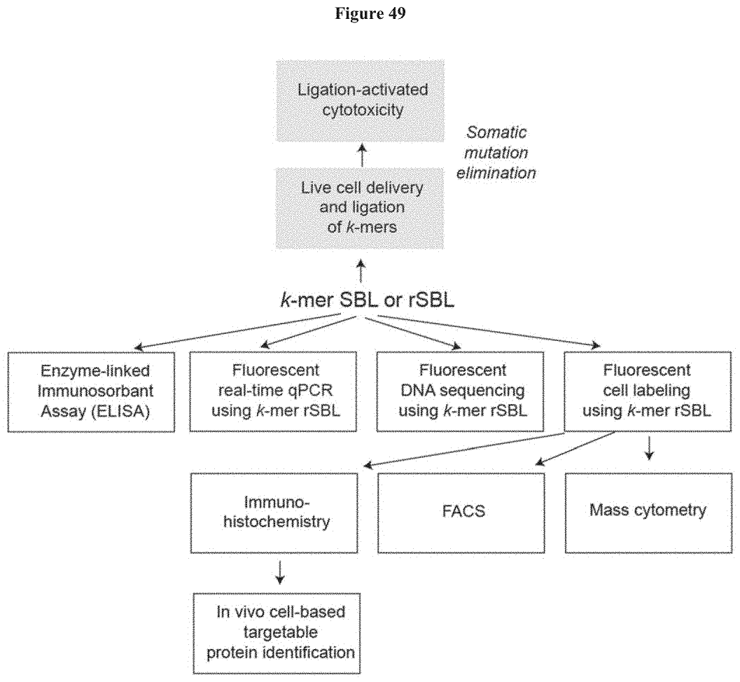

In embodiments of the present invention, rSBL using programmable k-mers may be utilized inside a living cell. Upon successful ligation of rSBL probes to the pathogenic target sequence (e.g. missense or non-sense mutations), in vivo signal amplification is performed to sensitize the cell to external cytotoxic modalities, including pharmacological agents, radiation, viral agents, and immune cells. (FIG. 49).

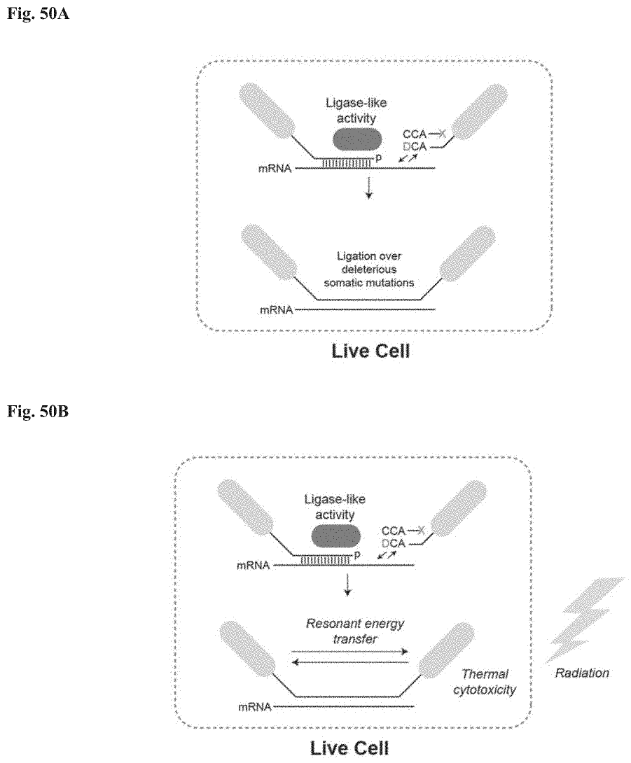

Embodiments of the present invention may use endogenous DNA or RNA ligases, probe-associated ribozymes, or chemical ligation for rSBL in live cells. Anti-sense oligonucleotides that form constituents of the live-cell rSBL mix may include chemical modification to the phosphate backbone of nucleotides for efficient stability and delivery, as long as their effect on Tₘ of k-mers are compensated by changing the probe length of k-mers (FIG. 50A).

In embodiments of the present invention, sequencing primers and k-mers may be covalently attached to functional groups, including metal nanoparticles, split proteins, aptamers, and chemical moiety, that accept and transfer energy from the external source, including microwave and shorter wave radiation, in a proximity-dependent manner. Embodiments of the present invention enables generation of cytotoxic processes (e.g. free radicals, heat, protein modifications, enzyme inhibition) to occur within the cell if a sufficient number of functional nucleic acid mutations are present for k-mer based rSBL (FIG. 50B).

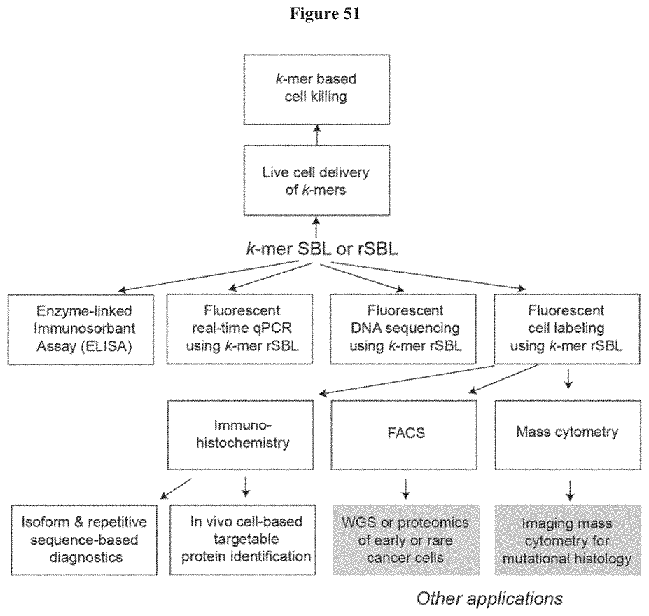

In embodiments of the present invention, rSBL using k-mers may be used to fluorescently label circulating tumor cells based on the presence of functionally deleterious mutations for FACS analysis and subsequent genome or proteome profiling. In other embodiments, rSBL ligation may result from k-mers associated with metal isotopes for mass spectrometry-based imaging or single-cell quantification (FIG. 51).

### Ligases

The PBCV DNA ligase employed in the Examples of this application has been shown to be an effective ligase for the methods described herein. Accordingly, in an embodiment of the invention, the ligase is a DNA ligase that has the same or similar activity as PBCV DNA ligase. Such a ligase can be a homologue of PBCV DNA ligase. For example, the DNA Ligase Encoded by Chlorella Virus PBCV-1 has been characterized in Ho, C. K., et al. (1997), and is found to be suitable for the methods described herein. Furthermore, additional homologues of the PBCV DNA ligase can be readily identified and validated based on the information disclosed herein. Ho, C. K., et al. (1997) in its entirety and/or for the specific description of the Chlorella Virus PBCV-1 DNA Ligase is incorporated herein by reference.

In addition to homologues of PBCV DNA ligase, in another embodiment of the invention, the ligase is produced by rational design, artificial selection and/or directed evolution to have properties analogous to one or more or all of the properties of the PBCV DNA ligase such ligase may, for example, be produced by rational design, artificial selection and/or directed evolution starting, for example, from PBCV DNA ligase or homologues thereof various methods of directed evolution are known in the art (see, e.g. Turner, N. J. (2009)) and can include, for example, directed evolution as described in Arnold, F. H., et al (1999) or computer-aided protein directed evolution as described in Verma, R. et al. (2012j. Turner, N. J. (2009), Arnold, F. H., et al (1999) and Verma, R et al. (2012) in their entirety and/or for the specific description of directed evolution or artificial selection, are incorporated herein by reference.

In another embodiment of the invention, T4 RINA ligase 2 (Rnl2) is found to be effective in the method described herein and is used to ligate the primers and probes in the methods and compositions described herein. Rnl2 has been characterized in Ho, C. K., et al. (2002) and Larman, H. B., et al. (2014) describes detecting of RNA sequences using a modified RNA Annealing, Selection and Ligation (RASL) assay. Ho, C K., et al. (2002) and Larman, H. B., et al. (2014) in their entirety and/or for the specific description of T4 RNA ligase 2 (Rnl2), are incorporated herein by reference. In a further embodiment, the ligase is a homologue of Rnl2.

### Programmable Nucleic Acid Sequencing-by-Ligation for Cell-Free DNA Quantification

In embodiments of the present invention one has a strict control over DNA or RNA molecules that are interrogated in vitro with single-nucleotide specificity. The present invention enables one to amplify, visualize, or sequence functional or clinically relevant nucleic acid variants without the need for specialized target enrichment, targeted library construction, or deep sequencing.

In embodiments of the present invention the entire collection of sampled DNA molecules is amplified but only deleterious mutation-bearing DNA amplicons are sequenced using fluorescently labeled programmable k-mers after rSBL. This enables the operator to overload the sequencing flow-cell with cell-free DNA or their amplicons as molecular over-crowding does not impair imaging. Embodiments may utilize DNA amplicons immobilized onto a flow-cell coupled to optical imaging systems, enabling the detection of ultra-rare circulating tumor DNA molecules in a miniaturized flow cell. Embodiments of this invention may utilize fluorescence imaging of k-mer labeled DNA amplicons, followed by subsequent terminator cleavage and re-ligation for short DNA sequencing using automated fluidics handling.

In embodiments of the present invention the size of DNA amplificons can be made arbitrarily large for high signal-to-noise ratio, since wild-type or non-deleterious molecules do not fluoresce. This enables an instrument to utilize low-cost and low-magnification objectives for quantitative imaging. Such signal amplification methods may include multiple displacement amplification (MDA) of the template DNA. Embodiments of the present invention based on programmable rSBL using k-mers include a portable or benchtop instrument for counting or sequencing ultra-rare cell-free DNA in the blood sample.

In embodiments of the present invention, cell-free DNA detection may be performed by, inter alia: (1) generating short 5′ phosphorylated single-stranded DNA (ssDNA) using exonuclease digestion, asymmetric PCR amplification, or oligonucleotide synthesis, (2) circularization of 5′ phosphorylated ssDNA using end-joining DNA or RNA ligases, (3) binding a 5′ biotinylated RCA primer to a streptavidin or avidin glass or bead to saturation. In preferred embodiments of the present invention the bead is Dynabeads, (4) hybridizing the circularized ssDNA to the bead. (5) adding a DNA polymerase to generate rolling circle amplification products (RCPs). In embodiments of the present invention the polymerase is Phi29 DNA polymerase.

In such embodiments rSBL may be performed by (1) hybridizing an rSBL sequencing primer to RCPs on a bead. In embodiments of the present invention the hybridizing is conducted for 10 minutes. (2) Adding DNA ligase and a fluorescently labeled k-mer. In embodiments of the present invention the reaction is conducted for 60 minutes. In embodiments of the present invention the ligase is T4 DNA ligase. (3) washing un-ligated k-mers from the beads. (4) imaging fluorescently labeled DNA amplicons. In embodiments of the invention the preferred imaging modality is inverted epifluorescence microscopy with a 4-megapixel camera CCD camera.

The following examples are presented in order to more fully illustrate some embodiments of the invention. They should, in no way be construed, however, as limiting the broad scope of the invention.

### EXAMPLES OF THE INVENTIONS

### Example 1: PBCV DNA Ligase Sensitivity

PBCV DNA ligase had originally been described as being incapable of performing DNA-to-DNA ligation when splinted by an RNA template; however, Lohman et al. showed that the enzyme activity is ˜100-times more efficient compared to T4 DNA ligase (Lohman, G. J., et al. (2013)). Others have shown that the single-base specificity is variable, making PBCV DNA ligase ill-suited for high-fidelity RNA sequencing applications. In this example, it was reasoned that the specificity of mismatch recognition in sequencing-by-ligation using T4 DNA ligase comes from the rapid exchange of base-interrogating probes in competition, in addition to the difference in enzymatic kinetics between perfectly matched vs. mismatched base pairs (FIG. 5A-B).

In this example it is tested whether the single-base detection sensitivity and specificity of PBCV DNA ligase could be improved by establishing a solid phase-based in vitro assay. A biotinylated RNA template (30-mer) is bound to Streptavidin beads, a sequencing primer (20-mer) is hybridized to the template in three-fold excess, and the base-interrogating oligonucleotides are added along with PBCV DNA ligase for rSBL using RNA as the template. Because PBCV DNA ligase exhibits nucleotide-specific bias at the ligation junction, all sixteen possible two-base combinations are tested in vitro. Results are quantified using high-throughput capillary gel electrophoresis in order to quantify the absolute amount of the ligated products in addition to any ligation intermediates Single-base discrimination specificity is found to be 100% at position +1 across multiple experiments and probe designs. At room temperature, the specificity is less than perfect for several base combinations, likely due to slower competitive probe exchanges. Deep sequencing of the ligation product demonstrated single-nucleotide specificity ranging from 99% to 99.99% (position +1 to +4) and lower than 99% after base position +8. The ligation efficiency is more variable, but it was >93% as long as the 5′ base of the sequencing primer is either A or T, significantly higher than the allele detection rate of RT-based sequencing methods. Our results here defined the core sequence requirement and the read length for designing sequencing primers and interrogation probes.

### Example 2: PBCV DNA Ligase Temperature Dependence

In this example, immobilized RNA targets in fixed cells is primed using high excess DNA target primer a Hyb buffer (HB) of 10 mM Trist-HCl, 50 mM KCl, and 1.5 mm MgCl₂ at a pH of 7.5-8.0 @b, 25° C. rSBL is conducted for 60 minutes at 37° C. with an rSBL mix of interrogation probes, SplintR NDA ligase, 10 mM Tris-Hcl, 50 mM KCl, and 1.5 mM MgCl₂ at a pH of 7.5-8.0, with 1 mM ATP and 200 μM dNTP. Clean up solution of RNase H, Exo 1, 10 mM Trist-HCl, 50 mM KCl, 1.5 mM MgCl₂ at a pH of 25° C. is added to degrade un-ligated degenerate sequences for 15 minutes at 37° C., and then heated to 95° C. for 5 minutes. PCR is conducted for 30× cycles using a PCR primer solution of Hot start Taq, PCR primers, dNTP, 10 mM Trist-HCl, 50 mM KCl, and 1.5 mM MgCl₂ at a pH of 25° C.

PBCV DNA ligase requires >8-bases for >90% ligation efficiency. Longer N-mers (>12-bases) do not compete well at 25° C. due to higher Tₘ and lead to misincorporation and base errors (>5% vs. 1-2%). PBCV DNA ligase works at 25° C. or 37° C. The ideal reaction temperature is 37° C., and the ideal N-mer length is 12 to obtain requisite sensitivity and specificity.

Increased error rate is found at 25° C. versus 37° C., with ligation efficiency significantly lower at 8-mer ligation. 5′ inverted dT was required to block degradation of correctly ligated product.

### Example 3: Mutation Detection in Suspended Cells

In this example, the exceptional sensitivity, specificity, and SNR is applied to detect specific mutations in suspended single cells. The goal is to detect rare tumor cells and to enable volume-filling signal amplification for monitoring or cell sorting for downstream analysis. To assess the sensitivity and the specificity of such an approach, two populations of HEK293 cells expressing CFP or GFP that differ by a single point mutation are mixed. A probe pair to discriminate GFP from CFP mRNA is designed, followed by conditional IVT amplification during which Cy5-UTP used to label the amplified reporter RNA. Fluorescence microscopy to quantify the false negative and positive rate is used, demonstrating unparalleled performance in identifying cells based on a single-nucleotide mutation). To demonstrate the ease-of-use and potential applications, ˜10 GFP-positive cells per million un-labelled cells is spotted on a piece of nitrocellulose. After gel encapsulation, the nitrocellulose strip is dipped across three different tubes (ligation, exonuclease, and IVT). Using basic epifluorescence microscopy, at least one or more GFP-positive cells out of >1-million cells can be detected in ten independent experiments with a false negative and positive rate of <10′. If significant variations were to exist in GFP protein synthesis, the actual false positive rate is even lower. To see whether the transcriptome remained intact after in situ mutation detection, Cy5-positive cells (GFP mRNA) are sorted and standard mRNA-seq is performed before or after in situ RNA mutation detection. Using two million reads per sample, the total number of unique genes was similar (10,000 vs. 12,000), and their global gene expression is highly correlated (R²=0.88; Spemann's p-value <10′).

### Example 4: Short Sequencing of Bar-Codes

To see if our platform can also be used for sequencing short RNA barcodes in situ, a cell line stably expressing GFP and Cas9 is used. After transfecting GFP-specific sgRNA-expressing plasmids, the region downstream of the PAM sequence predicted to contain short somatic indels is interrogated using partly degenerate interrogation probes that are barcoded for each base (+1 to +4). Prior to sgRNA transfection, 99% of the cells were GFP-positive, and 98% of the cells displayed the same GFP template sequence. After sgRNA transfection, however, 52% of the cells lose their GFP fluorescence after 24 hours, each cell displays unique indel sequences in situ as 1-, 2-, or 4-cell mosaics. Of those cells they stayed GFP-positive, 17% now display unique GFP indel sequences, suggesting in-frame indels. Because somatic mutations are sequenced in situ, multiple independent reporters can be introduced and maintain their phase for each cell. In combination with high sensitivity and speed, it possible to interrogate dozens of induced somatic mutations sequentially for comprehensively reconstruct cell lineage or activity information in situ.

### Example 5: Scalability to Heterozygous Mouse Brain

The ultimate goal of cell atlas projects is to scale these approaches for whole tissue, whole organ, or whole organism reconstruction based on molecular or cellular information. To demonstrate the scalability of our platform, 200 adult brain sections are generated from heterozygous mice. Probe pairs for those transcripts most associated with cell identity information from published single-cell RNA studies are generated (See FIG. 14). Each pool of cell type-specific probes is barcoded across eight different cell types and up to 12 genes per cell type After in situ rSBL, exonuclease digestion, and IVT (one day), rapid sequential FISH and whole brain imaging is performed across eight different cell types across 200 tissue sections (12 hrs) with one additional to process, register, visualize, and annotate the image data. Preliminary cell type-specific probe sets required further optimization, however specific cell types are able to be localized in the correct brain regions, along with cell morphologies consistent with the known cell type. In parallel, allele-specific probe sets covering the X chromosome are designed. In the brain of female mice, it is observed mostly a random mosaic of XCI of differing clonal patch sizes) In the hypothalamus and subventricular regions in the prefrontal cortex, the XCI clonal patch sizes are significantly less random, suggesting clonal proliferation and limited migration of precursor cells after neurogenesis. Both cell type and allele-specific RNA detection, imaging, and whole brain reconstruction take less than three days total, generating quantitative single-cell resolution data. Therefore, the platform can be used for hierarchical ‘painting’ of many different types of RNA variants (e.g. RNA splicing, RNA editing, small RNAs) for rapid whole tissue reconstruction to investigate somatic mosaicism in gene regulation.

### Example 6: Fluorescent Probe Hybridization or DNA Barcode Sequencing

In one experimental realization of our method, tissues are dissociated using enzymatic digestion, or the blood is collected and spun down at 4° C. Suspended cells are then fixed in formalin, ethanol, or methanol for 15 min, followed by cell permeabilization, if necessary, using Triton X-100. The sequencing primer is hybridized in situ at 42° C. for 2 hours in the presence of formamide and RNase inhibitors. The excess primer is then washed out, followed by the addition of mutation-scanning probes along with the DNA ligase of choice (e.g. PBCV DNA ligase) for up to 1 hour. Cells are then washed and used for in situ PCR or RCA. For PCR, a pair of 5′ modified primers are used so that one PCR strand can be digested after the PCR reaction. For RCA, the rSBL product is circularized using DNA splinted ligation or CircLigase, followed by strand-displacement amplification using Phi29 DNA. These steps enable fluorescent probe hybridization or DNA barcode sequencing to interrogate individual bases in the amplified product.

### Example 7: Single-Cell Heterogeneity in Somatic Mutations or Allele-Specific Gene Expression

In another experimental realization, single cells are sorted into 96-well plates manually or using FACS into a cell lysis buffer. The sequencing primer is annealed to endogenous mRNA for 2 hour, and mutation scanning probes along with DNA ligase are then added into each well for 1 hour at 37° C. Un-ligated rSBL probes are digested using exonucleases (1, III, or lambda), followed by the heat inactivation of exonucleases. Real-time quantitative PCR is performed using mutation or sequence variant-specific PCR primers, using ΔCt from the wild-type sequence to quantify the relative amounts of mutant alleles on RNA. This method can quantify the single-cell heterogeneity in somatic mutations or allele-specific gene expression. In contrast, current methods for single-cell mutation sequencing or amplification suffer from a high drop-out rate due to their low sensitivity (e.g. inefficiencies in reverse transcription), limiting a quantitative analysis of mutation- or allele-specific gene expression in single cells to highly expressed genes.

### Example 8: In Situ Mapping of Tumor Mutational Heterogeneity

In another experimental realization of our method, fixed frozen OCT-embedded tissues or FFPE tissues are mounted on a glass slide, followed by cell permeabilization using proteinase K. The sequencing primer is hybridized to tissues in situ at 42° C. for 2 to overnight in the presence of formamide and RNase inhibitors. The excess primers are then washed out, followed by the addition of mutation-scanning probes along with DNA ligase of choice (e.g. PBCV DNA ligase) for up to 1 hour. Tissues are then washed and used for in situ PCR or RCA. For PCR, a pair of 5′ modified primers are used so that one PCR strand can be digested after the PCR reaction. For RCA, the rSBL product is circularized using DNA splinted ligation or CircLigase, followed by strand-displacement amplification using Phi29 DNA. These steps enable fluorescent probe hybridization or DNA sequencing (e.g. rSBL) to interrogate individual bases in the amplified product. This allows one to sequence somatic mutations in situ to map the tumor mutational heterogeneity, including other types of RNA variants (e.g. T-cell receptor variants, splicing variants, RNA modifications) spatially.

### Example 9: rSBL Probe Design Steps for a Human KRAS G12 Codon Point Mutation (Designing Programmable rSBL Probes)

Step 1. A first A or T base upstream from a codon-of-interest is identified. If A or T is within 9 bases from the codon, the codon is suitable for sequencing, as indicated below with the codon-of-interest indicated in uppercase and the A or T, here a T, indicated in underline.

For RNA-based SBL, a first A or T base upstream from a codon-of-interest is identified. If A or T is within 6 bases from the codon, the codon is suitable for sequencing, as indicated below with the codon-of-interest indicated in uppercase and the A or T, here a T, indicated in underline. For DNA-based programmable DNA, any base adjacent to the codon sequence is suitable for the targeted primer design.

(SEQ ID NO. 1)
5′ auaaacuugugguguggagcuGGUggcguaggc 3′
(RNA template)

Step 2. A 20-base sequence, or 20- to 35 base sequence (Tₘ˜60-80° C.), going away from the codon sequence is chosen, starting from the chosen A or T base (rSBL) or any adjacent base (SBL), as indicated below in italics. Its reverse complement sequence is generated as the target-specific rSBL primer (bottom strand in the figure below).

(SEQ ID NO: 1)
5′ auaaacuugugguguggagcuGGUggcguaggc 3′
(RNA template)
(SEQ ID NO: 2)
3′ atttgaacaccacacctcga 5′
(rSBL primer)

Step 3. The rSBL primer is 5′ phosphorylated for ligation.

(SEQ ID NO. 2)
3′ atttgaacaccacacctcga/5phos/
(rSBL primer)

The 5′ end of the sequencing primer forming a base-pair with A or T is the rSBL junction, indicated below with a vertical line. The 5′ phosphorylation is not shown for the clarity of presentation. The 5′ end of the sequencing primer forming a base-pair with the DNA template base is the rSBL junction. Only the rSBL junction example is shown below. The hybridization region of the sequencing primer is shown in italics, and the ligation junction is shown as a vertical line through the RNA template sequence.

(SEQ ID NO: 1)
5′ auaaacuugugguguggagcu|GGUggcguaggc 3′
(RNA template)

Step 4. Starting from the ligation junction, 12-bases containing the codon sequence, indicated below in bold are selected. Then its reverse complement sequence is generated.

(SEQ ID NO: 1)
5′ auaaacuugugguguggagcu|GGUggcguaggc 3′
(RNA template)
(SEQ ID NO: 3)
3′ atttgaacaccacacctcga|CCAccgcatccg 5′
(expected wild-type rSBL product)

Step 6. To exclude the wild-type sequence from detection, the sequence complementary to the wild-type codon base is replaced using the mixed bases identified in Table 1.

(SEQ ID NO: 1)
5′ auaaacuugugguguggagcu|GGUggcguaggc 3′
(RNA template)
(SEQ ID NO: 4)
3′ atttgaacaccacacctcga|DDBccgcatccg 5′
(non-wild type complementary sequence)

Step 7. For point mutations, the wild-type complementary sequence is fixed at the other two positions for every mixed base in programmable rSBL probes. The anti-codon sequence is underlined. Note the direction of rSBL probes (5′ to 3′).

(SEQ ID NO: 5
5′ gcc acgccBcc 3′
(SEQ ID NO: 6)
5′ gcctacgccaDc 3′
(SEQ ID NO: 7)
5′ gcctacgccacD 3′

Step 8. To further reduce the probe complexity to non-synonymous mutations, only probes interrogating bases expected to change amino acid identity are used, as, e.g., identifiable from Table 2:

For example, in the case of KRAS G12, the third codon base does not alter amino acid identity. Therefore, only two probes with mixed degeneracy at codon base 2 or 3 are used to detect non-synonymous point mutations from KRAS G12.

(SEQ ID NO: 6)
5′ gcctacgccaDc 3′
(SEQ ID NO: 7)
5′ gcctacgcacD 3′

Step 8. Programmable rSBL probe sequences are added to amplification-enabling primer sequences for PCR, FISH, RCA, or other universal primer-based amplification methods. An example of adapter sequence for PCR or RCA is shown below. Note that the RNA template direction is 5′ to 3′, while the adapter-containing rSBL probe is 3′ to 5′. Adapter 1 is added to the sequencing primer, and Adapter 2 is added to the rSBL probe.

(SEQ ID NO: 1)
5′ auaaacuugugguguggagcu|GGUggcguaggc 3′
(SEQ ID NOs: 2 and 212)
3′ (adapter1)-atttgaacaccacacctcga|
Dcaccgcatccg-(adapter2) 5′
(SEQ ID NOs: 2 and 213)
3′ (adapter1)-atttgaacaccacacctcga|
cDaccgcatccg (adapter2) 5′

Step 9. The wild type rSBL probe is tagged with a scrambled control sequence to block PCR amplification from wild type sequences.

(SEQ ID NO: 1)
5′ auaaacuugugguguggagcu|GGUggcguaggc 3′
(SEQ ID NO: 4)
3′ (adapter1)-atttgaacaccacacctcga|
CCAccgcatccg (Scrambled adapter) 5′

Step 10. This process yields three rSBL interrogation oligonucleotide sequences that are added to amplification or control adapter sequences.

(SEQ ID NO: 6)
5′ (adapter2)-gcctacgccaDc 3′
(SEQ ID NO: 7)
5′ (adapter2)-gcctacgccacD 3′
(SEQ ID NO: 8)
5′ (scrambled adapter)-gcctacgccacc 3′

Step 11. To eliminate excess SBL probes from interfering with signal amplification, phosphothioate (PPT) or inverted T is added to the 3′ end of Adapter 1 in the sequencing primer. This prevents successfully ligated rSBL products from being digested by 3′ exonucleases (e.g. Exo I or III), while un-ligated SBL probes containing (adapter2) sequences are degraded.

(SEQ ID NO: 2)
3′ (PPT-adapter1)-atttgaacaccacacctcga 5′
(sequencing primer)
or
(SEQ ID NO: 2)
3′ (invertedT-adapter1)-atttgaacaccacacctcga 5′
(sequencing primer)

Step 12. Adapter 2 can include a >15-nt barcode sequence so that fluorescent hybridization can be used for determining the specific sequence that is incorporated into the final rSBL product. The barcode length for can be 1 or 2-bases for in situ sequencing readout using optical microscopy.

For additional codons, Step 1-12 is iterated. For 50 codons, this procedure generates 50 phosphothiolated target specific primers (20-35-nt+adapter sequence) and 150 partially degenerate rSBL probes (12-nt+adapter sequence), including wild-type sequence competitors. If nonsense mutations are considered in addition to missense mutations, the final number of partially degenerate rSBL probes may change.

### Discussion of Example 9

A practical result of the method exemplified in Example 8 is the creation of a generic cancer probe with high single-base specificity and sensitivity, capable of labeling cells based on common driver mutations rather than functional biomarkers that require extensive testing and validation. Our algorithm results in a set of pancreatic ductal adenocarcinoma (PDA)-specific probes capable of sequencing seven Kras mutations that account for 86% of PDAs. Our algorithm enables the detection of up to 112 non-synonymous somatic mutation variants de novo using 23 oligonucleotides as shown in Table 3 in a single-pot reaction. The algorithm can be broadly generalized for creating multiple cancer-specific probe panels or a pan-cancer probe panel for labeling, visualizing, and isolating human cancers cells. Each probe cancer-specific panel can be combined with SBL and signal amplification reagents described for various medical and research purposes.

TABLE 3
23 probes for sequencing seven Kras
mutations that account for 86% of PDAs.

| | | | | rSBL primers |
|---|---|---|---|---|
| | | | Sequencing Primer | (bold = PCR |
| | | | (* = phosphorothioate | adapter |
| | | | bond, | sequence, in |
| | | | bold = PCR adapter | situ PCR adapter |
| | | | sequence, | sequence, or |
| | | | in situ | in situ signal |
| | | | PCR adapter | amplification |
| | | | sequence, | handle, |
| | Amino | | or in situ signal | italic = |
| Mutation | Acid | Codon | amplification | Interrogated |
| Name | Change | Change | handle) | codon) |
| G12A | Glycine> | ACC> | /Phos/AGCTCCAACTACCACAAGTT | |
| | Alanine | ATC | CAGGATACACACTACCC*G*T*G | |
| | | | (SEQ ID NO: 9) | |
| Mixed | | | | GGGTCATATCGGTCACTGTT |
| | | | | GCCTACGCCNNN |
| | | | | (SEQ ID NO: 10) |
| 1 | | | | GGGTCATATCGGTCACTGTT |
| | | | | GCCTACGCCADC |
| | | | | (SEQ ID NO: 11) |
| 2 | | | | GGGTCATATCGGTCACTGTT |
| | | | | GCCTACGCCADC |
| | | | | (SEQ ID NO: 12) |
| 3 | | | | GGGTCATATCGGTCACTGTT |
| | | | | GCCTACGCCACD |
| | | | | (SEQ ID NO: 13) |
| 4 | | | | GCCTACGCCACC |
| | | | | (SEQ ID NO: 14) |
| Q61K | Glutatmine> | CAA> | /Phos/ACCTGCTGTGTCGAGAATAT | |
| | Lysine | AAA | CAGGATACACACTACCC*G*T*G | |
| | | | (SEQ ID NO: 15) | |
| Mixed | | | | GGGTCATATCGGTCACTGTT |
| | | | | GTACTCCTCNNN |
| | | | | (SEQ ID NO: 16) |
| 5 | | | | GGGTCATATCGGTCACTGTT |
| | | | | GTACTCCTCVTG |
| | | | | (SEQ ID NO: 17) |
| 6 | | | | GGGTCATATCGGTCACTGTT |
| | | | | GTACTCCTCTVG |
| | | | | (SEQ ID NO: 18) |
| 7 | | | | GGGTCATATCGGTCACTGTT |
| | | | | GTACTCCTCTTH |
| | | | | (SEQ ID NO: 19) |
| 8 | | | | GTACTCCTCTTG |
| | | | | (SEQ ID NO: 20) |
| A146P | Alanine> | GCA> | /Phos/TGATGTTTCAATAAAAGGAAT | |
| | Proline- | CCA | CAGGATACACACTACCC*G*T*G | |
| | | | (SEQ ID NO: 21) | |
| Mixed | | | | GGGTCATATCGGTCACTGTT |
| | | | | TCTTGTCTTNNN |
| | | | | (SEQ ID NO. 22) |
| 9 | | | | GGGTCATATCGGTCACTGTT |
| | | | | TCTTGTCTTVGC |
| | | | | (SEQ ID NO: 23) |
| 10 | | | | GGGTCATATCGGTCACTGTT |
| | | | | TCTTGTCTTTHC |
| | | | | (SEQ ID NO: 24) |
| 11 | | | | GGGTCATATCGGTCACTGTT |
| | | | | TCTTGTCTTTGD |
| | | | | (SEQ ID NO: 25) |
| 12 | | | | TCTTGTCTTTGC |
| | | | | (SEQ ID NO: 26) |
| G13C | Gycine> | GGC> | /Phos/ACCAGCTCCAACTACCACAA | |
| | Cysteine | TGC | CAGGATACACACTACCC*G*T*G | |
| | | | (SEQ ID NO: 27) | |
| Mixed | | | | GGGTCATATCGGTCACTGTT |
| | | | | CTTGCCTACNNN |
| | | | | (SEQ TD NO: 28) |
| 13 | | | | GGGTCATATCGGTCACTGTT |
| | | | | CTTGCCTACDGG |
| | | | | (SEQ ID NO: 29) |
| 14 | | | | GGGTCATATCGGTCACTGTT |
| | | | | CTTGCCTACCHG |
| | | | | (SEQ ID NO: 30) |
| 15 | | | | GGGTCATATCGGTCACTGTT |
| | | | | CTTGCCTACCGH |
| | | | | (SEQ ID NO: 31) |
| 16 | | | | CTrGCCTACCGG |
| | | | | (SEQ ID NO: 32) |
| G12, 13 | | | /Phos/AGCTCCAACTACCACAAGTT | |
| (adjacent) | | | CAGGATACACACTACCC*G*T*G | |
| | | | (SEQ ID NO: 33) | |
| Mixed | | | | GGGTCATATCGGTCACTGTT |
| | | | | GCCTACNNNNNN |
| | | | | (SEQ ID NO: 34) |
| 17 | | | | GGGTCATATCGGTCACTGTT |
| | | | | GCCTACHCCACC |
| | | | | (SEQ ID NO: 35) |
| 18 | | | | GGGTCATATCGGTCACTGTT |
| | | | | GCCTACGDCACC |
| | | | | (SEQ ID NO: 36) |
| 19 | | | | GGGTCATATCGGTCACTGTT |
| | | | | GCCTACGCDACC |
| | | | | (SEQ ID NO: 37) |
| 20 | | | | GGGTCATATCGGTCACTGTT |
| | | | | GCCTACGCCBCC |
| | | | | (SEQ ID NO: 38) |
| 21 | | | | GGGTCATATCGGTCACTGTT |
| | | | | GCCTACGCCBCC |
| | | | | (SEQ ID NO: 39) |
| 22 | | | | GGGTCATATCGGTCACTGTT |
| | | | | GCCTACGCCACD |
| | | | | (SEQ ID NO: 40) |
| 23 | | | | GCCTACGCCACC |
| | | | | (SEQ ID NO: 41) |

### Example 10—rSBL on RNA Attached to Magnetic Beads or Fixed Cell Suspension

Synthetic RNA transcripts of 42 bases long are obtained with 16 different ligation junctions located at bases 30 and 31. The RNA is biotinylated at the 3′ end. DNA probes are designed with the sequencing primer being complementary with a hybridization size of 30 and a 3′-FAM fluorophore, as well as 5′ phosphate. The forward primer is obtained with each base combination at the 3′ end, with the rest of the 11 bases being complementary. This is a total of six oligos being obtained. Entire workspace is cleaned to ensure RNase-free reaction. RNA template is added at 5-uM and DNA sequencing primer at twice the concentration of the RNA template, 10-uM, in 2×SSC to a total volume of 50-uL. Oligos are mixed via gentle pipetting up and down. Oligo mixture is then incubated at 95° C. for 5 minutes, 60° C. for 10 minutes, and room temperature for 10 minutes. While the incubation is occurring, 50-uL of Dynabeads streptavidin m270 (ThermoFisher, 65306) per each 50-uL volume of oligos were washed in 2×SSC 3-4 times depending on the volume of beads. After oligos are cooled to room temperature, they are added to the washed beads and shaken with gentle agitation for 15 minutes at room temperature. After 15 minutes the beads are placed on a magnetic stand until the supernatant became clear (2 minutes), the supernatant is removed. Beads with oligos are then washed three times with 10-mM Tris buffer. Beads are then split into two aliquots for positive and negative controls. A SplintR master mix consisting of 20-uM of forward primer per base for a total of 80-uM of forward primers, 1.0-uM of SplintR Ligase (NEB, M0375L) and 1× SplintR buffer to a total volume of 20-uL per reaction. Master mix is added to washed beads, mixed gently, and incubated at 37° C. for 60 mins with a 10-minute heat kill at 70° C. post ligation. Beads were then washed with 10 mM Tris buffer three times. RNase cocktail of 6.25 U of RNase H (Enzymatics, Y9220L), 2× RNase H buffer, 20-ug of RNase DNase-free (Sigma Aldrich 11119915001) and ultrapure water to 50-uL per reaction. Cocktail is added to beads and incubated at 37° C. for 1 hour followed by 10 minutes of 70° C. Supernatant is removed and diluted 1:30 in ultrapure water. 3-uL of dilution is added to 9-uL of HiDi Formamide (ThermoFisher, 4311320) and 0.5-uL of GeneScan ROX 500 (ThermoFisher, 401734) per reaction. Mixture is to be heated to 95° C. for 5 minutes, placed on ice, and centrifuged for 5 seconds. Plate is then run in Bioanalyzer AB13730, GeneMapper software was is to analyze data. Downstream NGS preparation included PCR amplification with 0.25-uL of each primer, 6.0-uL of ligation product, 25.0-uL of Phusion Pfu high fidelity mastermix (NEB M0531s) and ultrapure water to 50-uL. Amplified product is cleaned with 2% SizeSelect E-Gel (ThermoFisher G661012) and proceeded with NEB Next Ultra II Library Prep Kit for Illumina (NEB E7645s and E7335s) as per commercial protocol. All Primers formulated as desalted.

Sequencing Primer:
(SEQ ID NO: 42)
pAGATGGGACCTACAATGTACCAGAAGCGTCTATGA
CACTC-FAM 3′
Forward Primers:
(SEQ ID NO: 43)
CGATCGTCTACTTCTATTGA
(SEQ ID NO: 44)
CGATCGATCGATCGTCTACTTCTATTGC
(SEQ ID NO: 45)
GATCGATCGATCGATCGATCGTCTACTTCTATTGG
(SEQ ID NO: 46)
ATCGATCGATCGATCGATCGATCGATCGTCTACTTC
TATTGT
RNA template:
(SEQ ID NO: 47)
rGrArC rGrCrU rUrCrU rGrGrU rArCrA
rUrUrG rUrArG rGrUrC rCrCrA rUrCrU
rUrCrA rArUrA rGrArA rGrU

### Example 11—Multiplexed rSBL for Genotyping Using qPCR or NGS

96 sequencing primers are added at 10-uL each (100 uM stock concentration) for a total of 960-uL of primers. A phosphorylation master mix was then made with 10-ul 10× T4 DNA ligase buffer (NEB Catalog B0202S), 50 U of PNK enzyme (NEB Catalog M0201S), 25-uL of sequencing primer mix (stock 100-uM, final concentration 25-uM), and ultrapure water to 100-uL per reaction. The mix is then incubated at 37° C. for 1 hour and heat inactivated at 65° C. for 20 minutes. Cells are then lysed on plate in 50-uL of Single Shot Lysis Buffer (BioRad Catalog 1725080) at ˜-100,000 cells per 50-uL following the manufacturer's protocol. Alternatively, the lysate is incubated with poly dT oligonucleotides and streptavidin magnetic beads as followed by mRNA isolation on beads. 5-uL of the lysate is then added to 5-uL of the sequencing primer mix (25-uM, 5-uM final concentration), 10-uL of the phosphorylated degenerate forward primer mix (100-uM, 40-uM final concentration), and 2.5-uM of 10× SplintR buffer (NEB Catalog M0375S). For bead-based protocols, the sample excess probes and reagents are decanted, and the sample is washed twice in the wash buffer. Mixture is then heated to 95° C. for 5 minutes, 60° C. for 10 minutes, room temperature for 10 minutes and held at 4° C. 2.5-uL of SplintR ligase (NEB Catalog M0375S) was then added to each reaction, or 2.5-uL of water for negative controls. The ligation mixture is then incubated at 37° C. for 1 hour and heat inactivated at 70° C. for 10 minutes. To quantify, 1-uL of 10,000× diluted product is added to 5-uL of PowerUp Sybr Green Master Mix (Thermofisher Catalog A25742), 0.1-uL of 100-uM primers and 3.8-uL of ultrapure water. The mix is run on a Quant Studio qPCR machine and analyzed. Downstream NGS preparation included PCR amplification with 0.25-uL of each primer, 6.0-uL of ligation product, 25.0-uL of Phusion Pfu high fidelity mastermix (NEB M053 is) and ultrapure water to 50-uL. Amplified product is cleaned with 2% SizeSelect E-Gel (ThermoFisher G661012) and proceeded with NEB Next Ultra II Library Prep Kit for Illumina (NEB E7645s and E7335s) as per commercial protocol. All Primers formulated as desalted.

qPCR primers (IDT):

| | | SEQ |
|---|---|---|
| | | ID |
| Name | Sequence | NO: |
| AP1 | GGGTCATATCGGTCACTGTT | 48 |
| AP2 | CACGGGTACTGTGTATCCTG | 49 |
| Forward | GGGTCATATCGGTCACTGT | 50 |
| Sequencing | TNNNNNNNNNNNN | |
| primer | | |
| (IDT): | | |

TABLE 4
96 sequencing primers flanking
human SNPs (IDT)

| | | | SEQ |
|---|---|---|---|
| | | | ID |
| # | Name | Sequence | NO: |
| 01 | rs13212638 | AGCAGCTGTG | 51 |
| | | TCCATGCAGC | |
| | | CAGGATACAC | |
| | | ACTACCCGTG | |
| 02 | rs656566 | ATGTTTGTGA | 52 |
| | | TGTGCCCGGT | |
| | | CAGGATACAC | |
| | | ACTACCCGTG | |
| 03 | rs2710673 | AAACATAATC | 53 |
| | | CTCAGGTATC | |
| | | CAGGATACAC | |
| | | ACTACCCGTG | |
| 04 | rs1042078 | AATGTGCTTT | 54 |
| | | CAATTGATGG | |
| | | CAGGATACAC | |
| | | ACTACCCGTG | |
| 05 | rs3796133 | TACTGTTCTT | 55 |
| | | GAAATACCTA | |
| | | CAGGATACAC | |
| | | ACTACCCGTG | |
| 06 | rs382212S | ACACACAATT | 56 |
| | | CATCAAGCAC | |
| | | CAGGATACAC | |
| | | ACTACCCGTG | |
| 07 | rs900654 | TTCCTGAGGT | 57 |
| | | TCTGCATCCA | |
| | | CAGGATACAC | |
| | | ACTACCCGTG | |
| 08 | rs11168371 | ATTCTTTCCA | 58 |
| | | GCATTGTGCT | |
| | | CAGGATACAC | |
| | | ACTACCCGTG | |
| 09 | rs5749426 | TGACCCCAAT | 59 |
| | | AAAGTTTCTG | |
| | | CAGGATACAC | |
| | | ACTACCCGTG | |
| 10 | rs1046383 | AGCACTGAGG | 60 |
| | | TGGGGGAGCA | |
| | | CAGGATACAC | |
| | | ACTACCCGTG | |
| 11 | rs9444701 | ACAGTGAAGA | 61 |
| | | CAAGAATGGT | |
| | | CAGGATACAC | |
| | | ACTACCCGTG | |
| 12 | rs6847 | TTTGAGCTTT | 62 |
| | | GAACACTGAA | |
| | | CAGGATACAC | |
| | | ACTACCCGTG | |
| 13 | rs13509 | AAAGAAAAAC | 63 |
| | | CCAATGTAAA | |
| | | CAGGATACAC | |
| | | ACTACCCGTG | |
| 14 | rs4856 | TTATTTCCAA | 64 |
| | | ATACTGAGAC | |
| | | AGGATACACA | |
| | | CTACCCGTG | |
| 15 | rs284856 | AAAACCACAG | 65 |
| | | ATAACCAAGG | |
| | | CAGGATACAC | |
| | | ACTACCCGTG | |
| 16 | rs6664 | TTTAGAAGAA | 66 |
| | | TGCCTCCTCG | |
| | | CAGGATACAC | |
| | | ACTACCCGTG | |
| 17 | rs1054471 | TTGTACTAAC | 67 |
| | | TTATGATAGA | |
| | | CAGGATACAC | |
| | | ACTACCCGTG | |
| 18 | rs10864033 | AAGGAGCOGG | 68 |
| | | TGTCGGAGAA | |
| | | CAGGATACAC | |
| | | ACTACCCGTG | |
| 19 | rs2794768 | AGCTGTGGGC | 69 |
| | | ACATTATGTA | |
| | | CAGGATACAC | |
| | | ACTACCCGTG | |
| 20 | rs11714353 | AGTCCTTTGA | 70 |
| | | GGGGACAGAT | |
| | | CAGGATACAC | |
| | | ACTACCCGTG | |
| 21 | rs13521 | TGCCTGCAGC | 71 |
| | | CTTCATAAGC | |
| | | CAGGATACAC | |
| | | ACTACCCGTG | |
| 22 | rs3288S666 | AGCCATGGAG | 72 |
| | | TGGGCACTAC | |
| | | CAGGATACAC | |
| | | ACTACCCGTG | |
| 23 | rs1059672 | TCGGTTTTGC | 73 |
| | | CGGTTTCTTT | |
| | | CAGGATACAC | |
| | | ACTACCCGTG | |
| 24 | rs17689863 | ACGCTTCAAT | 74 |
| | | TTCCTTCCAT | |
| | | CAGGATACAC | |
| | | ACTACCCGTG | |
| 25 | rs12030 | AAGCTGCCAA | 75 |
| | | AGAACACATC | |
| | | CAGGATACAC | |
| | | ACTACCCGTG | |
| 26 | rs1130569 | ATGTCATGAA | 76 |
| | | AGATTTTGAG | |
| | | CAGGATACAC | |
| | | ACTACCCGTG | |
| 27 | rs308998 | TCTGTCCATG | 77 |
| | | ATGTCAAAAG | |
| | | CAGGATACAC | |
| | | ACTACCCGTG | |
| 28 | rs368322 | TGTACACTGA | 78 |
| | | GGTAGGAAAT | |
| | | CAGGATACAC | |
| | | ACTACCCGTG | |
| 29 | rs1045056 | AGCGCTTGGT | 79 |
| | | CTGTGTCCTT | |
| | | CAGGATACAC | |
| | | ACTACCCGTG | |
| 30 | rs8756 | TAGCTGCGAC | 80 |
| | | CAACAACAGC | |
| | | CAGGATACAC | |
| | | ACTACCCGTG | |
| 31 | rs763121 | AATCATAAAA | 81 |
| | | TAACAGTAAA | |
| | | CAGGATACAC | |
| | | ACTACCCGTG | |
| 32 | rs12695 | TCATCCCCAA | 82 |
| | | GCCCCTCAAG | |
| | | CAGGATACAC | |
| | | ACTACCCGTG | |
| 33 | rs1051854 | ACCCTCTTAA | 83 |
| | | ATGTCAAAGA | |
| | | CAGGATACAC | |
| | | ACTACCCGTG | |
| 34 | rs2679745 | TTCTCATCCT | 84 |
| | | TTCTCGCTCT | |
| | | CAGGATACAC | |
| | | ACTACCCGTG | |
| 35 | rs86796 | TGCCCTGCAC | 85 |
| | | ATTTTCTTTT | |
| | | CAGGATACAC | |
| | | ACTACCCGTG | |
| 36 | rs1131636 | AAGTAAAGAA | 86 |
| | | ACGCTTTGTA | |
| | | CAGGATACAC | |
| | | ACTACCCGTG | |
| 37 | rs3476 | AAAGTGTCAT | 87 |
| | | CAATTTGTAA | |
| | | CAGGATACAC | |
| | | ACTACCCGTG | |
| 38 | rs700006 | AGCTTGGGAG | 88 |
| | | CCACATGGCT | |
| | | CAGGATACAC | |
| | | ACTACCCGTG | |
| 39 | rs1803183 | AGCTTGATCA | 89 |
| | | CCACCGCCTT | |
| | | CAGGATACAC | |
| | | ACTACCCGTG | |
| 40 | rs3209874 | TGGCCTTCAG | 90 |
| | | AAGCATCTCC | |
| | | CAGGATACAC | |
| | | ACTACCCGTG | |
| 41 | rs4229 | AAATCACCTG | 91 |
| | | CAGCTAAGCA | |
| | | CAGGATACAC | |
| | | ACTACCCGTG | |
| 42 | rs4933734 | AAGTGGGCTT | 92 |
| | | TTTGTGAACT | |
| | | CAGGATACAC | |
| | | ACTACCCGTG | |
| 43 | rs1010 | TGGCAGCAGG | 93 |
| | | GCCTCGGGAA | |
| | | CAGGATACAC | |
| | | ACTACCCGTG | |
| 44 | rs7314 | AGAATTAAAA | 94 |
| | | AGCTTTTTTT | |
| | | CAGGATACAC | |
| | | ACTACCCGTG | |
| 45 | rs6793 | TTAACTTGCC | 95 |
| | | CAAAGTTCAC | |
| | | CAGGATACAC | |
| | | ACTACCCGTG | |
| 46 | rs7436 | AAAAAGTTGT | 96 |
| | | AATAGAGAAT | |
| | | CAGGATACAC | |
| | | ACTACCCGTG | |
| 47 | rs4804 | TCCTTGATGT | 97 |
| | | CAAAATGGGG | |
| | | CAGGATACAC | |
| | | ACTACCCGTG | |
| 48 | rs583121 | AGACCCACCC | 98 |
| | | ATAAGGCTGC | |
| | | CAGGATACAC | |
| | | ACTACCCGTG | |
| 49 | rs6994686 | ACGCCTTCTC | 99 |
| | | CAACAAAAGA | |
| | | CAGGATACAC | |
| | | ACTACCCGTG | |
| 50 | rs1435! | ACCAACAGAC | 100 |
| | | CCCAATTTCC | |
| | | CAGGATACAC | |
| | | ACTACCCGTG | |
| 51 | rs22356t1 | ATCGGGACCG | 101 |
| | | AGACCTGCTT | |
| | | CAGGATACAC | |
| | | ACTACCCGTG | |
| 52 | rs3192243 | TTTTTAAGAG | 102 |
| | | CAAATTCTGT | |
| | | CAGGATACAC | |
| | | ACTACCCGTG | |
| 53 | rs3549 | TTAGAGATAG | 103 |
| | | AGAAACAGAC | |
| | | CAGGATACAC | |
| | | ACTACCCGTG | |
| 54 | rs3048 | TTGGTAGTCA | 104 |
| | | TGTCTTTGTG | |
| | | CAGGATACAC | |
| | | ACTACCCGTG | |
| 55 | rs4235 | TTCTTCTCTG | 105 |
| | | CTGCATTTGG | |
| | | CAGGATACAC | |
| | | ACTACCCGTG | |
| 56 | rs1757095 | TCGCTGTGTG | 106 |
| | | AATGGGCAGT | |
| | | CAGGATACAC | |
| | | ACTACCCGTG | |
| 57 | rs4625 | ACAGAGATGC | 107 |
| | | AGATGGACGG | |
| | | CAGGATACAC | |
| | | ACTACCCGTG | |
| 58 | Rs1051055 | TGATGTCCAC | 108 |
| | | ACTGCTCGGC | |
| | | CAGGATACAC | |
| | | ACTACCCGTG | |
| 59 | rs14868 | AAAGTGGTGA | 109 |
| | | GGAGAAAACA | |
| | | CAGGATACAC | |
| | | ACTACCCGTG | |
| 60 | rs9031 | TGATGGAAGC | 110 |
| | | AGCGGAGGCC | |
| | | CAGGATACAC | |
| | | ACTACCCGTG | |
| 61 | rs7473 | AGAGCAAGGC | 111 |
| | | TGTAGAGATT | |
| | | CAGGATACAC | |
| | | ACTACCCGTG | |
| 62 | rs7775 | AACTAAATCC | 112 |
| | | CGAAATACAA | |
| | | CAGGATACAC | |
| | | ACTACCCGTG | |
| 63 | rs8905 | AGGATAGCCT | 113 |
| | | TTCAGACCAA | |
| | | CAGGATACAC | |
| | | ACTACCCGTG | |
| 64 | rs1947 | ACGCCCCACC | 114 |
| | | TGCCACCCTC | |
| | | CAGGATACAC | |
| | | ACTACCCGTG | |
| 65 | rs10253347 | AGCACTACCA | 115 |
| | | TGCAGGGTAC | |
| | | CAGGATACAC | |
| | | ACTACCCGTG | |
| 66 | rs11486 | TAAAAGTGTA | 116 |
| | | ATAATGGAAA | |
| | | CAGGATACAC | |
| | | ACTACCCGTG | |
| 67 | rs8575 | TGCTCGCAGT | 117 |
| | | GGGCTGATTC | |
| | | CAGGATACAC | |
| | | ACTACCCGTG | |
| 68 | rs3011623 | AGGGGAGTGA | 118 |
| | | TTTAAGCAAT | |
| | | CAGGATACAC | |
| | | ACTACCCGTG | |
| 69 | rs254682 | ATTCTAGAGT | 119 |
| | | TTGGAATGCA | |
| | | CAGGATACAC | |
| | | ACTACCCGTG | |
| 70 | rs7791181 | ACTGTGCTAG | 120 |
| | | GCTATCCAAG | |
| | | CAGGATACAC | |
| | | ACTACCCGTG | |
| 71 | rs7295 | AAAGTTTATA | 121 |
| | | AACAAAGCTC | |
| | | CAGGATACAC | |
| | | ACTACCCGTG | |
| 72 | rs6099216 | ACACTCACCT | 122 |
| | | TAGGGTTCAG | |
| | | CAGGATACAC | |
| | | ACTACCCGTG | |
| 73 | rs17074615 | TTGACATCAA | 123 |
| | | GAAAAGACTA | |
| | | CAGGATACAC | |
| | | ACTACCCGTG | |
| 74 | Rs7115 | ATCCACTGGT | 124 |
| | | ACTGCAGGTT | |
| | | CAGGATACAC | |
| | | ACTACCCGTG | |
| 75 | Rs7177445 | TGACTGGCCC | 125 |
| | | ACACGTGCAT | |
| | | CAGGATACAC | |
| | | ACTACCCGTG | |
| 76 | rs4239 | AACCCTGGGT | 126 |
| | | CAAAAAGAGA | |
| | | CAGGATACAC | |
| | | ACTACCCGTG | |
| 77 | rs2287926 | ACTAGGTGTG | 127 |
| | | GGTAATTTGG | |
| | | CAGGATACAC | |
| | | ACTACCCGTG | |
| 78 | rs3816593 | AGGATCAGAA | 128 |
| | | ACAGCTTTGG | |
| | | CAGGATACAC | |
| | | ACTACCCGTG | |
| 79 | rs1045215 | AGTGCTTTGT | 129 |
| | | AGTCTCTCCT | |
| | | CAGGATACAC | |
| | | ACTACCCGTG | |
| 80 | rs1131312 | TCCCTGACAC | 130 |
| | | TTTAACCTCA | |
| | | CAGGATACAC | |
| | | ACTACCCGTG | |
| 81 | rs1043SSI | TAGACACATT | 131 |
| | | CTTTATTATT | |
| | | CAGGATACAC | |
| | | ACTACCCGTG | |
| 82 | rs8386 | TTCTCAGTGT | 132 |
| | | CCACAGCGCA | |
| | | CAGGATACAC | |
| | | ACTACCCGTG | |
| 83 | rs162549 | TTTAATATGC | 133 |
| | | TTATAACCTA | |
| | | CAGGATACAC | |
| | | ACTACCCGTG | |
| 84 | rs17052849 | TTAAAGACCC | 134 |
| | | TGAGTTATGT | |
| | | CAGGATACAC | |
| | | ACTACCCGTG | |
| 85 | rs13689 | AACTTCTTAC | 135 |
| | | CCTAAAAGAG | |
| | | CAGGATACAC | |
| | | ACTACCCGTG | |
| 86 | rs4988291 | TTTTAAAATA | 136 |
| | | CTAGCCTGTA | |
| | | CAGGATACAC | |
| | | ACTACCCGTG | |
| 87 | rs1045115 | AAACTCAGGA | 137 |
| | | AAACCTCTCA | |
| | | CAGGATACAC | |
| | | ACTACCCGTG | |
| 88 | rs6541 | TTCTCTCCCG | 138 |
| | | TATCACCTAA | |
| | | CAGGATACAC | |
| | | ACTACCCGTG | |
| 89 | rs4800836 | AAAAAAAAAA | 139 |
| | | TGGAAAAAAA | |
| | | CAGGATACAC | |
| | | ACTACCCGTG | |
| 90 | rs7297351 | AAAAACAAGC | 140 |
| | | TGGATAACCA | |
| | | CAGGATACAC | |
| | | ACTACCCGTG | |
| 91 | rs1866374 | TGGCTTGTCT | 141 |
| | | TTTTTCACCA | |
| | | CAGGATACAC | |
| | | ACTACCCGTG | |
| 92 | rs2240172 | TCTCAGTGTT | 142 |
| | | TCAACCTCAC | |
| | | CAGGATACAC | |
| | | ACTACCCGTG | |
| 93 | rs12745610 | AAAGTCTCGT | 143 |
| | | GCACATGTGA | |
| | | CAGGATACAC | |
| | | ACTACCCGTG | |
| 94 | rs6864342 | TGATGGTGCT | 144 |
| | | CAGATTGCAA | |
| | | CAGGATACAC | |
| | | ACTACCCGTG | |
| 95 | rs1561743 | TGCAACAAAA | 145 |
| | | GATTAGAACA | |
| | | CAGGATACAC | |
| | | ACTACCCGTG | |
| 96 | rs2657 | TTAGTATATA | 146 |
| | | GAAATAATAC | |
| | | CAGGATACAC | |
| | | ACTACCCGTG | |

### Discussion of Examples 1-11

In conclusion, the above examples demonstrate a flexible and scalable platform for detecting or sequencing RNA single-nucleotide variants with sensitivity and specificity surpassing existing single-cell methods. In addition, the incorporation of signal amplification of molecular, cellular, or pathway identifiers across many different tissue types and applications, including isolating rare single cells based on somatic mutations. Because of its simplicity, the platform can be adapted for ‘staining’ clinical tissue specimens using their genetic characteristics, including point mutations, translocations, and tumor type gene expression markers. Fundamentally, the platform is a nucleotide-specific targeted in situ amplification method compatible with multiple downstream applications, including single cell genomics, in situ hybridization, and in situ sequencing methods. More specifically, the technology can be used to mark the position of individual cells prior to dissociation-dependent single cell analysis or to improve the detection sensitivity of in situ sequencing methods. By incorporating gel encapsulation and probe immobilization techniques, its spatial resolution can be improved even further. The platform, named Heuristic In Situ Targeted Oligopaint sequencing (HISTO-seq) enables the development of applications for disease-specific genetic ‘dyes’ for uses in basic research or clinical applications.

### Example 12: Target Amplification Using Programmable rSBL Probes

In this example, 100 ng 50-mer 5′ biotinylated RNA templates are bound to Dynabeads (ThermoFisher) in a provided binding buffer at 25° C. for 10 minutes, followed by a wash cycle in 2×SSC. The 5′ phosphorylated 20-mer DNA sequence primer with a 3′ FITC modification (IDT) is added in 3-fold molar excess for DNA-RNA hybridization in 2×SSC with RNaseOUT (ThermoFisher) for 10 min at 60° C. After two rounds of washing cycles using 2×SSC, 2 U PBCV DNA ligase (SplintR, NEB) along with 10-fold molar excess of programmable rSBL probes are added to the DNA-RNA complex bound to Dynabeads in the SplintR reaction buffer containing RNaseOUT. The reaction is incubated at 37° C. for 60 minutes and washed twice using 2×SSC. The immobilized RNA is degraded using 1 U RNase H (NEB) and RNase A (NEB) in the ligation buffer, releasing the FITC-labeled sequencing primer and the full rSBL product. After spinning in microcentrifuge, the supernatant is collected and diluted using ddH2O 100-fold for fragment size analysis on ABI 3730 capillary sequencer. The area under the FITC signal is quantified as well as the position of individual peaks reflecting the size of ligated or un-ligated products. The size of base-specific rSBL products differ in fragment size by 5-10 bases, enabling the discrimination of correct to incorrect incorporation of interrogating oligonucleotides. The ligation efficiency of correct rSBL is expressed as (Area under the correct rSBL product)/(Area under the un-ligated FITC primer+Area under the incorrectly ligated rSBL product). When the 5′ base of the sequencing primer is A or T, the ligation efficiency is >90% after 60 minutes of rSBL at 37° C.

After rSBL the ligation product in the supernatant is used for PCR, qPCR, digital droplet PCR, or in situ PCR/RCA/MDA on a flow cell. In this example, the ligation product in the supernatant after RNase H and RNase A digestion is diluted in ddH2O 1 to 1,000-fold, depending on the starting amount of immobilized RNA template. For 100 ng RNA template, the rSBL product was diluted 1,000-times in ddH2O. Two microliters of the diluted product are added to KAPA Real-Time Sybr-Green qPCR 2× Master Mix, along with 10 μM forward and reverse PCR primers against Adapter 1 and Adapter 2 sequences in the rSBL product. The cycling parameters are as follows: 95° C. for 30 sec, 60° C. for 10 sec, and 72° C. for 10 sec for 40 cycles. The real-time qPCR benchtop instrument (Eppendorf) is used to quantify the rate of PCR amplification to estimate the amount of rSBL products using un-ligated and wild-type reference samples for ΔΔCt calculations. The final product size (85-nt) was validated using 2% agarose gel electrophoresis.

### Example 13: Cell Labeling Using Programmable rSBL Probes for FACS Analysis or Imaging

In this example, single cells of interest from the blood (Ficoll centrifugation) or enzymatic tissue dissociation (trypsin) are fixed in 4% PFA in PBS-T at 4° C. for 15 min. Cells are pelleted using 100-g centrifugation over 15 min at 4° C., and washed in cold DEPC-PBS twice. The 5′ phosphorylated 50-mer DNA sequencing primer (25-nt target specific sequence+20-nt adapter sequence; 1 uM) are added for in situ RNA hybridization in 2×SSC with RNaseOUT (ThermoFisher) for 2 hours to overnight at 42 to 60° C., depending on cell type. After two rounds of washing cycles using 2×SSC, 2 U PBCV DNA ligase (SplintR, NEB) along with 20-uM programmable rSBL probes are added to the fixed cells in the SplintR reaction buffer containing RNaseOUT. The reaction is incubated at 37° C. for 60 minutes and washed twice using 2×SSC. Un-ligated rSBL probes are degraded by 1 U Exonuclease I/I at 37° C. for 1 hour, while ligated rSBL products survive enzymatic digestion due to phosphothioate modifications in the sequencing primer. Individual cells are stabilized further using degassed 4% polyacrylamide (no bis-acrylamide) solution with APS and TEMED for 1 hour. Single-cell-hydrogel particles are filtered through a 200-um nylon mesh to eliminate large particle aggregates. The collected single-cell hydrogel mixtures are added to KAPA PCR Master Mix with forward and reverse PCR primers against adapter sequence 1 and 2. Cycling parameters can start follows: 95° C. for 30 sec, 60° C. for 30 sec, and 72° C. for 30 sec for 10-30 cycles for in situ PCR. The resulting double-stranded PCR products are converted into single-stranded DNA using lambda 5′ exonuclease at 37° C. for 1 hour in the provided buffer, followed by re-fixation with 4% PFA in PBS prior to fluorescent FISH probe hybridization in 2×SSC. The labeled cells are then ready for FACS analysis.

In this example adherent cells or fresh frozen tissue sections on a glass slide are fixed in 4% PFA prior to rSBL. Silicone gaskets (Grace-bio) are cut to size (˜10-mm chamber diameter) and placed to enclose the specimen, forming an open flow-cell accessible to direct manipulation. The 5′ phosphorylated 50-mer DNA sequencing primer (25-nt target specific sequence+20-nt adapter sequence; 1 uM) are used for in situ RNA hybridization in 2×SSC with RNaseOUT for overnight at 42° C. After two rounds of washing cycles using 2×DEPC-SSC, 2 U PBCV DNA ligase along with 20-uM programmable rSBL probes are added to fixed cells or tissue sections in the SplintR reaction buffer containing RNaseOUT. The reaction is incubated at 37° C. for 60 minutes and washed twice using 2×SSC. Un-ligated rSBL probes are degraded by 1 U Exonuclease 11111 at 37° C. for 1 hour, while ligated rSBL products survive enzymatic digestion due to phosphothioate modifications in the sequencing primer. The fixed cells or tissues are incubated with KAPA PCR Master Mix with 5′ phosphorylated forward and non-phosphorylated reverse PCR primers against adapter sequence 1 and 2. Cycling parameters can start follows: 95° C. for 30 sec, 60° C. for 30 sec, and 72° C. for 30 sec for 10-30 cycles for in situ PCR. The resulting double-stranded PCR products are converted into single-stranded DNA using lambda 5′ exonuclease at 37° C. for 1 hour in the provided buffer, followed by re-fixation with 4% PFA in PBS prior to fluorescent FISH probe hybridization in 2×SSC. The labeled cells are then ready for FACS analysis.

In this example, rSBL products are amplified using RCA rather than in situ PCR. Adherent cells or fresh frozen tissue sections on a glass slide are fixed in 4% PFA prior to rSBL. Silicone gaskets are cut to size and placed to enclose the specimen. The 5′ phosphorylated 50-mer DNA sequencing primer are used for in situ RNA hybridization in 2×SSC with RNaseOUT for overnight at 42° C. After two rounds of washing cycles using 2×DEPC-SSC, 2 U PBCV DNA ligase along with 20-uM programmable rSBL probes are added to fixed cells or tissue sections in the SplintR reaction buffer containing RNaseOUT. The reaction is incubated at 37° C. for 60 minutes and washed twice using 2×SSC. The sample is then washed with DEPC H2O to remove any trace of residual chloride. Two units of CircLigase II (Epicenter) in the CircLigase buffer are added to the rSBL product-containing fixed cells or tissues and incubated for 2 hours at 60° C. in a humidifier oven. The RCA primer is then hybridized to the circularized rSBL products in 2×SSC and 10% formamide solution at 60° C. for 10 min, followed by two 1 min wash in 2×SSC. Two units of Phi29 DNA polymerase along with amino-allele dUTP spike-in are added to the specimen at 30° C. for up to overnight. After RCA, the specimen is then incubated with BS(PEG)9 in PBS pH8.0 for 10 min at 25° C. to cross-link RCA products in situ. 100 uM fluorescently labeled detection FISH probes are hybridized against RCA products in 60° C. for 5 min followed by three washes in 2×SSC and imaging on an epifluorescence or confocal microscope. Note that wild type rSBL probes are included, but they end with inverted dT so that they cannot be circularized by CircLigase. Table 3 shows examples of codon specific probes used for in situ rSBL for RCA-based optical imaging.

Many different single-molecule FISH methods exist for increasing the fluorescent signal from single ssDNAs in situ. Some enable ssDNA rSBL products to be visualized, while others permit rSBL amplicons to be sequenced. Depending on applications, the precise nature of rSBL signal amplification differs; therefore, the experimental details for alternative signal amplification methods will not be summarized here for the sake of clarity.

TABLE 5
Mutated codon-specific rSBLfor in situ RCA

| | | | | | SEQ |
|---|---|---|---|---|---|
| Gene | Mutation | Probe | Sequences | | iD |
| name | name | name | (5′-3′) | Tm | NO: |
| KRAS | G12D | KrasG12_ | /5Phos/ | 62.1 | 147 |
| | | SP_ | agc | | |
| | | 35 | tccaa | | |
| | | | ccAcc | | |
| | | | acaag | | |
| | | | tttat | | |
| | | | actca | | |
| | | | gtcat | | |
| | | | ttTCT | | |
| | | | CGGGA | | |
| | | | A | | |
| KRAS | G12V | KrasG12_ | /5Inv | 32.5 | 148 |
| | | rSBL_ | ddT/C | | |
| | | 9_ | GCTGA | | |
| | | WT | AGAta | | |
| | | | cgccA | | |
| | | | CC | | |
| | | KrasG12D_ | /5Phos/ | 32.5 | 149 |
| | | rSBL_ | CGC | | |
| | | 9_ | TGAAG | | |
| | | ADC | Atacg | | |
| | | | ccADC | | |
| | | KrasG12V_ | /5Phs/ | 32.5 | 150 |
| | | rSBL_ | CGC | | |
| | | 9_ | TGAAG | | |
| | | ACD | Atacg | | |
| | | | ccACD | | |
| | | KrasG12_ | /5Phos/ | 35.9 | 151 |
| | | rSBL_ | CGCT | | |
| | | gap_ | GAAG | | |
| | | 9 | Agcct | | |
| | | | acgcc | | |
| TP53 | R273H | TP53R273_ | /5Phos/ | 63.2 | 152 |
| | | SP_ | CAC | | |
| | | 28 | CTCAA | | |
| | | | AGCTG | | |
| | | | TTCCG | | |
| | | | TCCCA | | |
| | | | GTAGA | | |
| | | | TCTCG | | |
| | | | GGAA | | |
| TP53 | R273C | TP53R273_ | /5Inv | 29.9 | 153 |
| | | rSBL_ | ddT/C | | |
| | | 10_ | GCTGA | | |
| | | WT | AGACA | | |
| | | | CAAAC | | |
| | | | ACG | | |
| | | TP53R273H_ | /5Pbo | 28.6 | 154 |
| | | rSBL_ | s/CGC | | |
| | | 10_ | TGAAG | | |
| | | ADG | ACACA | | |
| | | | AACAD | | |
| | | | G | | |
| | | TP53R273C_ | /5Pho | 28.8 | 155 |
| | | rSBL_ | s/CGC | | |
| | | 10_ | TGAAG | | |
| | | ACH | ACACA | | |
| | | | AACAC | | |
| | | | H | | |
| | | TP53R273_ | /5Pho | 31.2 | 156 |
| | | rSBL_gap _ | s/CGC | | |
| | | 10 | TGAAG | | |
| | | | AAGGC | | |
| | | | ACAAA | | |
| | | | C | | |
| TP53 | T220C | TP53T220_ | /5Pho | 61.6 | 157 |
| | | SP_26 | s/GGG | | |
| | | | (ACCA | | |
| | | | CCACA | | |
| | | | CTATG | | |
| | | | TCGAA | | |
| | | | AAGTC | | |
| | | | TCGGG | | |
| | | | AA | | |
| | | TP53T220_ | /5Inv | 34 9 | 158 |
| | | rSBL_ | ddT/C | | |
| | | 10_ | GCTGA | | |
| | | WT | AGAGC | | |
| | | | GGCTC | | |
| | | | ATA | | |
| | | TP53T220_ | /5Phos/ | 41.2 | 159 |
| | | rSBL_ | CGC | | |
| | | 10_ | TGAAG | | |
| | | AVA | AGCGG | | |
| | | | CTCAV | | |
| | | | A | | |
| | | TP53T220_ | /5Phos/ | 38.9 | 160 |
| | | rSBL_ | CGC | | |
| | | 10_ | TGAAG | | |
| | | ATB | AGCGG | | |
| | | | CTCAT | | |
| | | | B | | |
| | | TP53T220_ | /5Phos/ | 42.9 | 161 |
| | | rSBL_ | CGC | | |
| | | gap_ | TGAAG | | |
| | | 10 | ACAGG | | |
| | | | CGGCT | | |
| | | | C | | |

### Example 14: RNA Templated DNA Ligation (ProRSBL)

A 3′ biotinylated RNA template and a 5′ phosphorylated, 3′ FAM conjugated DNA oligo (sequencing primer) complementary to the hybridization site of the RNA template were mixed (5 μM and 10 μM, respectively) in 2×SSC to a total volume of 50 μL. The mixture was incubated at 95° C. for 5 min, 60° C. for 10 min, and room temperature for 10 min. In the meantime, 50 μL of Dynabeads streptavidin m270 (ThermoFisher, 65306) per each 50 μL volume of oligos were washed in 2×SSC 3-4 times depending on the volume of beads. The hybridized RNA:DNA duplex was added to the washed beads and gently agitated for 15 min at room temperature before being transferred to a magnetic stand for 2 min and the supernatant removed. The beads were then washed three times with 10 mM Tris buffer. Beads were then split into two aliquots, 75 μL and 25 μL for positive and negative controls, respectively. A SplintR master mix consisting of 20 μM of forward primer per base for a total of 80 μM of forward primers, 1.0 μM of SplintR Ligase (NEB, M0375L) and 1× SplintR buffer to a total volume of 20 μL per reaction. The master mix was added to washed beads, mixed gently, and incubated at 37° C. for 60 min with a 10 min heat kill at 70° C. post ligation. For time courses, reactions were removed from 37° C. at each time point and transferred to a separate 70° C. incubator for a 10 min heat kill. Beads were then washed with 10 mM Tris buffer three times. A RNase cocktail of 6.25 U of RNase H (Enzymatics, Y9220L), 2× RNase H buffer, 20 μg of RNase DNase-free (Sigma Aldrich 11119915001) and Ultrapure 1120 to 50 μL, per reaction. The cocktail was added to beads and incubated at 37° C. for 1 hour followed by 10 min of 70° C. The supernatant was removed and diluted 1:3 in Ultrapure H₂O. 3 μL of dilution was added to 9 μL of HiDi Formamide (ThermoFisher, 4311320) and 0.5 μL. of GeneScan ROX 500 (ThermoFisher, 401734) per reaction. The mixture was heated to 95° C. for 5 min, placed on ice, and centrifuged for 5 sec. Ligation products were then run in Bioanalyzer AB13730, and analyzed using GeneMapper. For base specificity, an RNA template with 16 different ligation junctions located at bases 30 and 31 was used. All Primers ordered from IDT and formulated as desalted.

### ProRSBL Nucleotide Bias Determination

Sequencing printers

| | | | SEQ |
|---|---|---|---|
| | Size | | ID |
| Donor | (nt) | Sequence | NO: |
| A | 40 | /5′Phos/AGATGGGACCTA | 42 |
| | | CAATGTACCAGAAGCGTCTA | |
| | | TGACACTC/3′6-FAM/ | |
| C | 42 | /5′Phos/CGATGGGACCTA | 162 |
| | | CAATGTACCAGAAGCGTCTC | |
| | | TATGACACTC/3A-FAM/ | |
| G | 44 | /5′Phos/GGATGGGACCTA | 163 |
| | | CAATGTACCAGAAGCGTCTC | |
| | | TCTATGACACTC/3′6-FAM/ | |
| T | 46 | /5′Phos/TGATGGGACCTA | 164 |
| | | CAATGTACCAGAAGCGTCTC | |
| | | TCTCTATGACACTC/3′6-FAM/ | |

Competitive probes

| | | | SEQ |
|---|---|---|---|
| | Size | | ID |
| Acceptor | (nt) | Sequence | NO: |
| A | 20 | 5′CGATCGTCTAC | 43 |
| | | TTCTATTGA3′ | |
| C | 28 | 5′CGATCGATCGA | 44 |
| | | TCGTCTACTTCTAT | |
| | | TGC3′ | |
| G | 36 | 5′CGATCGATCGAT | 165 |
| | | CGATCGATCGTCTA | |
| | | CTTCTATTGG3′ | |
| T | 44 | 5′CGATCGATCGAT | 166 |
| | | CGATCGATCGATCG | |
| | | ATCGTCTACTTC | |
| | | TATTGT3′ | |

RNA templates

| | | Sequence | |
|---|---|---|---|
| | Product | (ligation | |
| | size | junction | SEQ ID |
| Junction | (nt) | underlined) | NO: |
| UU | 60 | 5′GACGCUUC | 167 |
| | | UGGUACAUUG | |
| | | UAGGUCCCAU | |
| | | GUUCAAUAGA | |
| | | AGUA/ | |
| | | 3′Biotin/ | |
| UG | 68 | 5′GACGCUUC | 168 |
| | | UGGUACAUUG | |
| | | UAGGUCCCAU | |
| | | CUGCAAUAGA | |
| | | AGUA/ | |
| | | 3′Biotin/ | |
| UC | 76 | 5′GACGCUUC | 169 |
| | | UGGUACAUUG | |
| | | UAGGUCCCAU | |
| | | CUCCAAUAGA | |
| | | AGUA/ | |
| | | 3′Biotin/ | |
| UA | 84 | 5′GACGCUUC | 170 |
| | | UGGUACAUUG | |
| | | UAGGUCCCAU | |
| | | CUACAAUAGA | |
| | | AGUA/ | |
| | | 3′Biotin/ | |
| AU | 66 | 5′GACGCUUC | |
| | | UGGUACAUUG | |
| | | UAGGUCCCAU | |
| | | CAUCAAUAGA | |
| | | AGUA/ | |
| | | 3′Biotin/ | |
| AG | 74 | 5′GACGCUUC | 172 |
| | | UGGUACAUUG | |
| | | UAGGUCCCAU | |
| | | CAGCAAUAGA | |
| | | AGUA/ | |
| | | 3′Biotin/ | |
| AC | 82 | 5′GACGCUUC | 173 |
| | | UGGUACAUUG | |
| | | UAGGUCCCAU | |
| | | CACCAAUAGA | |
| | | AGUA/ | |
| | | 3′Biotin/ | |
| AA | 90 | 5′GACGCUUC | 174 |
| | | UGGUACAUUG | |
| | | UAGGUCCCAU | |
| | | CAACAAUAGA | |
| | | AGUA/ | |
| | | 3′Biotin/ | |
| GU | 62 | 5′GACGCUUC | 175 |
| | | UGGUACAUUG | |
| | | UAGGUCCCAU | |
| | | CGUCAAUAGA | |
| | | AGUA/ | |
| | | 3′Biotin/ | |
| GG | 70 | 5′GACGCUUC | 176 |
| | | UGGUACAUUG | |
| | | UAGGUCCCAU | |
| | | CGGCAAUAGA | |
| | | AGUA/ | |
| | | 3′Biotin/ | |
| GC | 78 | 5′GACGCUUC | 177 |
| | | UGGUACAUUG | |
| | | UAGGUCCCAU | |
| | | CGCCAAUAGA | |
| | | AGUA/ | |
| | | 3′Biotin/ | |
| GA | 86 | 5′GACGCUUC | 176 |
| | | UGGUACAUUG | |
| | | UAGGUCCCAU | |
| | | CGACAAUAGA | |
| | | AGUA/ | |
| | | 3′Biotin/ | |
| CU | 74 | 5′GACGCUUC | 179 |
| | | UGGUACAUUG | |
| | | UAGGUCCCAU | |
| | | CCUCAAUAGA | |
| | | AGUA/ | |
| | | 3′Biotin/ | |
| CG | 72 | 5′GACGCUUC | 180 |
| | | UGGUACAUUG | |
| | | UAGGUCCCAU | |
| | | CCGCAAUAGA | |
| | | AGUA/ | |
| | | 3′Biotin/ | |
| CC | 80 | 5′GACGCUUC | 181 |
| | | UGGUACAUUG | |
| | | UAGGUCCCAU | |
| | | CCCCAAUAGA | |
| | | AGUA/ | |
| | | 3′Biotin/ | |
| CA | 88 | 5′GACGCUUC | 182 |
| | | UGGUACAUUG | |
| | | UAGGUCCCAU | |
| | | CCACAAUAGA | |
| | | AGUA/ | |
| | | 3′Biotin/ | |

### ProRSBL Read-Length Determination

To determine the attainable read length of ProRSBL, interrogation probes consisting of four degenerate bases at positions 1-4, 5-8, or 9-12 were used to interrogate bases either 5′ or 3′ to the sequencing primer and ligation products were quantified using Illumina MiSeq. For forward interrogation (positive position from ligation junction) a 3′ biotinylated RNA template and a 5′ phosphorylated DNA oligo (sequencing primer) complementary to the hybridization site of the RNA template were mixed (5 μM and 10 μM, respectively) in 2×SSC to a total volume of 50 μL. For reverse interrogation, (negative position from ligation junction) a 3′ biotinylated RNA template and a DNA oligo (sequencing primer) complementary to the hybridization site of the RNA template were mixed (5 μM and 10 μM, respectively) in 2×SSC to a total volume of 50 μL. SplintR ligation followed previously described protocol, using 80 μM of total degenerate probes. RNase digestion follows RNA templated ligation protocol. Downstream NGS preparation included PCR amplification with 0.25 μL of each primer, 6.0 μL of ligation product, 25.0 μL of Phusion Pfu high fidelity mastermix (NEB M05315S) and Ultrapure H₂O to 50 μL. Amplified product was cleaned with 2% SizeSelect E-Gel (ThermoFisher G661012), and proceeded with NEB Next Ultra II Library Prep Kit for Illumina (NEB E7645s and E7335s) as per commercial protocol.

| | | SEQ ID |
|---|---|---|
| Description | Sequence | NO: |
| Forward | 5′GACGCUUCUGGUA | 167 |
| sequencing | CAUUGUAGGUCCCAU | |
| RNA | CUUCAAUAGAAGUA/ | |
| | 3′Biotin/ | |
| Reverse | 5′GACGAUUCUGGUU | 183 |
| sequencing | CAUUGUAGGUCCCAU | |
| RNA | CUUCAAUAGAAGUA/ | |
| | 3′Biotin/ | |
| Forwarding | /5′Pbos/AGATGGC | 184 |
| sequencing | ACCTACAATGTACCA | |
| primer | GAAGCGTCTATGACA | |
| | CTCGATCAGGATACA | |
| | CACTACCCGTG3′ | |
| Reverse | 5′TTGGGTCATATCGG | 185 |
| sequencing | TCACTGTTTATGACA | |
| primer | CTCTACTTCTATTGA | |
| | AGATGGGACCTACAA | |
| | TGA3′ | |
| Forward | 5′TTGGGTCATATCG | 186 |
| ProRSBL | GTCACTGTTCGATCG | |
| probe | TCTACTTCTANNNN3 | |
| 1-4 | ′ | |
| Forward | 5′TTGGGTCATATCG | 187 |
| ProRSBL | GTCACTGTTCGATCG | |
| probe | TCTACTNNNNTTGA3′ | |
| 5-8 | | |
| Forward | 5′TTGGGTCATATCG | 188 |
| ProRSBL | GTCACTGTTCGATCG | |
| probe | TCNNNNTCTATTGA3′ | |
| 9-12 | | |
| Reverse | /5′Phos/NNNNGAA | 189 |
| ProRSBL | TCGTCCGATCGTCGA | |
| probe | TCAGGATACACACTA | |
| 1-4 | CCCGTG3′ | |
| Reverse | /5Phos/ACCANNNN | 190 |
| ProRSBL | CGTCCGATCGTCGAT | |
| probe | CAGGATACACACTAC | |
| 5-8 | CCGTG3′ | |
| Reverse | /5′Phos/ACCAGAA | 191 |
| ProRSBL | TNNNNCGATCGTCGA | |
| probe | TCAGGATACACACTA | |
| 9-12 | CCCGG3′ | |

### Endonuclease V Cleavage Kinetics

To determine the kinetics of the cleavage reaction, 200 pmol of a 5′ biotinylated RNA template identical to the sequence of 28s rRNA and 400 pmol of complementary DNA with an inosine (Cleavage substrate 13) were mixed (5 μM and 10 μM, respectively) in 2×SSC to a total volume of 50 μL. The mixture was incubated at 95° C. for 5 min 60° C. for 10 min, and room temperature for 10 min. In the meantime, 100 μL of Dynabeads streptavidin m270 (ThermoFisher, 65306) per each 100 μL volume of oligos were washed in 2×SSC 3-4 times depending on the volume of beads. The hybridized RNA:DNA duplex were added to the washed beads and gently agitated for 15 min at room temperature before being transferred to a magnetic stand for two min and the supernatant removed. The beads were then resuspended in 150 μL of DEPC H₂O to 100 pmol. 60 μL of resuspended beads (20 pmol) were added with 20 μL of NEBuffer 4 10× (ThermoFisher, M0305S) and 60 μL of DEPC H2O. 30 μL was removed for the negative control. 1 uL of EndoV (ThermoFisher, M0305S) was added for a total volume of 170 uL. Cleavage was performed at 37° C. for 60 min with a 20 min heat kill at 65° C. post cleavage. For time courses, reactions were removed from 37° C. at each time point and transferred to a separate 65° C. incubator for a 20 min heat kill. RNase cocktail was created as described above. 10 μL of the cocktail was added directly to cleavage reaction and incubated at 37° C. for 1 hour followed by 10 min of 70° C. The supernatant was removed and diluted 1:2 in Ultrapure H₂O. Product was run on Bioanalyzer AB13730 as in RNA templated ligation protocol.

### Endonuclease V Effect on Total RNA

Human total RNA extracted from Capan-1 (ATCC® HTB-79™) cells was diluted to 50 ng/μL and incubated with 0.1 μL Endonuclease V (10 U/μL) per 10 μL reaction for up to 60 min at 37° C. Samples were heat killed at 65° C. for 20 min following Endonuclease V digestion, and then immediately stored at −80° C. Samples from each timepoint were run on a Nano chip Agilent 2100 Bioanalyzer to inspect integrity via an electronic gel image.

### ProRSBL Following Cleavage

For post cleavage ligation, 500 pmol of a synthetic 5′ biotinylated RNA template and 1000 pmol of complementary DNA with an inosine (Cleavage substrate 18) were mixed (5 μM and 10 μM, respectively) in 2×SSC to a total volume of 50 μL. The mixture was incubated at 95° C. for 5 min, 60° C. for 10 min, and room temperature for 10 min. In the meantime, 100 μL of Dynabeads streptavidin m270 (ThermoFisher, 65306) per each 100 μL volume of oligos were washed in 2×SSC 3-4 times depending on the volume of beads. The hybridized RNA:DNA duplex was added to the washed beads and gently agitated for 15 min at room temperature before being transferred to a magnetic stand for 2 min and the supernatant removed. The beads were then resuspended in 100 μL of DEPC H₂O. 50 μL of resuspended beads were added with 11 μL of NEBuffer 4 10× (ThermoFisher, M0305S) and 39 μL of DEPC H₂O. 10 μL of the mastermix was removed for the negative control. 10 μL of EndoV (ThermoFisher, M0305S) was added for a total volume of 100 μL. Cleavage was performed at 37° C. for 60 min with a 20 min heat kill at 65° C. post cleavage. The reaction was then gently washed in 10 mM Tris twice and resuspended in 100 μL of 10 mM Tris. Beads were then split into two aliquots, 75 μL and 25 μL for positive and negative controls, respectively. A SplintR master mix consisting of 40 μM per base A or T, 1.0 μM of SplintR Ligase (NEB, M0375L) and 1× SplintR buffer to a total volume of 20 μL per reaction. The master mix was added to washed beads, mixed gently, and incubated at 37° C. for 60. Beads were then washed with 10 mM Tris buffer three times. RNase digestion and analysis follow RNA templated ligation protocol.

| | Sequence | SEQ |
|---|---|---|
| | (* = Phosphorothioate | ID |
| Description | bond) | NO: |
| Synthetic | 5′CGUGAGCUGGGUUUAGAC | 192 |
| RNA | CGUCGUGAGACAGGUUAGUU | |
| (from | UUACCCUACUGAUG/ | |
| human | 3′Biotin/ | |
| 28S | | |
| rRNA | | |
| 4490- | | |
| 4540) | | |
| Cleavage | 5′AG/ideoxyl/GT*AAAA | 193 |
| substrate | CTAA*CCTGTCTCACGACGG | |
| 13 | TCTAAACCCAGCFCAC/ | |
| (for | 3′6-FAM/ | |
| cleavage | | |
| kinetics) | | |
| Cleavage | 5′GTAAAAC/ideoxyl/AA | 194 |
| substrate | *CCTGTCTCACGACGGTCTA | |
| 18 | AACCCAGCTCAC/ | |
| (for | 3′6-FAM/ | |
| post | | |
| cleavage | | |
| ligation) | | |
| ligation | 5′GCTAGCTAGCTAGCTAGC | 195 |
| probe | AG/ideoxyl/GT*AAAACT | |
| A | A3′ | |
| ligation | 5′TAGCTAGCTAGCTAGCTA | 196 |
| probe | GCAG/ideoxyl/GT*AAAA | |
| C | CTC3′ | |
| ligation | 5′GCTAGCTAGCTAGCTAGC | 197 |
| probe | TAGCAG/ideoxyl/GT*AA | |
| G | AACTG3′ | |
| ligation | 5′CTGCTAGCTAGCTAGCTA | 198 |
| probe | GCTAGCAG/ideoxyl/GT* | |
| T | AAAACTT3′ | |

### ProRSBL on Synthetic RNA Template for Codon Detection:

A 32 mer RNA polymer (RNA template) flanking different mutation of interest and a 5′ phosphorylated, 3′ biotinylated DNA oligo (sequencing primer) complementary to the hybridization site of the RNA template were mixed (5 μM and 10p M, respectively) in 2×SSC to a total volume of 50 μL. The hybridization, ligation and RNA digestion protocol is the same as mentioned in ProRSBL section. The ligated fragments attached to the beads are diluted 1:10,000 and 1 μL is added to the qPCR reaction mix having 5 μL SYBR green (2×) and 200 μM each of qPCR adapter primer sequence. The qPCR cycles were setup as 95° C. for 5 min, 95° C. for 30 sec, 62° C. for 30 sec, 72° C. for 30 sec, 72° C. for 7 min, 4° C. forever.

### ProRSBL for Codon Detection in Situ:

Adherent immortalized human astrocytes (E6/E7 and hTERT) were cultured on a glass-bottom Mattek dish. The cells are fixed with 2 mL of 10% formalin in PBS for 15 min at 25° C. Cells are washed with 2 mL of PBS three times. Following fixation, 2 mL of 0.25% (vol/vol) Triton X-100 in DEPC-PBS for 10 min. Cells are washed with 2 mL of PBS three times. Cells are treated with 0.1 N HCl in DEPC-treated H₂O for 10 min to improve permeabilization. The sequencing primer (2.5 μM) is added to the cells in presence of 2×SSC containing 10% formamide and SUPERase In (ThermoFisher AM2694) (0.1 U) and incubated overnight at 60° C. in a humidified chamber. The cells are washed with 2 ml of 2×SSC with 10% formamide for three times. The ligation mix is prepared combining 20 μL of 10× SplintR ligase buffer, 5 μL of SplintR Enzyme, 30 μL (10 μM) of each probe interrogating the mutant allele and 10 μL (5 μM) of the wild type probe. DEPC H₂O was added to a total volume of 200 μL.

Ligation was performed at 37° C. for 2 hours followed by washing 3 times with 2 mL 2×SSC containing 10% formamide. Post ligation, the solution was aspirated and 10 μL of DNase-free RNase and 5 μL of RNase H in 1× RNase H buffer was added and incubated for 1 hour at 37° C. The sample was rinsed with 2 mL of nuclease-free 1-O twice to remove traces of phosphate CircLigase II (Lucigen CL9021K) reaction mixture was prepared on ice with 20 μL of 10× CircLigase II buffer, 10 μL (2.5 mM) of 50 mM MnCl₂, 40 μL (0.5 M) of 5M Betaine, 5 μL (1 U μL₋₁) of CircLigase II and Nuclease free H₂O to 200 μL. The master mix was added to the glass bottom dish containing the sample.

Cells were incubated at 60° C. in a humidified chamber for 2 hours. The RCA primer (2.5 M) in 200 μL hybridization buffer containing 2×SSC with 30% formamide was added to glass-bottom dish and incubated at 60° C. for 15 min. The primers were aspirated and washed with 2×SSC with 10% formamide for 10 min at 60° C. followed by wash with 2×SSC for 10 min at 60° C. RCA reaction mixture was prepared on ice with 20 μL of φ29 10× buffer, 373 2 μL (250 μM) of 25 mM dNTPs (Enzymatics N2050L), 2 uL (40 uM) of 4 mM Aminoallyl dUTP (Anaspec AS-83203), 10 μL. (1 U μL₋₁) of φ29 DNA polymerase (Enzymatics P7020-HC-L) and Nuclease free H2e to 200 μL. The master mix was added to the glass-bottom plate. The incubation was performed at 30*C overnight.

To cross-link cDNA molecules containing aminoallyl-dUTP, the RCA reaction mix was aspirated and 20 μL of reconstituted BS(PEG)₉ in 980 μL of PBS was added to the sample and incubated for 1 hour at room temperature. The sample was washed with PBS and incubated with 1 M Tris, pH 8.0 for 30 min. The reaction mix was aspirated and incubated for 10 min at room temperature with 2.5 μM detection probe in 200 μL of 2×SSC preheated at 80° C. The sample was washed three times for 10 min each with gentle shaking.

| | | |
|---|---|---|
| | Sequence | SEQ |
| | (* = Phosphorothioate | ID |
| Description | bond) | NO: |

IDH1 mutant detection

| | | |
|---|---|---|
| Sequencing | /5′Phos/ACCTATGATGAT | 199 |
| primer | AGGTTTTACCCATCCACTCA | |
| | CAATCTCGGGAA3′ | |
| Probe | /5′Phos/CGCTGAAGAAAGC | 200 |
| (ACH) | ATGACH3′ | |
| Probe | /5′Phos/CGCTGAAGAAAGC | 201 |
| (ADG) | ATGADG3′ | |
| Probe | 5′Phos/CGCTGAAGAAAGCA | 202 |
| (BCG) | TGBCG3′ | |
| Probe | /5′InvddT/CGCTGAAGAAA | 203 |
| wildlype | GCATGACG3′ | |
| (ACG) | | |

KRAS mutant detection

| | | |
|---|---|---|
| Synthetic | 5′AACUUGUGGUAGUUGGAGC | 204 |
| template | UGGUGGCGUAGGC3′ | |
| Synthetic | 5′AACUUGUGGUAGUUGGAGC | 205 |
| template | UGAUGGCGUAGGC3′ | |
| (GAU) | | |
| Synthetic | 5′AACUUGUGGUAGUUGGAGC | 206 |
| template | UGUUGGCGUAGGC3′ | |
| (GUU) | | |
| Synthetic | 5′AACUUGUGGUAGUUGGAGC | 207 |
| template | UCGUGGCGUAGGC3′ | |
| (CGU) | | |
| Sequencing | /5′Phos/AGCTCCAACCACC | 208 |
| primer | ACAAGTTTATACTCAGTCAT | |
| | TTCAGGATACACACTACC*C* | |
| | G*PG/3′Biotin/ | |
| Probe | 5′G*G*G*T*CATATCGGTC | 209 |
| (ACD) | ACTGTTTACGCCACD3′ | |
| Probe | 5′G*G*G*T*CATATCGGTC | 210 |
| (ADC) | ACTGTTTACGCCADC3′ | |
| probe | 5′G*G*G*T*CATATCGGTC | 211 |
| (BCC) | ACTGTTTACGCCBCC3′ | |
| probe | 5′TACGCCACC3′ | N/A |
| wildtype | | |
| (ACC) | | |

RCPs were quantified using 8-bit grayscale images of hybridized fluorescent 386 detection oligos that were first filtered by gray morphology erosion operation (gray morphology plugin (2.3.4) in Fiji) using a circle radius of 2 pixels so as to remove speckles and non-RCPs fluorescent signal.

### Discussion of Example 14

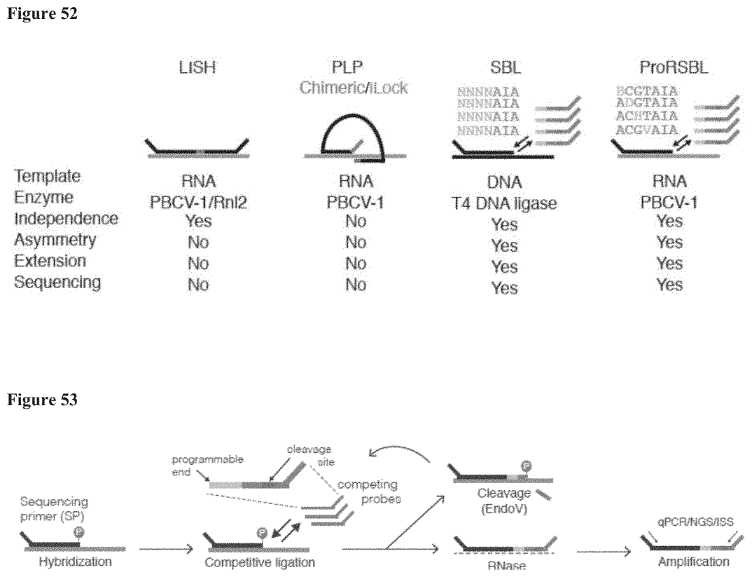

The introduction of T4 RNA ligase 2 (Rnl2) and Chlorella virus DNA ligase (PBCV-1) in place of T4 DNA ligase has increased the efficiency of in situ RNA-templated DNA ligation (RTDL). Current RTDL methods are limited by a number of features: Padlock probe (PLP) approaches rely on connected (dependent) oligo arms that both anneal to the same target with similar kinetics. (FIG. 52). The consequence of oligo dependence is that target sampling involves two stretches of nucleotides and the successful match of one arm is dependent on the success of the other (two body problem). Ligation In Silts Hybridization (LISH) avoids the two-body problem by using independent oligos, one acting as a phosphate donor and the other as an acceptor. However, LIS designs symmetrical donor and acceptor probes that they are equal in length, thereby extending target sampling to both arms (dual-search problem). Moreover, all RTDL methods to date are dead-end reactions, unlike DNA-templated Sequencing By Ligation (SBL) which allows for primer extension and subsequent rounds of nucleic acid discrimination on the same template (FIG. 52). Lastly, current methods are intended to target known sequences (wildtype or specific variants). While our knowledge of pathogenic mutations may warrant limiting searches to what is known, screening for novel variants or indels from genome editing screens requires an agnostic system. Here, we describe Programmable RNA-templated Sequencing By Ligation (ProRSBL), a RTDL framework that overcomes the two-body and dual-search problems. ProRSBL first deploys a long sequencing primer (>30 nt) to hybridize with an RNA target and subsequently introduces shorter competing probes with a melting temperature of ˜37° C. (9-12 nt) in conjunction with PBCV-1 at 37° C. followed by amplification and analysis of the ligated product (FIG. 53). In addition, ProRSBL probes can be cleaved and extended by ligation and can also be programmed to logically enrich for variants without a priori knowledge of their exact sequences (FIG. 53).

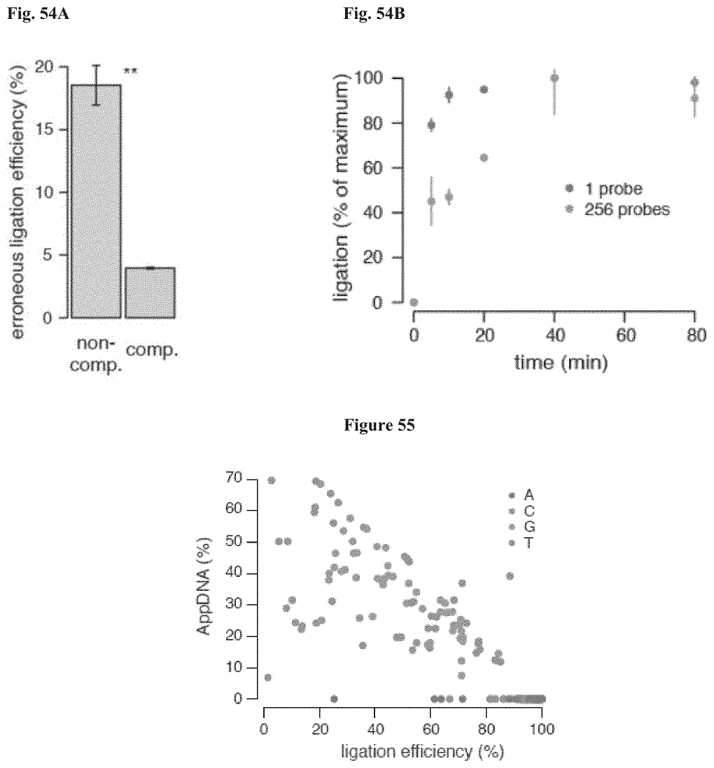

To increase ligation accuracy, we designed ProRSBL so that the sequencing primer anneals more stably to the RNA template compared to the competing probes (Tₘ>60° C., and ˜37° C., respectively). Using this design, we found that probe competition reduced random erroneous ligation from 18.5±1.6% to 4.0±0.1% (P-value=0.0038, Welch Two Sample t-test, t=15.994, df=2.0075) (FIG. 54A), while maintaining similar kinetics as non-competitive ligation with a perfectly matched probe (FIG. 54B). We observed a clear preference for 5′ phosphorylated adenine or thymine (92.9±1.5% and 95.9±0.9%, respectively) and a bias against 5′ cytosine and guanine (47.7±4.1% and 49.5±2.5%, respectively) (FIG. 55 and Table 6 below), consistent with observations made with non-competitive PBCV-1 ligation₄. The reduced ligation efficiency for sequencing primers beginning with C or G (5′ end) correlated with an accumulation of adenylated products (AppDNA), suggesting an inability to complete the last step in RTDL (FIG. 55).

TABLE 6
ProRSBL nucleotide bias (ligation efficiency for
each of the sixteen Donor/Acceptor combinations)

| Donor/Acceptor | Ligation efficiency (%) mean ± stdev | replicates |
|---|---|---|
| 5-A/A-3 | 96.6 ± 2.2 | 14 |
| 5-A/C-3 | 96.5 ± 2.5 | 12 |
| 5-A/G-3 | 92.6 ± 8.7 | 11 |
| 5-A/T-3 | 94.5 ± 3.0 | 12 |
| 5-C/A-3 | 51.1 ± 14.4 | 12 |
| 5-C/C-3 | 68.2 ± 13.0 | 12 |
| 5-C/G-3 | 19.7 ± 12.9 | 12 |
| 5-C/T-3 | 52.1 ± 39.9 | 12 |
| 5-G/A-3 | 33.1 ± 7.5 | 12 |
| 5-G/C-3 | 43.8 ± 17.6 | 12 |
| 5-G/G-3 | 50.6 ± 14.3 | 14 |
| 5-G/T-3 | 70.1 ± 7.5 | 12 |
| 5-T/A-3 | 97.3 ± 3.8 | 12 |
| 5-T/C-3 | 96.7 ± 4.4 | 10 |
| 5-T/G-3 | 96.3 ± 5.5 | 11 |
| 5-T/T-3 | 93.3 ± 10.0 | 11 |

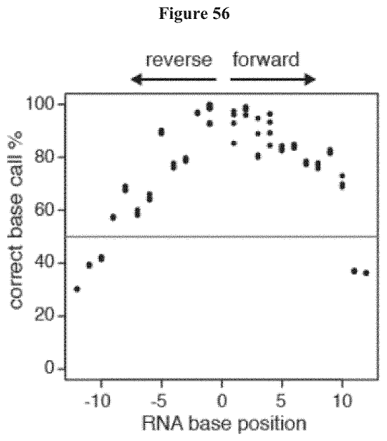

To determine the read length attainable with ProRSBL, we designed probes with degenerate bases interrogating positions 1 to 4, 5 to 8 or 9 to 12 of an RNA template (FIG. 56). By performing forward and reverse ProRSBL followed by next generation sequencing (NGS), we found that the mean base-calling specificity (correct/total base calls) was greater than 90% for the first four bases in either direction and greater than 50% for up to nine bases on both directions of the sequencing primer (FIG. 56). Our results demonstrate that the equilibrium of competing probes increases the specificity of RTDL and that the usable read length of ProRSBL reaches 9 bases upstream or 5 bases downstream the 5′ or 3′ ligation junction site, respectively.

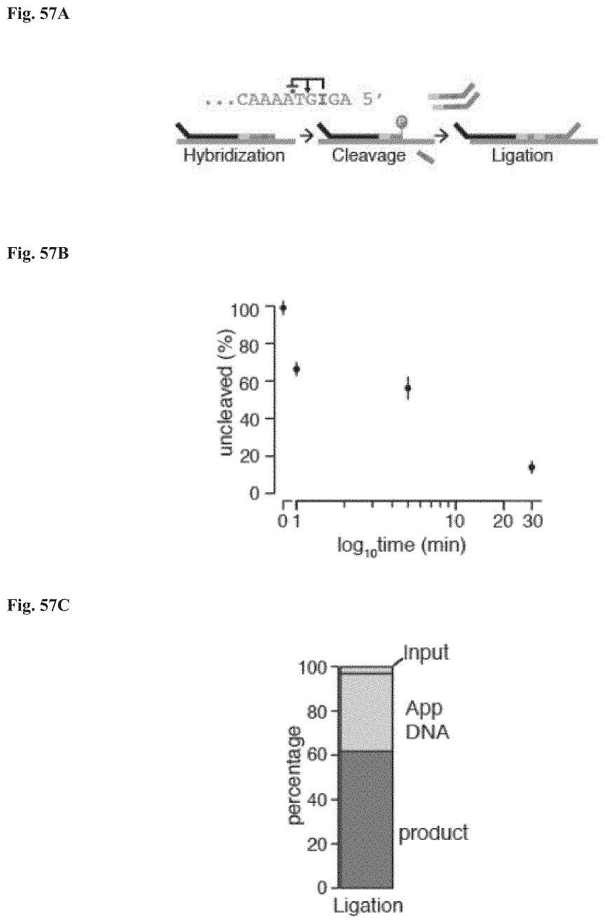

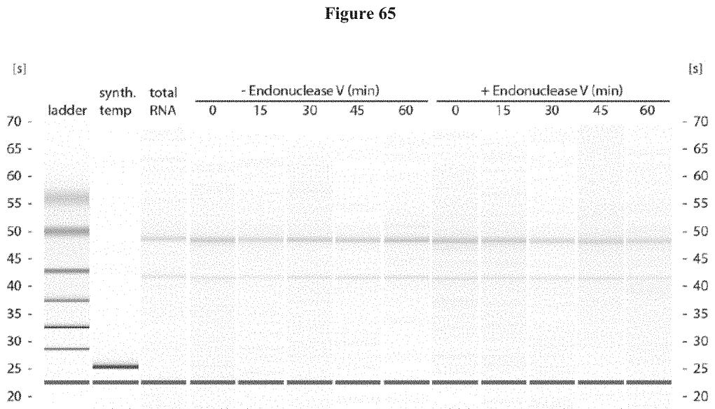

To enable probe cleavage and re-ligation for cyclic ProRSBL, we took advantage of Endonuclease V, which hydrolyzes the second or third phosphodiester bond downstream (3′) of an inosine base The relative rate of hydrolysis is higher at the second phosphodiester bond (95%) and can be increased to 100% if the third bond is substituted with phosphorothioate. Endonuclease V cleavage of DNA-templated SBL ligation products allows multiple ligation cycles to delineate longer sequences. To investigate the feasibility of cyclic RTDL, we first determined the cleavage kinetics of endonuclease V using an inosine-bearing DNA:RNA hybrid and found that cleavage exceeded 75% within 30 minutes (FIG. 57A). Next, we used a cleaved DNA:RNA duplex as substrate for RTDL and found that 61.8±0.8% of the cleaved substrate was correctly ligated after 60 minutes. The remaining substrate either remained as AppDNA (35.1±0.1%) or as cleaved substrate (3.0±0.8%) (FIG. 57B). We did not observe significant degradation of human total RNA in the presence of Endonuclease V for up to 1 hour (FIG. 65).

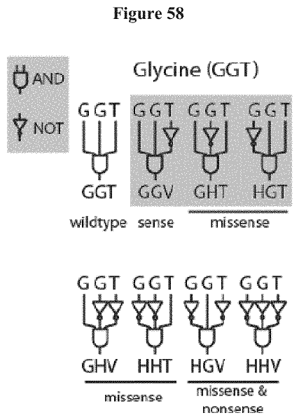

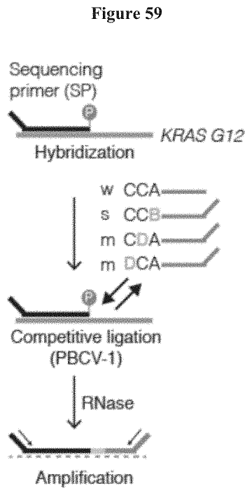

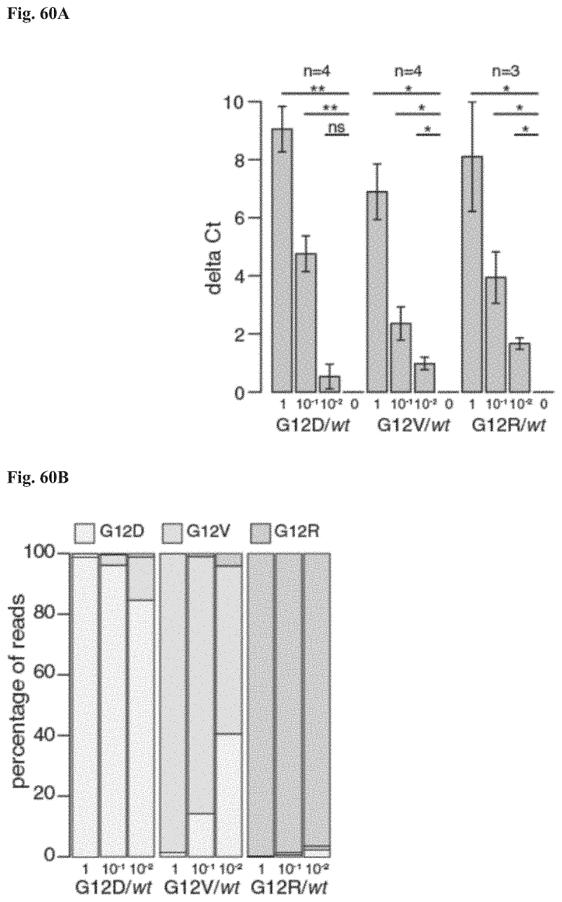

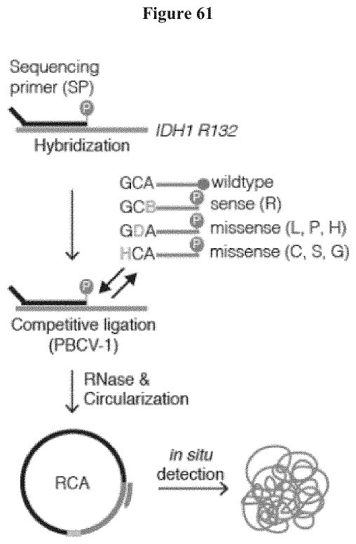

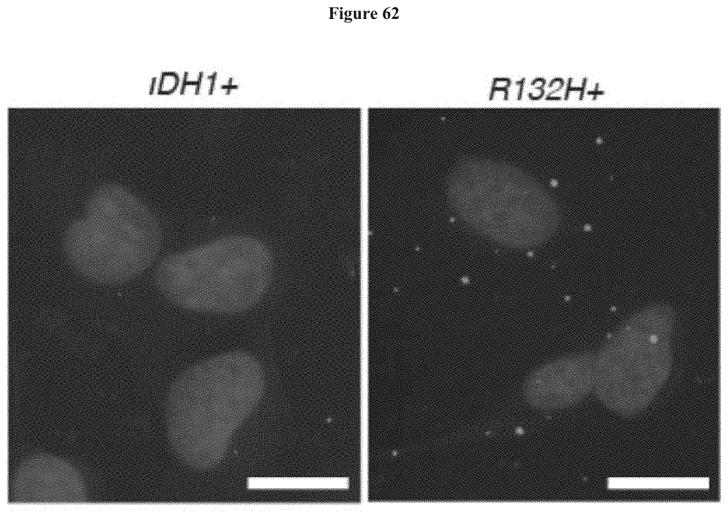

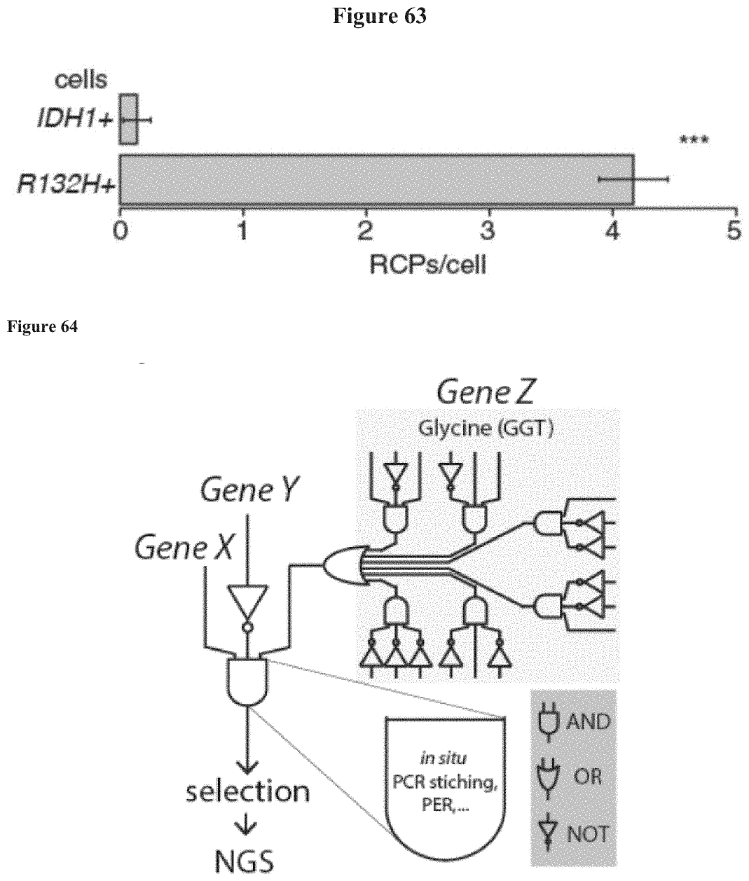

Rare variants (SNPs, alleles, indels etc.) are often masked by the abundance of wildtype sequences. To avoid detecting non-informative sequences, we programmed probes to selectively sequence variants of interest. Our algorithm for probe programming utilizes a NOT logic gate at an interrogated base to exclude ligation products occurring at an undesired sequence (FIG. 58). For example, a probe designed to recognize the wildtype 12th codon of KRAS encoding for Glycine (GGT) should end in 3′-CCA. To amplify ligation products occurring at single-base mutations of the codon in question, we designed three probes with a NOT gate at each of the codon positions (i.e. 3′-DCA, 3′-CDA and 3′-CCB, where D=NOT ‘C’ and B=NOT ‘A’) and added a primer adaptor to the 5′ end of these probes (FIG. 58 and FIG. 59). Conversely, we designed a probe ending in 3′-CCA but without a primer adaptor at the 5′ end, thus excluding ligation products involving the probe recognizing the wildtype codon from subsequent amplifications (FIG. 58 and FIG. 59). ProRSBL on mixed mutant and wildtype synthetic RNA templates followed by qPCR or NGS quantification confirmed that ProRSBL could detect the presence of mutations with a variant allele frequency (VAF) as low as 1% (FIGS. 60A-B). Finally, we modified the arms of the sequencing primer and the programmed probes to allow for post-ligation circularization and rolling circle amplification (RCA) and asked if conditional variant-recognition could be extended in situ. (FIG. 61). Immortalized human astrocytes overexpressing wildtype isocitrate dehydrogenase 1 (IDH1+) or mutant IDH1 (R132H+) were co-cultured with a separating silicon divider. ProRSBL RCA products (RCPs) generated with probes programmed to recognize single-base mutations of IDH1 codon 132 were enriched thirty-fold in R132H+ cells compared to IDH1+ cells (P-value=0.00021 Welch Two Sample t-test, t=13.327, df=3.89) (FIG. 62 and FIG. 63), demonstrated the utility of ProRSBL in situ.

ProRSBL overcomes the two-body and dual-search problems inherent in current RTDL methods. Moreover, the option of cleavage and re-ligation allows for genotyping multiple variants in near proximity on the same RNA. One of the most clinically relevant applications of Pro-RSBL will be in early or disseminated cancer cell detection. However, we foresee the power of ProRSBL to lie in the versatility of programmable probes. Each programmed probe is capable of making a logical statement (AND, OR, NOT), and these statements could be integrated (i.e. via in situ PCR stitching or primer exchange reaction) to assemble a complex statement about the cell in situ. For example, we envision being able to label cells based on conditions such as ‘gene x is expressed, not gene y, but only in the presence of mutation z’ (FIG. 64). Such advanced characterization would allow the enrichment of cells with complex genotypes for deeper analysis using NGS or imaging. Our long-term goal is to distill common algorithms used in genomics and translate them into molecular devices capable of performing basic computations in single cells. We believe that this direction offers a practical way to combine sequencing, computation, and data visualization in every cell across many individuals.

### REFERENCES

Arnold, F. H., et al. (1999). Directed evolution of biocatalysts. Current opinion in chemical biology, 3(1), 54-59.
Bae, T., et al. (2018). Different mutational rates and mechanisms in human cells at pregastrulation and neurogenesis. Science, 359(6375), 550-555.
Credle, J. J., et al. (2017). Multiplexed analysis of fixed tissue RNA using Ligation in situ Hybridization. Nucleic acids research, 45(14), e128-e128.
Dean et al. (2002). Comprehensive human genome amplification using multiple displacement amplification. Proceedings of the National Academy of Sciences, 99(8), 5261-5266.
Enge, M., et al. (2017). Single-cell analysis of human pancreas reveals transcriptional signatures of aging and somatic mutation patterns. Cell, 171(2), 321-330.
Hunziker, J., & Leumann, C. (1995). Nucleic acid analogues: synthesis and properties. Modern Synthetic Methods 1995, 333-417.
Krzywkowski, T., et al. (2017). Fidelity of RNA templated end-joining by chlorella virus DNA ligase and a novel iLock assay with improved direct RNA detection accuracy. Nucleic acids research, 45(18), e161-e161.
Krzywkowski, T., et al. (2019). Chimeric padlock and iLock probes for increased efficiency of targeted RNA detection. RNA, 25(1), 82-89.
Ho, C. K., et al. (1997). Characterization of an ATP-dependent DNA ligase encoded by Chlorella virus PBCV-1. Journal of virology, 71(3), 1931-1937.
Ho, C. K., et al. (2002). Bacteriophage T4 RNA ligase 2 (gp24. 1) exemplifies a family of RNA ligases found in all phylogenetic domains. Proceedings of the National Academy of Sciences, 99(20), 12709-12714.
Landegren, U., et al. (1988). A ligase-mediated gene detection technique. Science, 241(4869), 1077-1080.
Larman, H. B., et al. (2014). Sensitive, multiplex and direct quantification of RNA sequences using a modified RASL assay. Nucleic acids research, 42(14), 9146-9157.
Lee, J. H., et al. (2015). Fluorescent in situ sequencing (FISSEQ) of RNA for gene expression profiling in intact cells and tissues. Nature protocols, 10(3), 442.
Lodato, M. A., et al. (2018). Aging and neurodegeneration are associated with increased mutations in single human neurons. Science, 359(6375), 555-559.
Lohman, G. J., et al. (2013). Efficient DNA ligation in DNA-RNA hybrid helices by Chlorella virus DNA ligase. Nucleic acids research, 42(3), 1831-1844.
Navin, N., et al. (2011). Tumour evolution inferred by single-cell sequencing. Nature, 472(7341), 90.
Shendure, J., et al. (2005). Accurate multiplex polony sequencing of an evolved bacterial genome. Science, 309(5741), 1728-1732.
Turner, N. J. (2009). Directed evolution drives the next generation of biocatalysts. Nature chemical biology, 5(8), 567.
Verma, R., et al. (2012). Computer-aided protein directed evolution: a review of web servers, databases and other computational tools for protein engineering. Computational and structural biotechnology journal, 2(3), e201209008.
Wang, J. S., et al. (2015). Simulation-guided DNA probe design for consistently ultraspecific hybridization. Nature chemistry, 7(7), 545.
Wang, X., et al. (2018). Three-dimensional intact-tissue sequencing of single-cell transcriptional states. Science, 361(6400), eaat5691.
Zhang, D. Y., et al. (2012). Optimizing the specificity of nucleic acid hybridization. Nature chemistry, 4(3), 208.

1. A method for determining the presence or absence of variant ribonucleic acid molecules in a population of ribonucleic acid molecules, wherein the reference sequence of the variant ribonucleic acid molecules is known, the method comprising:
(a) interrogating the population of ribonucleic acid molecules with:
(i) a plurality of primer molecules comprising nucleotides, wherein:
(1) at least 8 consecutive nucleotides, starting at the 5′ or the 3′ end of each primer molecule, are fully complimentary to consecutive nucleotides of the reference sequence, and
(2) the primer molecules have a melting temperature of at least 50° C. when hybridized to their complimentary ribonucleic acid molecules in the population of ribonucleic acid molecules; and
(ii) a plurality of probes comprising L+S nucleotides, wherein:
(1) nucleotides L are complimentary to: (1) the reference sequence that is adjacent to the nucleotides of the reference sequence that each primer molecule is fully complimentary to, or (2) a sequence that differs from (1) at one or more nucleotide bases along the length of L,
(2) nucleotides S are fully complementary to the reference sequence, and
(3) L+S is 8 to 12 and L is at least 1,
so as to saturate the population of ribonucleic acid molecules with the probes and primer molecules such that the probes and primer molecules are adjacent to one another when hybridized to their respective complimentary sequences on the ribonucleic acid molecules, wherein if the 5′ end of the primer molecules are adjacent to the 3′ end of the probes when both are hybridized to their respective complimentary sequences then at least 8 consecutive nucleotides at the 5′ end of the primer molecules are fully complimentary to nucleotides of the reference sequence and the primer molecules have a 5′ phosphorylated A or T, and wherein if the 5′ end of the probes are adjacent to the 3′ end of the primer molecules when both are hybridized to their respective complimentary sequences then at least 8 consecutive nucleotides at the 3′ end of the primer molecules are fully complimentary to nucleotides of the reference sequence and the probes have a 5′ phosphorylated A or T;
(b) ligating the probes to their respective adjacent primer molecules so as to form ligated nucleic acid molecules, wherein the probes are ligated in competition under conditions favoring the ligation of fully hybridized probes over partially hybridized probes, wherein such conditions comprise using a reaction temperature that is about the melting temperature of a probe of length L+S that is fully hybridized;
(c) detecting the presence of ligated nucleic acid molecules that are complementary to a sequence that differs from the reference sequence;
thereby determining the presence or absence of one or more variant ribonucleic acid molecules in the population of ribonucleic acid molecules.

2. The method of claim 1, wherein the primer molecules and probes form the following sequence, read 3′ to 5′, when hybridized to their respective complimentary sequence on a ribonucleic acid molecule in the population of ribonucleic acid molecules, wherein the numbers in brackets represent the number of nucleotides, N represents nucleotides of the primer molecule that are fully complimentary to the reference sequence, P represents additional nucleotides of the primer molecule, and X is any whole number sufficient for the primer molecules to have a melting temperature of at least 50° C. when hybridized to their complimentary ribonucleic acid molecules in the population of ribonucleic acid molecules:
(a) P_{(X)}N₍₈₊₎L₍₁₋₈₎S₍₀₋₁₁₎, wherein the 5′ nucleotide of N is a phosphorylated A or T, or
(b) S₍₀₋₁₁₎L₍₁₋₈₎N₍₈₊₎P_{(X)}, wherein the 5′ nucleotide of L is a phosphorylated A or T
wherein the ligation in step (b) occurs between L and N.

3. The method of claim 1
(a) further comprising a step of removing excess unhybridized or partially hybridized primer molecules and/or probes after step (a);
(b) wherein step (c) comprises sequencing the ligated nucleic acid molecules;
(c) wherein L is 3;
(d) wherein the plurality of probes:
(i) consists of probes complimentary to each respective single base variant along the length of L; or
(ii) consists of probes complimentary to each possible single base variant along the length of L other than non-actionable sequences, synonymous mutations, non-functional polymorphisms, or mutational patterns not observed in the human population;
(e) wherein some or all of the plurality of probes further comprise one or more of:
(i) a signal amplification functional group, preferably horseradish peroxidase, alkaline phosphatase, digoxigenin, or fluorescein isothiocyanate (FITC);
(ii) an amplification sequence, preferably an adapter sequence for circularization and rolling circle amplification (RCA) or a sequence for hybridization of a PCR primer;
(iii) a barcode;
(iv) an inverted dT;
(v) a cleavable terminator, preferably an inosine base; and/or
(vi) a functional group that accepts and transfers energy from an external source for generation of cytotoxic processes;
(f) further comprising a step of degrading un-ligated, excess, and/or off-target probes after step (b);
(g) further comprising a step of amplifying the ligated nucleic acid molecules before step (c).

4-7. (canceled)

8. The method of claim 1, comprising one or more further rounds of interrogation and ligation, wherein the ligated nucleic acid molecules formed in step (b) serve as the primer molecules for the next round of interrogation and ligation with a plurality of probes designed as described in step (a) based on the nucleotides of the reference sequence that are adjacent to the nucleotides of the reference sequence that such ligated nucleic acid molecule are complementary to.

9. The method of claim 8, wherein some or all of the plurality of probes further comprise a cleavable terminator and wherein the cleavable terminator is cleaved to form a cleaved ligated nucleic acid molecules which serve as the primer molecules for the next round of interrogation and ligation with a plurality of probes designed as in step (a) based on the nucleotides reference sequence that are adjacent to the nucleotides of the reference sequence that each cleaved ligated nucleic acid molecule is complementary to.

10. The method of claim 9, wherein Endonuclease V is used to cleave the cleavable terminator of the ligated nucleic acid molecules.

11. The method of claim 1:
(a) wherein (1) probes that are fully complimentary to the reference sequence of the variant ribonucleic acid molecules, and optionally (2) probes that are fully complimentary to non-actionable sequences, synonymous mutations, non-functional polymorphisms, and/or mutational pattern not observed in the human population:
(i) do not further comprise a signal amplification functional group;
(ii) do not further comprise an amplification sequence;
(iii) are tagged with a non-functional control sequence, which control sequence blocks amplification of the ligated nucleic acid molecule; and/or
(iv) comprise an inverted dT to prevent circularization and rolling circle amplification
(b) wherein at least 9 consecutive nucleotides, starting at the 5′ or 3′ end of each primer molecule, are fully complimentary to consecutive nucleotides of the reference sequence;
(c) wherein the primer molecules comprise 20-50 nucleotides, preferably 20-50 nucleotides that are complementary to the reference sequence of the ribonucleic nucleic acid molecules;
(d) wherein each primer molecule further comprises one or more of:
(i) an amplification sequence;
(ii) a signal amplification functional group;
(iii) a blocking group to make the ligated nucleic acid molecules resistant to degradation;
(iv) a fluorescent or colorimetric sequence;
(v) an inverted dT;
(vi) a phosphorothioate nucleotide or phosphorothioate nucleotides;
(vii) a locked nucleic acid or locked nucleic acids;
(viii) a modified base or modified bases;
(ix) a functional group that accepts and transfers energy from an external source for generation of cytotoxic processes.

12-14. (canceled)

15. The method of claim 11, wherein:
(a) each primer molecule comprises a signal amplification functional group wherein the signal amplification functional group is horseradish peroxidase, alkaline phosphatase, digoxigenin, or fluorescein isothiocyanate (FITC);
(b) each primer molecule comprises a blocking group to make the ligated nucleic acid molecules resistant to degradation wherein the blocking group is:
(i) an inverted dT;
(ii) a phosphorothioate nucleotide or phosphorothioate nucleotides;
(iii) inert spacer moiety;
(iv) a locked nucleic acid or locked nucleic acids; or
(v) a modified base or modified bases; and/or
(c) each primer molecule comprises an amplification sequence wherein the amplification sequence:
(i) is an adapter sequence for circularization and rolling circle amplification (RCA); or
(ii) a sequence for hybridization of a PCR primer.

16. (canceled)

17. The method of claim 3, wherein the method comprises a step of degrading un-ligated, excess, and/or off-target probes after step (b), and wherein:
(a) degrading is by an endonuclease, exonuclease, surveyor enzyme, resolvase, or ssDNA-binding protein, preferably wherein:
(i) the exonuclease is Exonuclease I, T7 exonuclease, or Exonuclease III;
(ii) the endonuclease is T7 endonuclease I; and/or
(iii) the exonuclease is used in combination with RNase H and/or an RNase cocktail;
(b) degrading comprises the use of exonucleases that remove bound RNA to degrade partially hybridized probes;
(c) degrading of bound RNA results in the diffusion of the ligated product for in situ applications in fixed cells or tissues;
(d) degrading further comprises hybridization independent degrading;
(e) degradation of ligated nucleic acid molecules is blocked by an inverted dT, phosphorothioate nucleotide, or inert spacer moiety from the primer molecule; and/or
(f) partially hybridized probes of step (b) are in a complex with DNA or RNA molecules or are non-covalently associated with proteins or other cellular material.

18. (canceled)

19. The method of claim 3, wherein the method comprises a step of amplifying the ligated nucleic acid molecules before step (c), and wherein the step of amplifying the ligated nucleic acid molecules comprises:
(a) multiple displacement amplification (MDA);
(b) rolling circle amplification (RCA);
(c) Polymerase chain reaction (PCR) amplification;
(d) first ligating an oligomer assembly to the ligated nucleic acid, wherein the oligomer assembly extends the length of the ligated nucleic acid molecules so as to form an extended ligated nucleic acid molecules, wherein the extended ligated nucleic acid molecules are immobilized; and/or
(e) inhibiting the partially hybridized probe/nucleic acid molecule complexes from being amplified.

20-21. (canceled)

22. The method of claim 19, wherein the step of amplifying the ligated nucleic acid molecules comprises first ligating an oligomer assembly to the ligated nucleic acid, wherein the oligomer assembly extends the length of the ligated nucleic acid molecules so as to form an extended ligated nucleic acid molecules, and wherein:
(a) the oligomer assembly contains multiple copies of the same sequence;
(b) the ligation of the oligomer assembly to the ligated nucleic acid enables degradation of the entire oligomer assembly complex, unless the ligated nucleic acid molecule is exonuclease-resistant, wherein degradation of the oligomer assembly amplifies the detectable signal from ligated nucleic acid molecules that are complementary to a sequence that differs from the reference sequence; and/or
(c) the oligomer assembly is formed by using well, condition, or batch specific monomer sequences that can be grown subsequently using further monomer sequences of alternate sequences for combinatorial labeling of the ligated nucleic acid, preferably wherein the oligomer assembly for combinatorial labeling can be used to multiplex 100 to 1,000,000 single cells or wells, or can be used in high-throughput bulk DNA sequencing.

23. The method of claim 22, wherein the ligation of the oligomer assembly to the ligated nucleic acid enables degradation of the entire oligomer assembly complex, unless the ligated nucleic acid molecule is exonuclease-resistant, wherein degrading of the oligomer assembly complex results in the formation of a single-strand DNA of a known orientation, preferably wherein the single-strand of DNA contains multiple copies of the same sequence corresponding to a sequence of the oligomer assembly, and wherein:
(a) the single strand of DNA can be hybridized and sequenced in situ; or
(b) the single strand of DNA is hybridized to primer molecules linked to magnetic nanoparticles to magnetize the cell for cell purification.

24. (canceled)

25. The method of claim 1, wherein:
(a) 50% of the primer molecules are hybridized within two minutes;
(b) the reaction temperature of step (b) is 37° C.;
(c) the ligating of step (b) is ligation with PBCV ligase, T4 Rnl2, or T4 DNA ligase;
(d) in step (b) partially hybridized probes are ligated to adjacent primer molecules at a rate such that they comprise less than 1 of ligated nucleic acid molecules;
(e) the method can detect the presence of variant ribonucleic acids with a variant allele frequency (VAF) of less than 5%, less than 4%, less than 3%, less than 2%, or about 1%;
(f) the sensitivity of the method to detect variant ribonucleic acid molecules is 75%-90%;
(g) the method is conducted ex vivo;
(h) the method is conducted in vitro;
(i) the method is conducted in situ;
(j) the population of ribonucleic acid molecules:
(i) is in a tissue culture;
(ii) is in a cell culture;
(iii) are bound to a solid support such as a bead;
(iv) are bound to parts of a cell; or
(v) is in a fixed cell or tissue;
(k) the variant ribonucleic acid molecule:
(i) is associated with functional changes, disease, or cancer;
(ii) is used for cell tracing or cell labeling; and/or
(l) the presence or absence of multiple variant ribonucleic acid molecules with different reference sequences is determined by simultaneously performing the method on the population of ribonucleic acid molecules using multiple sets of probes and primer molecules that are each designed as described in step (a) based on the different reference sequences of each of the multiple variant ribonucleic acid molecules.

26-28. (canceled)

29. A composition or kit comprising a primer molecule and at least two probes,
(a) wherein the primer molecule and at least two probes are designed to hybridize to target sequences on a ribonucleic acid molecule such that:
(i) the 5′ end of the primer molecule and the 3′ end of the at least two probes are adjacent when the probe and primer molecule are hybridized to their respective target sequences on the ribonucleic acid molecule; or
(ii) the 5′ end of the at least two probes and the 3′ end of the primer molecule are adjacent when the probe and primer molecule are hybridized to their respective target sequences on the ribonucleic acid molecule;
(b) wherein the primer molecule:
(i) has a melting temperature of at least 50° C. when hybridized to its target sequence;
(ii) if the primer molecule is designed such that the 5′ end of the primer molecule and the 3′ end of the at least two probes are adjacent when the probe and primer molecule are hybridized to their respective target sequences on the ribonucleic acid molecule;
(iii) comprises nucleotides starting at its 5′ end that are fully complimentary to at least 8 consecutive nucleotides of the target sequence and has a 5′ phosphorylated A or T if the primer molecule is designed such that the 5′ end of the primer molecule and the 3′ end of the at least two probes are adjacent when the probe and primer molecule are hybridized to their respective target sequence on the ribonucleic acid molecule; and
(iv) comprises nucleotides starting at its 3′ end that are fully complimentary to at least 8 consecutive nucleotides of the target sequence if the primer molecule is designed such that the 3′ end of the primer molecule and the 5′ end of the at least two probes are adjacent when the probe and primer molecule are hybridized to their respective target sequence on the ribonucleic acid molecule;
(c) wherein the at least two probes:
(i) comprise L+S nucleotides, wherein L+S is 8 to 12, and L is at least 1;
(ii) differ in sequence from one another at only one nucleotide base along the length of L,
(iii) are fully complementary to the target sequence of the probe along the length of S;
(iv) have a 5′ phosphorylated A or T if the at least two probes are designed such that the 5′ end of the at least two probes and the 3′ end of the primer molecule are adjacent when the probes and primer molecule are hybridized to their respective target sequences on the ribonucleic acid molecule.

30. (canceled)

31. The composition or kit of claim 29:
(a) further comprising a ligase, wherein the ligase is preferably PBCV ligase, T4 Rnl2, or T4 DNA ligase;
(b) wherein L is 3;
(c) for use in determining the presence or absence of variant ribonucleic acids in a population of ribonucleic acid molecules;
(d) comprising probes and primers designed as in (a), (b) and (c) to hybridize to multiple different target sequences such that multiple different target sequences can be interrogated in series or preferably simultaneously;
(e) further comprising an endonuclease, exonuclease, surveyor enzyme, resolvase, or ssDNA-binding protein, wherein the exonuclease is preferably Exonuclease I or Exonuclease III, preferably further comprising RNase H and/or an RNase cocktail;
(f) wherein the plurality of probes:
(i) consists of probes complimentary to each respective single base variant along the length of L; or
(ii) consists of probes complimentary to each possible single base variant along the length of L other than non-actionable sequences, synonymous mutations, non-functional polymorphisms, or mutational patterns not observed in the human population;
(g) wherein some or all of the plurality of probes further comprise one or more of:
(i) a signal amplification functional group;
(ii) an amplification sequence;
(iii) a barcode;
(iv) an inverted dT;
(v) a cleavable terminator; and
(vi) a functional group that accepts and transfers energy from an external source for generation of cytotoxic processes;
(h) wherein some or all of the plurality of probes further comprise a cleavable terminator and Endonuclease V is used to cleave the terminator of the ligated nucleic acid molecule;
(i) wherein (1) probes that are fully complimentary to the reference sequence of the variant ribonucleic acid molecules, and preferably (2) probes that are fully complimentary to non-actionable sequences, synonymous mutations, non-functional polymorphisms, and/or mutational pattern not observed in the human population:
(i) do not further comprise a signal amplification functional group;
(ii) do not further comprise an amplification sequence;
(iii) are tagged with a non-functional control sequence, which control sequence blocks amplification of the ligated nucleic acid molecule; and/or
(iv) comprise an inverted dT to prevent circularization and rolling circle amplification;
(j) wherein at least 9 consecutive nucleotides, starting at the 5′ or 3′ end of each primer molecule, are fully complimentary to consecutive nucleotides of the reference sequence,
(k) wherein the primer molecule comprises 20-50 nucleotides that are complementary to the reference sequence of the ribonucleic nucleic acid molecules;
(l) wherein each primer molecule further comprises one or more of:
(i) an amplification sequence;
(ii) a signal amplification functional group;
(iii) a blocking group to make the ligated nucleic acid molecules resistant to degradation;
(iv) a fluorescent or colorimetric sequence;
(v) an inverted dT;
(vi) a phosphorothioate nucleotide or phosphorothioate nucleotides;
(vii) a locked nucleic acid or locked nucleic acids;
(viii) a modified base or modified bases; and
(ix) a functional group that accepts and transfers energy from an external source for generation of cytotoxic processes.

32-37. (canceled)

38. The composition or kit of claim 29, wherein:
(a) some or all of the plurality of probes comprise a signal amplification functional group wherein the signal amplification functional group is horseradish peroxidase, alkaline phosphatase, digoxigenin, or fluorescein isothiocyanate (FITC);
(b) some or all of the plurality of probes comprise an amplification sequence and the amplification sequence is:
(i) an adapter sequence for circularization and rolling circle amplification (RCA); or
(ii) a sequence for hybridization of a PCR primer,
(c) some or all of the plurality of probes comprise a cleavable terminator and the cleavable terminator is an inosine base.

39-43. (canceled)

44. The composition or kit of claim 31, wherein:
(a) each primer molecule comprises a blocking group to make the ligated nucleic acid molecules resistant to degradation wherein the blocking group is:
(i) an inverted dT;
(ii) a phosphorothioate nucleotide or phosphorothioate nucleotides;
(iii) inert spacer moiety;
(iv) a locked nucleic acid or locked nucleic acids; or
(v) a modified base or modified bases;
(b) each primer molecule comprises an amplification sequence wherein the amplification sequence:
(i) is an adapter sequence for circularization and rolling circle amplification (RCA); or
(ii) a sequence for hybridization of a PCR primer.

45. A composition comprising complexes of primer molecules, probes and ribonucleic acid molecules,
(a) wherein the complexes comprise primer molecules and probes that are hybridized to target sequences on the ribonucleic acid molecules such that:
(i) the 5′ end of the primer molecules and the 3′ end of the probes are adjacent when hybridized their respective target sequences on the ribonucleic acid molecules; or
(ii) the 5′ end of the at least two probes and the 3′ end of the primer molecule are adjacent when hybridized their respective target sequences on the ribonucleic acid molecules;
(b) wherein the primer molecules:
(i) have a melting temperature of at least 50° C. when hybridized to their target sequence;
(ii) comprise nucleotides that are fully complimentary to at least 8 consecutive nucleotides of the target sequence at the 5′ end of the primer molecule and have a 5′ phosphorylated A or T if the primer molecules are hybridized to their target sequence on the ribonucleic acid molecules such that the 5′ end of the primer molecules and the 3′ end of the probes are adjacent; and
(iii) comprise nucleotides that are fully complimentary to at least 8 consecutive nucleotides of the target sequence at the 3′ end of the primer molecule if the primer molecules are hybridized to their target sequence on the ribonucleic acid molecules such that the 3′ end of the primer molecules and the 5′ end of the probes are adjacent; and
(c) wherein the composition comprises at least two probes, wherein such probes:
(i) comprise L+S nucleotides, wherein L+S is 8 to 12, and L is at least 1;
(ii) differ in sequence from one another at only one nucleotide base along the length of L,
(iii) are fully complementary to the target sequence of the at least two probes along the length of S;
(iv) have a 5′ phosphorylated A or T if the probes are hybridized to their target sequence on the ribonucleic acid molecules such that the 3′ end of the primer molecules and the 5′ end of the probes are adjacent.

46. The composition of claim 45, wherein the complexes further comprise a ligase.

47. A method of treating a disease or condition associated with the presence of variant ribonucleic acid molecules in a subject, the method comprising:
(a) using the method of claim 1 to determine the presence or absence of a variant ribonucleic acid molecule in the subject; and
(b) treating the subject based on the presence or absence of the variant ribonucleic acid molecule.
